(19) United States
(12) Patent Application Publication
(10) Pub. No.: US 2023/0303654 A1
(43) Pub. Date: Sep. 28, 2023

(54) COMPOSITIONS AND USES OF SARS-COV-2 TARGETED CHIMERIC ANTIGEN RECEPTOR MODIFIED NK CELLS

(71) Applicant: City of Hope, Duarte, CA (US)

(72) Inventors: Jianhua Yu, Duarte, CA (US); Michael A. Caligiuri, Pasadena, CA (US)

(21) Appl. No.: 18/020,858

(22) PCT Filed: Aug. 11, 2021
(86) PCT No.: PCT/US2021/045603

§ 371 Date: Feb. 10, 2023

Related U.S. Application Data
(60) Provisional application No. 63/064,381, filed on Aug. 11, 2020.
(60) Provisional application No. 63/064,388, filed on Aug. 11, 2020.
(60) Provisional application No. 63/064,389, filed on Aug. 11, 2020.

(51) Int. Cl. C07K 14/725; C07K 14/705; C07K 14/54; C12N 15/63; A61K 35/17; C12N 5/0783; A61P 31/14

(52) U.S. Cl. CPC C07K 14/7051; C07K 14/70521; C07K 14/70517; C07K 14/5443; C12N 15/63; A61K 35/17; C12N 5/0646; A61P 31/14

(57) ABSTRACT

Chimeric antigen receptors (CAR) targeted to SARS-CoV-2 Spike protein and NK cells expressing such CAR are described. The CAR are targeted to SARS-CoV-2 Spike protein via an scFv or a variant human ACE2 protein. In some cases, the NK cells also express human IL-15 or a soluble fragment thereof.

### CLAIM OF PRIORITY

This application claims the benefit of U.S. Provisional Application Ser. No. 63/064,381, filed on Aug. 11, 2020, 63/064,388, filed on Aug. 11, 2020, and 63/064,389, filed on Aug. 11, 2020. The entire contents of the foregoing are incorporated herein by reference.

### TECHNICAL FIELD

This disclosure concerns SARS-COV-2 targeted chimeric antigen receptor (CAR)-engineered NK cells, methods of formulating, and methods of use.

### BACKGROUND

In both SARS-CoV-1 and SARS-CoV-2 infections, the coronavirus spike (S) glycoprotein promotes SARS-CoV-2 entry into host cells via the host receptor angiotensin (Ang) converting enzyme 2 (ACE2).

### SUMMARY

Described herein are methods for making and using CAR targeted to SARS-CoV-2 Spike protein and NK cells expressing such CAR. The CAR are targeted to SARS-CoV-2 Spike protein via an scFv or a variant human ACE2 protein. In some cases, the NK cells also express human IL-15 or a soluble fragment thereof.

Described herein are nucleic acid molecules comprising a nucleotide sequence encoding a chimeric antigen receptor (CAR), wherein the chimeric antigen receptor comprises: an scFv targeting SAR-CoV2 Spike Protein, a spacer, a transmembrane domain, a co-stimulatory domain, and a CD3ζ signaling domain. In various embodiments: the scFv comprises the amino acid sequence of any of SEQ ID NOs:1, 41-45 or variant thereof having 1-5 amino acid modifications; and the scFv comprises the heavy and light chain CDRs of any amino acid sequence of SEQ ID NOs:1, 41-45.

Also described herein are nucleic acid molecules comprising a nucleotide sequence encoding a chimeric antigen receptor (CAR), wherein the chimeric antigen receptor comprises: a variant of human ACE2 and its mutated counterparts, a spacer, a transmembrane domain, a co-stimulatory domain, and a CD3ζ signaling domain. In various embodiments: the variant human ACE2 comprises the amino acid sequence of any of SEQ ID NOs:29, 30, 31, 32, 33, 38, 39, and 40, or variant thereof having 1-5 amino acid modifications.

In various embodiments: the transmembrane domain is selected from: a CD4 transmembrane domain or variant thereof having 1-5 amino acid modifications, a CD8 transmembrane domain or variant thereof having 1-5 amino acid modifications, a CD28 transmembrane domain or a variant thereof having 1-5 amino acid modifications; the transmembrane domain is a CD28 transmembrane domain; the costimulatory domain is a CD28, 4-1BB, or CD28gg; the costimulatory domain comprises the amino acid sequence of SEQ ID NO:22, 23, or 24 or a variant thereof having 1-5 amino acid modifications; the CD3ζ signaling domain comprises the amino acid sequence of SEQ ID NO:21; a linker of 3 to 15 amino acids is located between the costimulatory domain and the CD3ζ signaling domain or variant thereof; the spacer comprises any one of SEQ ID NOs:2-12 or a variant thereof having 1-5 amino acid modifications; the transmembrane domain is selected from: a CD4 transmembrane domain or variant thereof having 1-5 amino acid modifications, a CD8 transmembrane domain or variant thereof having 1-5 amino acid modifications, a CD28 transmembrane domain or a variant thereof having 1-5 amino acid modifications; the transmembrane domain is a CD28 transmembrane domain.

Also described herein is an expression vector comprising the nucleic acid molecule described herein. In various embodiments: the vector further comprises a sequence encoding human IL-15 or a soluble fragment thereof; the vector is a viral vector (e.g., a lentiviral vector). Also described herein is a population of human NK cells transduced by a vector comprising a nucleic acid molecule described herein or comprising a nucleic acid molecule described herein.

The CAR can also be expressed in various T cell populations, dendritic cells or macrophages.

Also described is method of treating a patient infected with SARS-CoV-2 comprising administering a composition comprising the population of human NK cells expressing a Spike CAR described herein wherein the cells are autologous or allogenic to the patient. Also described is a method of reducing or eliminating SARS-CoV-2 in a subject comprising administering a population of autologous or allogeneic NK cells transduced by a vector comprising a nucleic acid molecule described herein. NK cells can be generated from peripheral blood, umbilical cord blood, induced pluripotent stem cells iPSC, hematopoietic stem cells, etc.

### Spike Protein-Targeted CAR

The Spike protein targeted CAR (also called “Spike CAR”) described herein include a Spike protein targeted scFv or a variant of human ACE2, e.g., a deletion mutant or a point mutant.

### ACE2 Variants

Suitable ACE2 variants for incorporation into a Spike CAR include:

| hACE2 WT-long extracellular domain | |
|---|---|
| (SEQ ID NO: 29) | |
| MSSSSWLLLSLVAVTAAQSTIEEQAKTFLDKFNHEAEDLFYQSSLASWNYNTNITEENVQ | 60 |
| NMNNAGDKWSAFLKEQSTLAQMYPLQEIQNLTVKLQLQALQQNGSSVLSEDKSKRLNTIL | 120 |
| NTMSTIYSTGKVCNPDNPQECLLLEPGLNEIMANSLDYNERLWAWESWRSEVGKQLRPLY | 180 |
| EEYVVLKNEMARANHYEDYGDYWRGDYEVNGVDGYDYSRGQLIEDVEHTFEEIKPLYEHL | 240 |
| HAYVRAKLMNAYPSYISPIGCLPAHLLGDMWGRFWTNLYSLTVPFGQKPNIDVTDAMVDQ | 300 |
| AWDAQRIFKEAEKFFVSVGLPNMTQGFWENSMLTDPGNVQKAVCHPTAWDLGKGDFRILM | 360 |
| CTKVTMDDFLTAHHEMGHIQYDMAYAAQPFLLRNGANEGFHEAVGEIMSLSAATPKHLKS | 420 |
| IGLLSPDFQEDNETEINFLLKQALTIVGTLPFTYMLEKWRWMVFKGEIPKDQWMKKWWEM | 480 |
| KREIVGVVEPVPHDETYCDPASLFHVSNDYSFIRYYTRTLYQFQFQEALCQAAKHEGPLH | 540 |
| KCDISNSTEAGQKLFNMLRLGKSEPWTLALENVVGAKNMNVRPLLNYFEPLFTWLKDQNK | 600 |
| NSFVGWSTDWSPYADQSIKVRISLKSALGDKAYEWNDNEMYLFRSSVAYAMRQYFLKVKN | 660 |
| QMILFGEEDVRVANLKPRISFNFFVTAPKNVSDIIPRTEVEKAIRMSRSRINDAFRLNDN | 720 |
| SLEFLGIQPTLGPPNQPPPVS | 740 |
| hACE2 WT-short extracellular domain | |
| (SEQ ID NO: 30) | |
| MSSSSWLLLSLVAVTAAQSTIEEQAKTFLDKFNHEAEDLFYQSSLASWNYNTNITEENVQ | 60 |
| NMNNAGDKWSAFLKEQSTLAQMYPLQEIQNLTVKLQLQALQQNGSSVLSEDKSKRLNTIL | 120 |
| NTMSTIYSTGKVCNPDNPQECLLLEPGLNEIMANSLDYNERLWAWESWRSEVGKQLRPLY | 180 |
| EEYVVLKNEMARANHYEDYGDYWRGDYEVNGVDGYDYSRGQLIEDVEHTFEEIKPLYEHL | 240 |
| HAYVRAKLMNAYPSYISPIGCLPAHLLGDMWGRFWTNLYSLTVPFGQKPNIDVTDAMVDQ | 300 |
| AWDAQRIFKEAEKFFVSVGLPNMTQGFWENSMLTDPGNVQKAVCHPTAWDLGKGDFRILM | 360 |
| CTKVTMDDFLTAHHEMGHIQYDMAYAAQPFLLRNGANEGFHEAVGEIMSLSAATPKHLKS | 420 |
| IGLLSPDFQEDNETEINFLLKQALTIVGTLPFTYMLEKWRWMVFKGEIPKDQWMKKWWEM | 480 |
| KREIVGVVEPVPHDETYCDPASLFHVSNDYSFIRYYTRTLYQFQFQEALCQAAKHEGPLH | 540 |
| KCDISNSTEAGQKLFNMLRLGKSEPWTLALENVVGAKNMNVRPLLNYFEPLFTWLKDQNK | 600 |
| NSFVGWSTDWSPYAD | 615 |
| hACE2 R273S point mutant long | |
| (SEQ ID NO: 31) | |
| MSSSSWLLLSLVAVTAAQSTIEEQAKTFLDKFNHEAEDLFYQSSLASWNYNTNITEENVQ | 60 |
| NMNNAGDKWSAFLKEQSTLAQMYPLQEIQNLTVKLQLQALQQNGSSVLSEDKSKRLNTIL | 120 |
| NTMSTIYSTGKVCNPDNPQECLLLEPGLNEIMANSLDYNERLWAWESWRSEVGKQLRPLY | 180 |
| EEYVVLKNEMARANHYEDYGDYWRGDYEVNGVDGYDYSRGQLIEDVEHTFEEIKPLYEHL | 240 |
| HAYVRAKLMNAYPSYISPIGCLPAHLLGDMWGSFWTNLYSLTVPFGQKPNIDVTDAMVDQ | 300 |
| AWDAQRIFKEAEKFFVSVGLPNMTQGFWENSMLTDPGNVQKAVCHPTAWDLGKGDFRILM | 360 |
| CTKVTMDDFLTAHHEMGHIQYDMAYAAQPFLLRNGANEGFHEAVGEIMSLSAATPKHLKS | 420 |
| IGLLSPDFQEDNETEINFLLKQALTIVGTLPFTYMLEKWRWMVFKGEIPKDQWMKKWWEM | 480 |
| KREIVGVVEPVPHDETYCDPASLFHVSNDYSFIRYYTRTLYQFQFQEALCQAAKHEGPLH | 540 |
| KCDISNSTEAGQKLFNMLRLGKSEPWTLALENVVGAKNMNVRPLLNYFEPLFTWLKDQNK | 600 |
| NSFVGWSTDWSPYADQSIKVRISLKSALGDKAYEWNDNEMYLFRSSVAYAMRQYFLKVKN | 660 |
| QMILFGEEDVRVANLKPRISFNFFVTAPKNVSDIIPRTEVEKAIRMSRSRINDAFRLNDN | 720 |
| SLEFLGIQPTLGPPNQPPVS | 740 |
| hACE2 H345F point mutant long | |
| (SEQ ID NO: 32) | |
| MSSSSWLLLSLVAVTAAQSTIEEQAKTFLDKFNHEAEDLFYQSSLASWNYNTNITEENVQ | 60 |
| NMNNAGDKWSAFLKEQSTLAQMYPLQEIQNLTVKLQLQALQQNGSSVLSEDKSKRLNTIL | 120 |
| NTMSTIYSTGKVCNPDNPQECLLLEPGLNEIMANSLDYNERLWAWESWRSEVGKQLRPLY | 180 |
| EEYVVLKNEMARANHYEDYGDYWRGDYEVNGVDGYDYSRGQLIEDVEHTFEEIKPLYEHL | 240 |
| HAYVRAKLMNAYPSYISPIGCLPAHLLGDMWGRFWTNLYSLTVPFGQKPNIDVTDAMVDQ | 300 |
| AWDAQRIFKEAEKFFVSVGLPNMTQGFWENSMLTDPGNVQKAVCFPTAWDLGKGDFRILM | 360 |
| CTKVTMDDFLTAHHEMGHIQYDMAYAAQPFLLRNGANEGFHEAVGEIMSLSAATPKHLKS | 420 |
| IGLLSPDFQEDNETEINFLLKQALTIVGTLPFTYMLEKWRWMVFKGEIPKDQWMKKWWEM | 480 |
| KREIVGVVEPVPHDETYCDPASLFHVSNDYSFIRYYTRTLYQFQFQEALCQAAKHEGPLH | 540 |
| KCDISNSTEAGQKLFNMLRLGKSEPWTLALENVVGAKNMNVRPLLNYFEPLFTWLKDQNK | 600 |
| NSFVGWSTDWSPYADQSIKVRISLKSALGDKAYEWNDNEMYLFRSSVAYAMRQYFLKVKN | 660 |
| QMILFGEEDVRVANLKPRISFNFFVTAPKNVSDIIPRTEVEKAIRMSRSRINDAFRLNDN | 720 |
| SLEFLGIQPTLGPPNQPPVS | 740 |
| hACE2 H345S point mutant long | |
| (SEQ ID NO: 33) | |
| MSSSSWLLLSLVAVTAAQSTIEEQAKTFLDKFNHEAEDLFYQSSLASWNYNTNITEENVQ | 60 |
| NMNNAGDKWSAFLKEQSTLAQMYPLQEIQNLTVKLQLQALQQNGSSVLSEDKSKRLNTIL | 120 |
| NTMSTIYSTGKVCNPDNPQECLLLEPGLNEIMANSLDYNERLWAWESWRSEVGKQLRPLY | 180 |
| EEYVVLKNEMARANHYEDYGDYWRGDYEVNGVDGYDYSRGQLIEDVEHTFEEIKPLYEHL | 240 |
| HAYVRAKLMNAYPSYISPIGCLPAHLLGDMWGRFWTNLYSLTVPFGQKPNIDVTDAMVDQ | 300 |
| AWDAQRIFKEAEKFFVSVGLPNMTQGFWENSMLTDPGNVQKAVCSPTAWDLGKGDFRILM | 360 |
| CTKVTMDDFLTAHHEMGHIQYDMAYAAQPFLLRNGANEGFHEAVGEIMSLSAATPKHLKS | 420 |
| IGLLSPDFQEDNETEINFLLKQALTIVGTLPFTYMLEKWRWMVFKGEIPKDQWMKKWWEM | 480 |
| KREIVGVVEPVPHDETYCDPASLFHVSNDYSFIRYYTRTLYQFQFQEALCQAAKHEGPLH | 540 |
| KCDISNSTEAGQKLFNMLRLGKSEPWTLALENVVGAKNMNVRPLLNYFEPLFTWLKDQNK | 600 |
| NSFVGWSTDWSPYADQSIKVRISLKSALGDKAYEWNDNEMYLFRSSVAYAMRQYFLKVKN | 660 |
| QMILFGEEDVRVANLKPRISFNFFVTAPKNVSDIIPRTEVEKAIRMSRSRINDAFRLNDN | 720 |
| SLEFLGIQPTLGPPNQPPVS | 740 |
| hACE2 R273S point mutant short | |
| (SEQ ID NO: 38) | |
| MSSSSWLLLSLVAVTAAQSTIEEQAKTFLDKFNHEAEDLFYQSSLASWNYNTNITEENVQ | 60 |
| NMNNAGDKWSAFLKEQSTLAQMYPLQEIQNLTVKLQLQALQQNGSSVLSEDKSKRLNTIL | 120 |
| NTMSTIYSTGKVCNPDNPQECLLLEPGLNEIMANSLDYNERLWAWESWRSEVGKQLRPLY | 180 |
| EEYVVLKNEMARANHYEDYGDYWRGDYEVNGVDGYDYSRGQLIEDVEHTFEEIKPLYEHL | 240 |
| HAYVRAKLMNAYPSYISPIGCLPAHLLGDMWGSFWTNLYSLTVPFGQKPNIDVTDAMVDQ | 300 |
| AWDAQRIFKEAEKFFVSVGLPNMTQGFWENSMLTDPGNVQKAVCHPTAWDLGKGDFRILM | 360 |
| CTKVTMDDFLTAHHEMGHIQYDMAYAAQPFLLRNGANEGFHEAVGEIMSLSAATPKHLKS | 420 |
| IGLLSPDFQEDNETEINFLLKQALTIVGTLPFTYMLEKWRWMVFKGEIPKDQWMKKWWEM | 480 |
| KREIVGVVEPVPHDETYCDPASLFHVSNDYSFIRYYTRTLYQFQFQEALCQAAKHEGPLH | 540 |
| KCDISNSTEAGQKLFNMLRLGKSEPWTLALENVVGAKNMNVRPLLNYFEPLFTWLKDQNK | 600 |
| NSFVGWSTDWSPYAD | 660 |
| hACE2 H345F point mutant short | |
| (SEQ ID NO: 39) | |
| MSSSSWLLLSLVAVTAAQSTIEEQAKTFLDKFNHEAEDLFYQSSLASWNYNTNITEENVQ | 60 |
| NMNNAGDKWSAFLKEQSTLAQMYPLQEIQNLTVKLQLQALQQNGSSVLSEDKSKRLNTIL | 120 |
| NTMSTIYSTGKVCNPDNPQECLLLEPGLNEIMANSLDYNERLWAWESWRSEVGKQLRPLY | 180 |
| EEYVVLKNEMARANHYEDYGDYWRGDYEVNGVDGYDYSRGQLIEDVEHTFEEIKPLYEHL | 240 |
| HAYVRAKLMNAYPSYISPIGCLPAHLLGDMWGRFWTNLYSLTVPFGQKPNIDVTDAMVDQ | 300 |
| AWDAQRIFKEAEKFFVSVGLPNMTQGFWENSMLTDPGNVQKAVCFPTAWDLGKGDFRILM | 360 |
| CTKVTMDDFLTAHHEMGHIQYDMAYAAQPFLLRNGANEGFHEAVGEIMSLSAATPKHLKS | 420 |
| IGLLSPDFQEDNETEINFLLKQALTIVGTLPFTYMLEKWRWMVFKGEIPKDQWMKKWWEM | 480 |
| KREIVGVVEPVPHDETYCDPASLFHVSNDYSFIRYYTRTLYQFQFQEALCQAAKHEGPLH | 540 |
| KCDISNSTEAGQKLFNMLRLGKSEPWTLALENVVGAKNMNVRPLLNYFEPLFTWLKDQNK | 600 |
| NSFVGWSTDWSPYAD | |
| hACE2 H345S point mutant long | |
| (SEQ ID NO: 40) | |
| MSSSSWLLLSLVAVTAAQSTIEEQAKTFLDKFNHEAEDLFYQSSLASWNYNTNITEENVQ | 60 |
| NMNNAGDKWSAFLKEQSTLAQMYPLQEIQNLTVKLQLQALQQNGSSVLSEDKSKRLNTIL | 120 |
| NTMSTIYSTGKVCNPDNPQECLLLEPGLNEIMANSLDYNERLWAWESWRSEVGKQLRPLY | 180 |
| EEYVVLKNEMARANHYEDYGDYWRGDYEVNGVDGYDYSRGQLIEDVEHTFEEIKPLYEHL | 240 |
| HAYVRAKLMNAYPSYISPIGCLPAHLLGDMWGRFWTNLYSLTVPFGQKPNIDVTDAMVDQ | 300 |
| AWDAQRIFKEAEKFFVSVGLPNMTQGFWENSMLTDPGNVQKAVCSPTAWDLGKGDFRILM | 360 |
| CTKVTMDDFLTAHHEMGHIQYDMAYAAQPFLLRNGANEGFHEAVGEIMSLSAATPKHLKS | 420 |
| IGLLSPDFQEDNETEINFLLKQALTIVGTLPFTYMLEKWRWMVFKGEIPKDQWMKKWWEM | 480 |
| KREIVGVVEPVPHDETYCDPASLFHVSNDYSFIRYYTRTLYQFQFQEALCQAAKHEGPLH | 540 |
| KCDISNSTEAGQKLFNMLRLGKSEPWTLALENVVGAKNMNVRPLLNYFEPLFTWLKDQNK | 600 |
| NSFVGWSTDWSPYAD | |
| ScFv Targeted to Spike Protein | |
| Suitable scFv targeted to Spike protein for incorporation into Spike | |
| CAR include: | |
| Anti-spike RBD Ab38 | |
| (SEQ ID NO: 1) | |
| ELQMTQSPSSLSASVGDRVTITCRASQSISSYLNWYQQKPGKAPKLLIYAASSLQSGVPS | 60 |
| L-CDR1 L-CDR2 | |
| RFSGSGSGTDFTLTISSLQPEDFATYYCQQANSFPLTFGGGTKVEIKGGSSRSSQVQLVQ | 120 |
| L-CDR3 | |
| SGAEVKKPGSSVKVSCKASGGTLSENGFSWVRQAPGQGLEWMGRIVPMFDISNYAERFQD | 180 |
| H-CDR1 H-CDR12 | |
| RVTITADGSTNTAYMELRSLRLEDTAVYYCARDAVATSPGVYQHYGMDVWGQGTTVTVSS | 240 |
| H-CDR3 | |
| ASTKGPSV | |
| Anti-spike RBD Ab24 | |
| (SEQ ID NO: 41) | |
| ELVMTQSPSSLSASVGDRVTITCRASQSISSYLNWYQQKPGKAPKLLIYAASSLQSGVPS | 60 |
| L-CDR1 L-CDR2 | |
| RFSGSGSGTDFTLTISSLQPEDFATYYCQQSYSTPLTFGPGTKVDIKGGSSRSSEVQLVQ | 120 |
| L-CDR3 | |
| SGAEVKKPGASVKVSCKASGYTFTSYGISWVRQAPGQGLEWMGWISAYNGNTNYAQKLQG | 180 |
| H-CDR1 H-CDR12 | |
| RVTMTTDTSTSTAYMELRSLRSDDTAVYYCARARGLGWGSDYWGQGTLVTVSSGSASAPT | 240 |
| H-CDR3 | |
| L | 241 |
| Anti-spike RBD Ab36 | |
| (SEQ ID NO: 42) | |
| ELVMTQSPSSVSASVGDRVTITCRASQAIGSWLAWYQQKPGKAPKLLISSASSLQSGVPS | 60 |
| L-CDR1 L-CDR2 | |
| RFSGSGSGTDFTLTISSLQPEDFATYYCQQLDSYPVTFGPGTKVDIKGGSSRSSEVQLLE | 120 |
| L-CDR3 | |
| SGGGLIQPGGSLRLSCAASGLTVSSNYMSWVRQAPGKGLEWVSVIYSGGSTYYADSVKGR | 180 |
| H-CDR1 H-CDR12 | |
| FTISRDNSKNTLYLQMNSLRAEDTAVYYCARAIDSGAFDIWGQGTMVTVSSGSASAPTL | 239 |
| H-CDR3 | |
| Anti-spike RBD Ab39 | |
| (SEQ ID NO: 43) | |
| ELVLTQSPGTLSLSPGERATLSCRASQSLYNNFLAWYQQKPGQAPRLLIYAASNRATGIP | 60 |
| L-CDR1 L-CDR2 | |
| DRFSGSGSGTDFTLTISRLEPEDFAVYYCQQYGSSPGYTFGQGTKLEIKGGSSRSSQVQL | 120 |
| L-CDR3 | |
| VQSGAEVKKPGSSVKVSCKASGGTFSSYAISWVRQAPGQGLEWMGGIIPIFGTANYAQKF | 180 |
| H-CDR1 H-CDR12 | |
| QGRVTITADESTSTAYMELSSLRSEDTAVYYCARTLRQYYYYGMDVWGQGTTVTVSSGSA | 240 |
| H-CDR3 | |
| SAPTL | 245 |
| Anti-spike RBD Ab37 | |
| (SEQ ID NO: 44) | |
| ELMLTOPPSVSVAPGQTARITCGGNNIGSKSVHWYQQKPGQAPVLVVYDDSDRPSGIPER | 60 |
| L-CDR1 L-CDR2 | |
| FSGSNSGNTATLTISRVEAGDEADYYCQVWDNTLLFGGGTELTVLGGGSSRSSGVQLVES | 120 |
| L-CDR3 | |
| GGGLVQPGGSLRLSCSASGFTFSSYAMHWVRQAPGKGLEYVSGISSNGGGTHYADSVKGR | 180 |
| H-CDR1 H-CDR12 | |
| FSISRDNSKNTLYLQMSSLRAEDTAVYYCVKDDSGSYFSLFDHWGQGTLVTVSSASTKGP | 240 |
| H-CDR3 | |
| SV | 242 |
| Anti-spike RBD Ab27 | |
| (SEQ ID NO: 45) | |
| ELQMTQSPSSLSASVGDRVTITCRASQSISSYLNWYQQKPGKAPKLLIYAASSLQSGVPS | 60 |
| L-CDR1 L-CDR2 | |
| RFSGSGSGTDFTLTISSLQPEDFATYYCQQANSFPFTFGPGTKVDIKGGSSRSSQVQLVQ | 120 |
| L-CDR3 | |
| SGAEVKKPGASVKVSCKASGYTFTSYGISWVRQAPGQGLEWMGWVNPNGGHTGYAQKFQG | 180 |
| H-CDR1 H-CDR12 | |
| RVTMTRHTATNTVYMELSSLTSDDTAVYYCARGKGYYYGMDVWGQGTTVLVSSASTKGPS | 240 |
| H-CDR3 | |
| V | 241 |

In some embodiments, a useful flexible linker is 1, 2, 3, 4, 5, 6, 7, 8, 9, or 10 repeats of the sequence GGGS (SEQ ID NO:34). In some embodiments, a useful flexible linker is 1, 2, 3, 4, 5, 6, 7, 8, 9, or 10 repeats of the sequence GGGGS (SEQ ID NO:35). In some embodiments, a useful flexible linker is 1, 2, 3, 4, 5, 6, 7, 8, 9, or 10 repeats of the sequence GGSSRSS (SEQ ID NO: 37)

A useful Spike CAR can consist of or comprise a mature CAR sequence or an immature CAR sequence (e.g., one having a signal sequence). The CAR can be expressed in a form that includes a signal sequence, e.g., a human GM-CSF receptor alpha signal sequence (MLLLVTSLLLCELPHPAFLLIP; SEQ ID NO:36). The CAR or polypeptide can be expressed with additional sequences that are useful for monitoring expression, for example, a T2A skip sequence and a truncated EGFRt or truncated CD19 (can consist of or comprise the amino acid sequence of SEQ ID NO:53-56) or low-affinity nerve growth factor receptor (LNGFR; SEQ ID NO: 48). Thus, the CAR can comprise or consist of the amino acid sequence of the full-length CAR (SEQ ID NOs: 49-52) or can comprise or consist of an amino acid sequence that is at least 95%, 96%, 97%, 98% or 99% identical to SEQ ID NOs: 49-52. The CAR or polypeptide can comprise or consist of the amino acid sequence of any of SEQ ID NOs: 49-52 with up to 1, 2, 3, 4 or 5 amino acid changes (preferably conservative amino acid changes). The CAR or polypeptide can comprise any of SEQ ID Nos: 1, 29-33, and 38-45.

(SEQ ID NO: 48)
MGAGATGRAMDGPRLLLLLLLGVSLGGAKEACPTGLYTHSGECCKACNL
GEGVAQPCGANQTVCEPCLDSVTFSDVVSATEPCKPCTECVGLQSMSAP
CVEADDAVCRCAYGYYQDETTGRCEACRVCEAGSGLVFSCQDKQNTVCE
ECPDGTYSDEANHVDPCLPCTVCEDTERQLRECTRWADAECEEIPGRWI
TRSTPPEGSDSTAPSTQEPEAPPEQDLIASTVAGVVTTVMGSSQPVVTR
GTTDNLIPVYCSILAAVVVGLVAYIAFKRWNSCKQNK

In some embodiments, the nucleic acid encoding amino acid sequence encoding the Spike CAR is codon optimized.

### Spacer Region

The CAR or polypeptide described herein can include a spacer located between the targeting domain (i.e., a Spike-protein targeted ScFv or variant thereof) and the transmembrane domain. A variety of different spacers can be used. Some of them include at least portion of a human Fc region, for example a hinge portion of a human Fc region or a CH3 domain or variants thereof. Table 1 below provides various spacers that can be used in the CARs described herein.

TABLE 1
Examples of Spacers

| Name | Length | Sequence |
|---|---|---|
| a3 | 3 aa | AAA |
| linker | 10 aa | GGGSSGGGSG (SEQ ID NO: 2) |
| IgG4 hinge (S→P) | 12 aa | ESKYGPPCPPCP (SEQ ID NO: 3) |
| (S228P) | | |
| IgG4 hinge | 12 aa | ESKYGPPCPSCP (SEQ ID NO: 4) |
| IgG1 hinge | 16 aa | LEPKSCDKTHTCPPCP (SEQ ID NO: 70) |
| IgG4 hinge (S228P) + | 22 aa | ESKYGPPCPPCPGGGSSGGGSG (SEQ ID NO: 5) |
| linker | | |
| CD28 hinge | 39 aa | IEVMYPPPYLDNEKSNGTIIHVKGKHLCPSPLFPGPSKP (SEQ ID NO: 6) |
| CD8 hinge-48 aa | 48 aa | AKPTTTPAPRPPTPAPTIASQPLSLRPEACRPAAGGAVHTRGLDFACD |
| | | (SEQ ID NO: 7) |
| CD8 hinge-45 aa | 45 aa | TTTPAPRPPTPAPTIASQPLSLRPEACRPAAGGAVHTRGLDFACD |
| | | (SEQ ID NO: 8) |
| IgG4 (HL-CH3) | 129 aa | ESKYGPPCPPCPGGGSSGGGSGGQPREPQVYTLPPSQEEMTKNQVSLTCL |
| Also called IgG4 (HL-ΔCH2) | | VKGFYPSDIAVEWESNGQPENNYKTTPPVLDSDGSFFLYSRLTVDKSRWQE |
| (includes S228P in hinge) | | GNVFSCSVMHEALHNHYTQKSLSLSLGK (SEQ ID NO: 9) |
| IgG4 (L235E, N297Q) | 229 aa | ESKYGPPCPSCPAPEFEGGPSVFLFPPKPKDTLMISRTPEVTCVVVDVSQED |
| | | PEVQFNWYVDGVEVHNAKTKPREEQFQSTYRVVSVLTVLHQDWLNGKEY |
| | | KCKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK (SEQ ID NO: 10) |
| IgG4 (S228P, L235E, N297Q) | 229 aa | ESKYGPPCPPCPAPEFEGGPSVFLFPPKPKDTLMISRTPEVTCVVVDVSQED |
| | | PEVQFNWYVDGVEVHNAKTKPREEQFQSTYRVVSVLTVLHQDWLNGKEY |
| | | KCKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK (SEQ ID NO: 11) |
| IgG4 (CH3) | 107 aa | GQPREPQVYTLPPSQEEMTKNQVSLTCLVKGFYPSDIAVEWESNGQPENN |
| Also called IgG4 (ΔCH2) | | YKTTPPVLDSDGSFFLYSRLTVDKSRWQEGNVFSCSVMHEALHNHYTQKSL |
| | | SLSLGK (SEQ ID NO: 12) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain (called CH3 or ΔCH2) or both a CH3 domain and a CH2 domain. The immunoglobulin derived sequences can include one or more amino acid modifications, for example, 1, 2, 3, 4 or 5 substitutions, e.g., substitutions that reduce off-target binding.

The hinge/linker region can also comprise an IgG4 hinge region having the sequence ESKYGPPCPSCP (SEQ ID NO:4) or ESKYGPPCPPCP (SEQ ID NO:3). The hinge/linger region can also comprise the sequence ESKYGPPCPPCP (SEQ ID NO:3) followed by the linker sequence GGGSSGGGSG (SEQ ID NO:2) followed by IgG4 CH3 sequence GQPREPQVYTLPPSQEEMTKNQVSLTCLVKGFYPSDIAVEWESNGQPENNYKTTPPV LDSDGSFFLYSRLTVDKSRWQEGNVFSCSVMHEALHNHYTQKSLSLSLGK (SEQ ID NO:12). Thus, the entire linker/spacer region can comprise the sequence: ESKYGPPCPPCPGGGSSGGGSGGQPREPQVYTLPPSQEEMTKNQVSLTCLVKGFYPS DIAVEWESNGQPENNYKTTPPVLDSDGSFFLYSRLTVDKSRWQEGNVFSCSVMHEA LHNHYTQKSLSLSLGK (SEQ ID NO:11). In some cases, the spacer has 1, 2, 3, 4, or 5 single amino acid changes (e.g., conservative changes) compared to SEQ ID NO:11. In some cases, the IgG4 Fc hinge/linker region that is mutated at two positions (L235E; N297Q) in a manner that reduces binding by Fc receptors (FcRs).

### Transmembrane Domain

A variety of transmembrane domains can be used in the. Table 2 includes examples of suitable transmembrane domains. Where a spacer region is present, the transmembrane domain (TM) is located carboxy terminal to the spacer region.

TABLE 2
Examples of Transmembrane Domains

| Name | Accession | Length | Sequence |
|---|---|---|---|
| CD3z | J04132.1 | 21 aa | LCYLLDGILFIYGVILTALFL (SEQ ID NO: 13) |
| CD28 | NM_006139 | 27 aa | FWVLVVVGGVLACYSLLVTVAFIIFWV (SEQ ID NO: 14) |
| CD28(M) | NM_006139 | 28 aa | MFWVLVVVGGVLACYSLLVTVAFIIFWV (SEQ ID NO: 15) |
| CD4 | M35160 | 22 aa | MALIVLGGVAGLLLFIGLGIFF (SEQ ID NO: 16) |
| CD8tm | NM_001768 | 21 aa | IYIWAPLAGTCGVLLLSLVIT (SEQ ID NO: 17) |
| CD8tm2 | NM_001768 | 23 aa | IYIWAPLAGTCGVLLLSLVITLY (SEQ ID NO: 18) |
| CD8tm3 | NM_001768 | 24 aa | IYIWAPLAGTCGVLLLSLVITLYC (SEQ ID NO: 19) |
| 41BB | NM_001561 | 27 aa | IISFFLALTSTALLFLLFFLTLRFSVV (SEQ ID NO: 20) |

### Costimulatory Domain

The costimulatory domain can be any domain that is suitable for use with a CD3ζ signaling domain. In some cases the co-signaling domain is a 4-1BB co-signaling domain that includes a sequence that is at least 90%, at least 95%, at least 98% identical to or identical to: KRGRKKLLYIFKQPFMRPVQTTQEEDGCSCRFPEEEEGGCEL (SEQ ID NO:24). In some cases, the 4-1BB co-signaling domain has 1, 2, 3, 4 of 5 amino acid changes (preferably conservative) compared to SEQ ID NO:24.

The costimulatory domain(s) are located between the transmembrane domain and the CD3ζ signaling domain. Table 3 includes examples of suitable costimulatory domains together with the sequence of the CD3ζ signaling domain.

TABLE 3
CD38 Domain and Examples of Costimulatory Domains

| Name | Accession | Length | Sequence |
|---|---|---|---|
| CD3ζ | J04132.1 | 113 aa | RVKFSRSADAPAYQQGQNQLYNELNLGRREEYDVLDKRRGR |
| | | | DPEMGGKPRRKNPQEGLYNELQKDKMAEAYSEIGMKGERR |
| | | | RGKGHDGLYQGLSTATKDTYDALHMQALPPR (SEQ ID |
| | | | NO: 21) |
| CD28 | NM_006139 | 42 aa | RSKRSRLLHSDYMNMTPRRPGPTRKHYQPYAPPRDFAAYRS |
| | | | (SEQ ID NO: 22) |
| CD28gg* | NM_006139 | 42 aa | RSKRSRGGHSDYMNMTPRRPGPTRKHYQPYAPPRDFAAYR |
| | | | S (SEQ ID NO: 23) |
| 41BB | NM_001561 | 42 aa | KRGRKKLLYIFKQPFMRPVQTTQEEDGCSCRFPEEEEGGCEL |
| | | | (SEQ ID NO: 24) |
| OX40 | | 42 aa | ALYLLRRDQRLPPDAHKPPGGGSFRTPIQEEQADAHSTLAKI |
| | | | (SEQ ID NO: 25) |

In various embodiments: the costimulatory domain is selected from the group consisting of: a costimulatory domain depicted in Table 3 or a variant thereof having 1-5 (e.g., 1 or 2) amino acid modifications, a CD28 costimulatory domain or a variant thereof having 1-5 (e.g., 1 or 2) amino acid modifications, a 4-1BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD3ζ signaling domain and a short linker consisting of 2-10, e.g., 3 amino acids (e.g., GGG) is can be positioned between the costimulatory domain and the CD3ζ signaling domain. Other useful costimulatory domains include: B4, DAP10, DAP12 and IL21R.

### CD3ζ Signaling Domain

The CD3ζ Signaling domain can be any domain that is suitable for use with a CD3ζ signaling domain. In some cases, the CD3ζ signaling domain includes a sequence that is at least 90%, at least 95%, at least 98% identical to or identical to: RVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQAL PPR (SEQ ID NO:21). In some cases, the CD3ζ signaling has 1, 2, 3, 4 of 5 amino acid changes (preferably conservative) compared to SEQ ID NO:21.

### Truncated EGFR or CD19

The CD3ζ signaling domain can be followed by a ribosomal skip sequence (e.g., LEGGGEGRGSLLTCGDVEENPGPR; SEQ ID NO:27) and a truncated EGFR having a sequence that is at least 90%, at least 95%, at least 98% identical to or identical to: LVTSLLLCELPHPAFLLIPRKVCNGIGIGEFKDSLSINATNIKHFKNCTSISGDLHILPVA FRGDSFTHTPPLDPQELDILKTVKEITGFLLIQAWPENRTDLHAFENLEIIRGRTKQHG QFSLAVVSLNITSLGLRSLKEISDGDVIISGNKNLCYANTINWKKLFGTSGQKTKIISN RGENSCKATGQVCHALCSPEGCWGPEPRDCVSCRNVSRGRECVDKCNLLEGEPREF VENSECIQCHPECLPQAMNITCTGRGPDNCIQCAHYIDGPHCVKTCPAGVMGENNTL VWKYADAGHVCHLCHPNCTYGCTGPGLEGCPTNGPKIP SIATGMVGALLLLLVVAL GIGLFM (SEQ ID NO:28). In some cases, the truncated EGFR has 1, 2, 3, 4 of 5 amino acid changes (preferably conservative) compared to SEQ ID NO:28. Alternatively the CD3ζ signaling domain can be followed by a ribosomal skip sequence (e.g., LEGGGEGRGSLLTCGDVEENPGPR; SEQ ID NO:27) and a truncated CD19R (also called CD19t) having a sequence that is at least 90%, at least 95%, at least 98% identical to or identical to:

(SEQ ID NO: 26)
MPPPRLLFFLLFLTPMEVRPEEPLVVKVEEGDNAVLQCLKGTSDGPTQQ
LTWSRESPLKPFLKLSLGLPGLGIHMRPLAIWLFIFNVSQQMGGFYLCQ
PGPPSEKAWQPGWTVNVEGSGELFRWNVSDLGGLGCGLKNRSSEGPSSP
SGKLMSPKLYVWAKDRPEIWEGEPPCVPPRDSLNQSLSQDLTMAPGSTL
WLSCGVPPDSVSRGPLSWTHVHPKGPKSLLSLELKDDRPARDMWVMETG
LLLPRATAQDAGKYYCHRGNLTMSFHLEITARPVLWHWLLRTGGWKVSA
VTLAYLIFCLCSLVGILHLQRALVLRRKR

An amino acid modification refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

In some cases, the Spike CAR can be produced using a retrovirus or lentivirus or other vectors in which the CAR open reading frame is followed by a T2A ribosome skip sequence and a sequence full length or soluble human IL-15 (codon optimized or unoptimized) fused with a signal peptide, e.g., IL-2 signal peptide (e.g., having the sequence MYRMQLLSCIALSLALVTNS: SEQ ID NO: 46). Thus, the vector can encode a soluble IL-15 having the sequence:

(SEQ ID NO: 47)
GIHVFILGCFSAGLPKTEANWVNVISDLKKIEDLIQSMHIDATLYTESD
VHPSCKVTAMKCFLLELQVISLESGDASIHDTVENLIILANNSLSSNGN
VTESGCKECEELEEKNIKEFLQSFVHIVQMFIN

The CAR or polypeptide described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform a suitable expression host cells, preferably an NK cells but do not exclude other cells such as T cells, dendritic cells, macrophages cells, hematopoietic stem cells, or subsets of each of these type cells from patients or from donors. The cell type can also be iPSC cells.

### Anti-SARS-CoV-2 Spike Protein Antibodies

In an aspect is provided an anti-SARS-CoV-2 spike antibody including a light chain variable domain and a heavy chain variable domain, wherein the light chain variable domain includes: a CDR L1 as set forth in SEQ ID NO:A1, a CDR L2 as set forth in SEQ ID NO:A2, and a CDR L3 as set forth in SEQ ID NO:A3; and wherein said heavy chain variable domain includes: a CDR H1 as set forth in SEQ ID NO:A4, a CDR H2 as set forth in SEQ ID NO:A5, a CDR H3 as set forth in SEQ ID NO:A6.

In embodiments, the light chain variable domain of the antibody includes the sequence of SEQ ID NO:A91.

In embodiments, the heavy chain variable domain of the antibody includes the sequence of SEQ ID NO:A92.

In another aspect is provided an anti-SARS-CoV-2 spike antibody including a light chain variable domain and a heavy chain variable domain, wherein said light chain variable domain includes: a CDR L1 as set forth in SEQ ID NO:A16, a CDR L2 as set forth in SEQ ID NO:A17, and a CDR L3 as set forth in SEQ ID NO:A18; and wherein said heavy chain variable domain includes: a CDR H1 as set forth in SEQ ID NO:A19, a CDR H2 as set forth in SEQ ID NO:A20, a CDR H3 as set forth in SEQ ID NO:A21

In embodiments, the light chain variable domain of the antibody includes the sequence of SEQ ID NO:A93.

In embodiments, the heavy chain variable domain of the antibody includes the sequence of SEQ ID NO:A94.

In another aspect is provided an anti-SARS-CoV-2 spike antibody including a light chain variable domain and a heavy chain variable domain, wherein said light chain variable domain includes: a CDR L1 as set forth in SEQ ID NO:A31, a CDR L2 as set forth in SEQ ID NO:A32, and a CDR L3 as set forth in SEQ ID NO:A33; and wherein said heavy chain variable domain includes: a CDR H1 as set forth in SEQ ID NO:A34, a CDR H2 as set forth in SEQ ID NO:A35, a CDR H3 as set forth in SEQ ID NO:A36.

In embodiments, the light chain variable domain of the antibody includes the sequence of SEQ ID NO:A95.

In embodiments, the heavy chain variable domain of the antibody includes the sequence of SEQ ID NO:A96.

In another aspect is provided an anti-SARS-CoV-2 spike antibody including a light chain variable domain and a heavy chain variable domain, wherein said light chain variable domain includes: a CDR L1 as set forth in SEQ ID NO:A46, a CDR L2 as set forth in SEQ ID NO:A47, and a CDR L3 as set forth in SEQ ID NO:A48; and wherein said heavy chain variable domain includes: a CDR H1 as set forth in SEQ ID NO:A49, a CDR H2 as set forth in SEQ ID NO:A50, a CDR H3 as set forth in SEQ ID NO:A51.

In embodiments, the light chain variable domain of the antibody includes the sequence of SEQ ID NO:A97.

In embodiments, the heavy chain variable domain of the antibody includes the sequence of SEQ ID NO:A98.

In another aspect is provided an anti-SARS-CoV-2 spike antibody including a light chain variable domain and a heavy chain variable domain, wherein said light chain variable domain includes: a CDR L1 as set forth in SEQ ID NO:A61, a CDR L2 as set forth in SEQ ID NO:A62, and a CDR L3 as set forth in SEQ ID NO:A63; and wherein said heavy chain variable domain includes: a CDR H1 as set forth in SEQ ID NO:A64, a CDR H2 as set forth in SEQ ID NO:A65, a CDR H3 as set forth in SEQ ID NO:A66.

In embodiments, the light chain variable domain of the antibody includes the sequence of SEQ ID NO:A99.

In embodiments, the heavy chain variable domain of the antibody includes the sequence of SEQ ID NO:A100.

In another aspect is provided an anti-SARS-CoV-2 spike antibody including a light chain variable domain and a heavy chain variable domain, wherein said light chain variable domain includes: a CDR L1 as set forth in SEQ ID NO:A76, a CDR L2 as set forth in SEQ ID NO:A77, and a CDR L3 as set forth in SEQ ID NO:A78; and wherein said heavy chain variable domain includes: a CDR H1 as set forth in SEQ ID NO:A79, a CDR H2 as set forth in SEQ ID NO:A80, a CDR H3 as set forth in SEQ ID NO:A81.

In embodiments, the light chain variable domain of the antibody includes the sequence of SEQ ID NO:A101.

In embodiments, the heavy chain variable domain of the antibody includes the sequence of SEQ ID NO:A102.

In embodiments, the antibody is produced in a cell. In embodiments, the antibody is produced in a bacterial cell. In embodiments, the antibody is produced in an E. coli cell. In embodiment, the antibody is produced in a yeast cell. In embodiments, the antibody is produced in mammalian cell. In embodiments, the antibody is produced in a human cell.

In embodiments, the antibody is a humanized antibody.

In embodiments, the antibody is a chimeric antibody.

In embodiments, the antibody is a Fab′ fragment.

In embodiments, the antibody antibody is a single chain antibody (scFv).

In embodiments, the light chain variable domain and said heavy chain variable domain of the antibody form part of a scFv.

In embodiments, the antibody is an IgG.

In embodiments, the antibody is an IgG1.

In embodiments, the antibody is capable of binding the SARS-CoV-2 spike protein.

In embodiments, the antibody is bound to the SARS-CoV-2 spike protein.

In embodiments, the SARS-CoV-2 spike protein forms part of a virus.

In another aspect is provided a pharmaceutical composition including a therapeutically effective amount of an antibody as disclosed herein and a pharmaceutically acceptable excipient.

In another aspect is provided a method of treating COVID-19 in a subject in need thereof, said method including administering to a subject a therapeutically effective amount of an antibody as disclosed herein, thereby treating COVID-19 in said subject.

In another aspect is provided a method of treating a SARS-CoV-2 infection in a subject in need thereof, said method including administering to a subject a therapeutically effective amount of an antibody as disclosed herein, thereby treating said SARS-CoV-2 infection in said subject.

In embodiments, the pharmaceutical composition is a pharmaceutical preparation.

In embodiments, the treatment is a prophylactic treatment for COVID-19. In embodiments, the treatment is a prophylactic treatment for a SARS-CoV-2 infection.

In embodiments, the antibodies provided herein produce an immune response when administered to the subject.

In embodiments, the antibodies provided herein inhibit ACE2 enzymatic activity. In embodiments, the antibodies provided herein are inhibitors of ACE2 enzymatic activity.

In embodiments the subject is a patient (e.g. a human patient). In embodiments, the patient is at high risk of developing COVID-19. In embodiments, the patient is at high risk of dying from COVID-19. In embodiments, the patient at high risk is older than 65. In embodiments, the patient at high risk has diabetes. In embodiments, the patient at high risk has moderate asthma. In embodiments, the patient at high risk has severe asthma. In embodiments, the patient at high risk has chronic obstructive pulmonary disease. In embodiments, the patient at high risk has pulmonary fibrosis. In embodiments, the patient at high risk has cystic fibrosis. In embodiments, the patient at high risk has hypertension. In embodiments, the patient at high risk has diabetes mellitus. In embodiments, the patient at high risk has a cardiovascular disease such as, but limited to, coronary artery diseases, stroke, heart failure, hypertensive heart disease, rheumatic heart disease, or cardiomyopathy. In embodiments, the subject displays one or more symptoms of COVID-19. In embodiments, the subject has COVID-19.

In embodiments, the antibodies provided herein have a Kd to the SARS-CoV-2 spike protein of less than: 1000 micromolar. 500 micromolar. 250 micromolar. 125 micromolar. 50 micromolar. 25 micromolar. 10 micromolar. 5 micromolar. 2 micromolar, 1 micromolar, 500 nanomolar. 250 nanomolar, 125 nanomolar. 50 nanomolar. 25 nanomolar. In embodiments, the antibodies provided herein have a Kd to the SARS-CoV-2 spike protein of about 10 nanomolar. In embodiments, the antibodies provided herein have a Kd to the SARS-CoV-2 spike protein of about 5 nanomolar.

In embodiments, the antibodies provided herein have a Kd to the SARS-CoV-2 spike protein RBD domain that is at least 50%, 60%, 70%, 80%, 90%, or 100% less than to a SARS-CoV-2 spike protein without an RBD domain.

In embodiments, the antibodies provided herein have a Kd to the SARS-CoV-2 spike protein RBD domain that is at least 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, or 1000-fold less than to a SARS-CoV-2 spike protein without an RBD domain.

In an aspect is provided a nucleic acid encoding any of the antibodies provided herein including embodiments thereof. In embodiments, the nucleic acid is a plasmid. In embodiments, the nucleic acid is a double stranded nucleic acid. In embodiments, the nucleic is a single stranded nucleic acid. In embodiments the nucleic acid is DNA molecule. In embodiments, the nucleic acid is an RNA molecule. In embodiments, the nucleic acid includes the sequence of any of SEQ ID NO:A103, SEQ ID NO:A104, SEQ ID NO:A105, SEQ ID NO:A106, SEQ ID NO:A107, SEQ ID NO:A108

In embodiments, the antibody includes any of the sequences of SEQ ID NO:A15, SEQ ID NO:A30, SEQ ID NO:A45, SEQ ID NO:A60, SEQ ID NO:A75, SEQ ID NO:A90.

In an aspect is provided and antibody comprising: a light chain variable domain that is 99%, 98%, 95%, 94%, 93%, 92%, 91% or 90% identical SEQ ID NO:A91, wherein the light chain variable domain includes: a CDR L1 as set forth in SEQ ID NO:A1, a CDR L2 as set forth in SEQ ID NO:A2, and a CDR L3 as set forth in SEQ ID NO:A3; and heavy chain variable domain that is 99%, 98%, 95%, 94%, 93%, 92%, 91% or 90% identical SEQ ID NO:A92 and wherein the heavy chain variable domain includes: a CDR H1 as set forth in SEQ ID NO:A4, a CDR H2 as set forth in SEQ ID NO:A5, a CDR H3 as set forth in SEQ ID NO:A6

In an aspect is provided an antibody comprising: a light chain variable domain that is 99%, 98%, 95%, 94%, 93%, 92%, 91% or 90% identical SEQ ID NO:A93, wherein the light chain variable domain includes: a CDR L1 as set forth in SEQ ID NO:A16, a CDR L2 as set forth in SEQ ID NO:A17, and a CDR L3 as set forth in SEQ ID NO:A18; and a heavy chain variable domain that is 99%, 98%, 95%, 94%, 93%, 92%, 91% or 90% identical SEQ ID NO:A94 wherein the heavy chain variable domain includes: a CDR H1 as set forth in SEQ ID NO:A19, a CDR H2 as set forth in SEQ ID NO:A20, a CDR H3 as set forth in SEQ ID NO:A21. In an aspect is provided an antibody comprising: a light chain variable domain that is 99%, 98%, 95%, 94%, 93%, 92%, 91% or 90% identical SEQ ID NO:A95, wherein the light chain variable domain includes: a CDR L1 as set forth in SEQ ID NO:A31, a CDR L2 as set forth in SEQ ID NO:A32, and a CDR L3 as set forth in SEQ ID NO:A33; and a heavy chain variable domain that is 99%, 98%, 95%, 94%, 93%, 92%, 91% or 90% identical SEQ ID NO:A96, wherein the heavy chain variable domain includes: a CDR H1 as set forth in SEQ ID NO:A34, a CDR H2 as set forth in SEQ ID NO:A35, a CDR H3 as set forth in SEQ ID NO:A36.

In an aspect is provided an antibody comprising: a light chain variable domain that is 99%, 98%, 95%, 94%, 93%, 92%, 91% or 90% identical SEQ ID NO:A97, wherein the light chain variable domain includes: a CDR L1 as set forth in SEQ ID NO:A46, a CDR L2 as set forth in SEQ ID NO:A47, and a CDR L3 as set forth in SEQ ID NO:A48; and a heavy chain variable domain that is 99%, 98%, 95%, 94%, 93%, 92%, 91% or 90% identical SEQ ID NO:A98, wherein the heavy chain variable domain includes: a CDR H1 as set forth in SEQ ID NO:A49, a CDR H2 as set forth in SEQ ID NO:A50, a CDR H3 as set forth in SEQ ID NO:A51.

In an aspect is provided an antibody comprising: a light chain variable domain that is 99%, 98%, 95%, 94%, 93%, 92%, 91% or 90% identical SEQ ID NO:A99, wherein the light chain variable domain includes: a CDR L1 as set forth in SEQ ID NO:A61, a CDR L2 as set forth in SEQ ID NO:A62, and a CDR L3 as set forth in SEQ ID NO:A63; and a heavy chain variable domain that is 99%, 98%, 95%, 94%, 93%, 92%, 91% or 90% identical SEQ ID NO:A100, wherein the heavy chain variable domain includes: a CDR H1 as set forth in SEQ ID NO:A64, a CDR H2 as set forth in SEQ ID NO:A65, a CDR H3 as set forth in SEQ ID NO:A66.

In an aspect is provided an antibody comprising: a light chain variable domain that is 99%, 98%, 95%, 94%, 93%, 92%, 91% or 90% identical SEQ ID NO:A101, wherein the light chain variable domain includes: a CDR L1 as set forth in SEQ ID NO:A76, a CDR L2 as set forth in SEQ ID NO:A77, and a CDR L3 as set forth in SEQ ID NO:A78; and a heavy chain variable domain that is 99%, 98%, 95%, 94%, 93%, 92%, 91% or 90% identical SEQ ID NO:A102, wherein the heavy chain variable domain includes: a CDR H1 as set forth in SEQ ID NO:A79, a CDR H2 as set forth in SEQ ID NO:A80, a CDR H3 as set forth in SEQ ID NO:A81.

In an aspect is provided a cell including a nucleic acid encoding an antibody as provided herein including embodiments thereof. In embodiments, the cell is a bacterial cell. In embodiments, the cell is an E. coli cell. In embodiments, the cell is a yeast cell. In embodiments, the cell is a mammalian cell. In embodiments, the cell is a human cell.

### Mutant ACE2 Protein

In an aspect is provided a recombinant ACE2 protein including an ACE2 extracellular domain, wherein said ACE2 extracellular domain includes an amino acid mutation that decreases enzymatic activity relative to an ACE2 protein without said amino acid mutation, and wherein the recombinant ACE2 protein binds to a SARS-CoV-2 Spike protein with a Kd of less than or equal to that of an ACE2 protein without said amino acid mutation.

In an aspect is provided a nucleic acid encoding an ACE2 protein of SEQ ID NO:B2 or SEQ ID NO:B4, e.g., amino acids 18-740 of SEQ ID NO:B2 or amino acids 18-615 of SEQ ID NO:B4, wherein the ACE2 protein includes an amino acid substitution at a position corresponding to position 273 or position 345 or both 273 and 345. In embodiments, the substitution at position 273 is a R273S substitution. In embodiments, the substitution at position 345 is a H345F or H345S substitution. In embodiments, the substitution at position 345 is a H345F substitution. In embodiments, the substitution at position 345 is a H345S substitution. In embodiments, the nucleic acid include SEQ ID NO:B5. In embodiments, the nucleic acid include SEQ ID NO:B7. In embodiments, the nucleic acid include SEQ ID NO:B9. In embodiments, the nucleic acid include SEQ ID NO:B15. In embodiments, the nucleic acid include SEQ ID NO:B17. In embodiments, the nucleic acid include SEQ ID NO:B19.

In embodiments, the ACE2 protein as provided herein including embodiments thereof is a full length ACE2 of SEQ ID NO:B2. In embodiments, the ACE2 protein as provided herein including embodiments thereof is a full length ACE2. In embodiments, the ACE2 protein as provided herein including embodiments thereof includes SEQ ID NO:B2 or amino acids 18-740 thereof. In embodiments, the ACE2 protein as provided herein including embodiments thereof is a short-length ACE2 protein. In embodiments, the ACE2 protein as provided herein including embodiments thereof is a short length ACE2 protein. In embodiments, the ACE2 protein as provided herein including embodiments thereof includes the sequence of SEQ ID NO:B4 or amino acids 18-615 thereof. In various embodiments, the fragment lacks the first 17, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205 or 210 amino acids of SEQ ID NO:B2 or SEQ ID NO:B4. In various embodiments, the fragment lacks the last 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205 or 210 amino acids of SEQ ID NO:B2 or SEQ ID NO:B4.

In embodiments, the ACE2 protein as provided herein including embodiments thereof is not a full length protein.

In embodiments, the protein is a fusion protein comprising amino acids 18-740 of SEQ ID NO:B6, SEQ ID NO:B8 or SEQ ID NO:B10 followed by SEQ ID NO:B12 or SEQ ID NO:B14. In embodiments, the protein is a fusion protein comprising amino acids 18-615 of SEQ ID NO:B16, SEQ ID NO:B18 or SEQ ID NO:B20 followed by SEQ ID NO:B12 or SEQ ID NO:B14.

In an aspect is provided a cell expressing a nucleic acid as provided herein including embodiments thereof.

In embodiments, the protein is soluble in an aqueous liquid.

In embodiments, the protein is soluble in human blood.

In embodiments, the protein does not include an ACE2 transmembrane domain.

In embodiments, the recombinant ACE2 protein binds to a SARS-CoV-2 Spike protein with a Kd of less than 1 nM. In embodiments, the recombinant ACE2 protein binds to a SARS-CoV-2 Spike protein with a Kd of less than 2 nM. In embodiments, the recombinant ACE2 protein binds to a SARS-CoV-2 Spike protein with a Kd of less than 5 nM. In embodiments, the recombinant ACE2 protein binds to a SARS-CoV-2 Spike protein with a Kd of less than 10 nM. In embodiments, the recombinant ACE2 protein binds to a SARS-CoV-2 Spike protein with a Kd of less than 25 nM. In embodiments, the recombinant ACE2 protein binds to a SARS-CoV-2 Spike protein with a Kd of less than 50 nM. In embodiments, the recombinant ACE2 protein binds to a SARS-CoV-2 Spike protein with a Kd of less than 100 nM. In embodiments, the recombinant ACE2 protein binds to a SARS-CoV-2 Spike protein with a Kd of less than 1000 nM. In embodiments, the recombinant ACE2 protein binds to a SARS-CoV-2 Spike protein with a Kd of less than 10,000 nM.

In embodiments, the recombinant ACE2 protein binds to the SARS-CoV-2 Spike protein with a Kd less than or equal to that of a ACE2 protein of SEQ ID NO:B2. In embodiments, the recombinant ACE2 protein binds to the SARS-CoV-2 Spike protein with a Kd less than to that of a ACE2 protein of SEQ ID NO:B2. In embodiments, the recombinant ACE2 protein binds to the SARS-CoV-2 Spike protein with a Kd less equal to that of a ACE2 protein of SEQ ID NO:B2.

In embodiments, the recombinant ACE2 protein includes SEQ ID NO:B1 or functional fragment thereof, wherein the recombinant ACE2 protein includes an amino acid mutation at a position equivalent to position 273 or position 345 of SEQ ID NO:B2 or SEQ ID NO:B4. In embodiments, the recombinant ACE2 protein includes SEQ ID NO:B1 or functional fragment thereof, wherein the recombinant ACE2 protein includes an amino acid substitution at a position corresponding to position 273 or position 345 of SEQ ID NO:B2 or SEQ ID NO:B4.

In embodiments, the amino acid substitution is at a position equivalent to position 273. In embodiments, the amino acid substitution is at a position corresponding to position 273. In embodiments, the amino acid substitution at a position corresponding to position 273 is an R to S amino acid substitution.

In embodiments, the amino acid substitution is a R273 S substitution.

In embodiments, the sequence of the recombinant ACE2 protein is SEQ ID NO:B6. In embodiments, the sequence of the recombinant ACE2 protein comprises at least 200, 250, 300, 350, 400, 450, 500, 550, 600, 650 or 700 consecutive amino acids of SEQ ID NO:B6. In embodiments, the sequence of the recombinant ACE2 protein is SEQ ID NO:B8. In embodiments, the sequence of the recombinant ACE2 protein comprises at least 200, 250, 300, 350, 400, 450, 500, 550, 600, 650 or 700 consecutive amino acids of SEQ ID NO:B8. In embodiments, the sequence of the recombinant ACE2 protein is SEQ ID NO:B10. In embodiments, the sequence of the recombinant ACE2 protein comprises at least 200, 250, 300, 350, 400, 450, 500, 550, 600, 650 or 700 consecutive amino acids of SEQ ID NO:B10.

In embodiments, the sequence of the recombinant ACE2 protein is SEQ ID NO:B16. In embodiments, the sequence of the recombinant ACE2 protein comprises at least 200, 250, 300, 350, 400, 450, 500, 550 or 600 consecutive amino acids of SEQ ID NO:B16. In embodiments, the sequence of the recombinant ACE2 protein is SEQ ID NO:B8. In embodiments, the sequence of the recombinant ACE2 protein comprises at least 200, 250, 300, 350, 400, 450, 500, 550 or 600 consecutive amino acids of SEQ ID NO:B18. In embodiments, the sequence of the recombinant ACE2 protein is SEQ ID NO:B20. In embodiments, the sequence of the recombinant ACE2 protein comprises at least 200, 250, 300, 350, 400, 450, 500, 550 or 600 consecutive amino acids of SEQ ID NO:B20.

In embodiments, the amino acid substitution is at a position equivalent to position 345. In embodiments, the amino acid substitution is at a position corresponding to position 345. In embodiments, the amino acid substitution at a position corresponding to position 345 is an H to F amino acid substitution.

In embodiments, the amino acid substitution is a H345F substitution.

In embodiments, the amino acid substitution is a H345S substitution. In embodiments, the amino acid substitution at a position corresponding to position 345 is an H to S amino acid substitution.

In an aspect is provided a pharmaceutical composition including a recombinant ACE2 protein as disclosed herein including embodiments thereof and a pharmaceutically acceptable carrier.

In embodiments, the pharmaceutically acceptable carrier is Arginine HCl NaCl, and sucrose. In embodiments, the Arginine HCl concentration is 0.025 mmol per mL. In embodiments, the NaCl concentration is 0.12 mmol per mL. In embodiments, the sucrose concentration is 10 mg per mL. Arginine HCl NaCl, and sucrose. In embodiments, the Arginine HCl concentration is about 0.025 mmol per mL. In embodiments, the NaCl concentration is about 0.12 mmol per mL. In embodiments, the sucrose concentration is about 10 mg per mL. In embodiments, the Arginine HCl concentration is less than 0.025 mmol per mL. In embodiments, the NaCl concentration is less than 0.12 mmol per mL. In embodiments, the sucrose concentration is less than 10 mg per mL. In embodiments, the Arginine HCl concentration is more than 0.025 mmol per mL. In embodiments, the NaCl concentration is more than 0.12 mmol per mL. In embodiments, the sucrose concentration is more than 10 mg per mL.

In embodiments, the pharmaceutical composition includes a mutated recombinant ACE2 protein as provided herein including embodiments thereof, wherein the mutated recombinant ACE2 protein is fused with the gene of the Fc component of a human IgG2, IgG4, or IgG5 antibody. In embodiments, the Fc component of the fusion protein includes the CH1, CH2 and CH3 domains and hinge regions of the IgG2, IgG4 or IgG5 antibody.

In an aspect is provided a method of treating or preventing COVID-19 in a subject in need thereof, the method including administering an effective amount of a recombinant ACE2 protein as disclosed herein including embodiments thereof to the subject or an effective amount of a pharmaceutical composition as disclosed herein including embodiments thereof to the subject.

In an aspect is provided a method of treating or preventing a SARS-CoV-2 infection in a subject in need thereof, the method including administering an effective amount of a recombinant ACE2 protein as disclosed herein including embodiments thereof to the subject or an effective amount of a pharmaceutical composition as disclosed herein including embodiments thereof to the subject. In embodiments the subject is a patient (e.g. a human patient). In embodiments, the patient is at high risk or developing COVID-19. In embodiments, the patient is at high risk of dying from COVID-19. In embodiments, the patient at high risk is older than 65. In embodiments, the patient at high risk has diabetes. In embodiments, the patient at high risk has moderate asthma. In embodiments, the patient at high risk has severe asthma. In embodiments, the patient at high risk has chronic obstructive pulmonary disease. In embodiments, the patient at high risk has pulmonary fibrosis. In embodiments, the patient at high risk has cystic fibrosis. In embodiments, the patient at high risk has hypertension. In embodiments, the patient at high risk has diabetes mellitus. In embodiments, the patient at high risk has a cardiovascular disease such as, but limited to, coronary artery diseases, stroke, heart failure, hypertensive heart disease, rheumatic heart disease, or cardiomyopathy. In embodiments, the subject displays one or more symptoms of COVID-19. In embodiments, the subject has COVID-19.

In embodiments, the recombinant ACE2 protein provided herein including embodiments thereof inhibits the binding of the SARS-CoV-2 spike protein to the naturally occurring ACE2 protein (e.g. at the membrane of a cell). Therefore, in embodiments, the recombinant ACE2 protein provided herein inhibits the entry of SARS-CoV-2 into the cell (e.g. a human cell). In embodiments, the recombinant ACE2 protein provided herein including embodiments thereof is an inhibitor of SARS-CoV-2 infection in a subject (e.g., a human subject).

In embodiments, the recombinant ACE2 protein provided herein including embodiments thereof is used as a prophylactic treatment for COVID-19 in a subject (e.g. a human subject). In embodiments, the recombinant ACE2 protein provided herein including embodiments thereof is used as a treatment for COVID-19 in a subject (e.g. a human subject). In embodiments, the recombinant ACE2 protein provided herein including embodiments thereof is used in a treatment preventing a SARS-CoV-2 infection in a subject(e.g. a human subject). In embodiments, the recombinant ACE2 protein provided herein including embodiments thereof is used in a treatment for SARS-CoV-2 infection in a subject (e.g. a human subject).

### Definitions

As used herein, the singular forms “a”, “an” and “the” are intended to include the plural forms as well, unless the context clearly indicates otherwise.

In this disclosure, “comprises,” “comprising,” “containing” and “having” and the like can have the meaning ascribed to them in U.S. Patent law and can mean “ includes,” “including,” and the like. “Consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
Alanine (A), Glycine (G);
Aspartic acid (D), Glutamic acid (E);
Asparagine (N), Glutamine (Q);
Arginine (R), Lysine (K);
Isoleucine (I), Leucine (L), Methionine (M), Valine (V);
Phenylalanine (F), Tyrosine (Y), Tryptophan (W);
Serine (S), Threonine (T); and
Cysteine (C), Methionine (M)
(see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. For example, a selected residue in a selected antibody (or Fab domain) corresponds to light chain threonine at Kabat position 40, when the selected residue occupies the same essential spatial or other structural relationship as a light chain threonine at Kabat position 40. In some embodiments, where a selected protein is aligned for maximum homology with the light chain of an antibody (or Fab domain), the position in the aligned selected protein aligning with threonine 40 is said to correspond to threonine 40. Instead of a primary sequence alignment, a three dimensional structural alignment can also be used, e.g., where the structure of the selected protein is aligned for maximum correspondence with the light chain threonine at Kabat position 40, and the overall structures compared. In this case, an amino acid that occupies the same essential position as threonine 40 in the structural model is said to correspond to the threonine 40 residue.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab₂, bispecific Fab₂, trispecific Fab₃, monovalent IgGs, scFv, bispecific antibodies, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable heavy chain provided herein includes in N-terminal to C-terminal direction a CDR H1, a CDR H2 and a CDR H3.

The terms “FR L1”, “FR L2”, “FR L3” and “FR L4” as provided herein are used according to their common meaning in the art and refer to the framework regions (FR) 1, 2, 3 and 4 of the variable light (L) chain of an antibody. In embodiments, the variable light chain provided herein includes in N-terminal to C-terminal direction a FR L1, a FR L2, a FR L3 and a FR L4. Likewise, the terms “FR H1”, “FR H2”, “FR H3” and “FR H4” as provided herein are used according to their common meaning in the art and refer to the framework regions (FR) 1, 2, 3 and 4 of the variable heavy (H) chain of an antibody. In embodiments, the variable heavy chain provided herein includes in N-terminal to C-terminal direction a FR H1, a FR H2, a FR H3 and a FR H4.

The terms “KD”,“Kd”, “K_{D}” or “K_{d}” are used according to its commonly known meaning in the art. A dissociation constant is a specific type of equilibrium constant that measures the propensity of a larger object to separate (dissociate) reversibly into smaller components, as when a complex falls apart into its component molecules, or when a salt splits up into its component ions. The dissociation constant is the inverse of the association constant. KD is the equilibrium dissociation constant, a ratio of k_{off}/kₒₙ, between the antibody and its antigen. KD and affinity are inversely related. The KD value relates to the concentration of antibody (the amount of antibody needed for a particular experiment) and so the lower the KD value (lower concentration) and thus the higher the affinity of the antibody.

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “spike protein”, “S protein”, or “SARS-CoV-2 S protein” are used in accordance with their plain meaning as understood in the art and refer to the spike (S) protein of the SARS-CoV-2, or variants or homologs thereof. The spike (S) protein of the SARS-CoV-2 includes any of the recombinant or naturally-occurring forms of the spike (S) protein of the SARS-CoV-2, or variants or homologs thereof, that maintain S protein activity (e.g. within at least 50%, 80%, 90%, 95%, 96%, 97%, 98%, 99% or 100% activity compared to the S protein). In embodiments, the S protein is a large (approx. 180 kDa) glycoprotein. In embodiments, the S protein is present on the viral surface as a trimer. The S protein may include two domains, S1 and S2. In embodiments, the S1 domain mediates receptor binding and is divided into two sub-domains, with the N-terminal subdomain (NTD) often binding sialic acid and the C-terminal subdomain (also known as C-domain) binding a specific proteinaceous receptor. In embodiments, the S2 domain mediates viral-membrane fusion through the exposure of a highly conserved fusion peptide. The fusion peptide may be activated through proteolytic cleavage at a site found immediately upstream (S2′), which is common to all coronaviruses. In many (but not all) coronaviruses, additional proteolytic priming may occur at a second site located at the interface of the S1 and S2 domains (S1/S2). In some embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring the S protein polypeptide (e.g. YP_009724390.1). In embodiments, the S protein is the protein as identified by the NCBI sequence reference YP_009724390.1, homolog or functional fragment thereof.

The term “ACE2” or “angiotensin converting enzyme 2” as referred to herein are used in accordance with their plain meaning as understood in the art and refer to any of the recombinant or naturally-occurring forms of the ACE2 enzyme, or variants or homologs thereof that maintain ACE2 enzyme activity. Wild-type ACE2 protein is substantially identical to the protein identified by the UniProt reference number Q9BYF1 or a variant or homolog having substantial identity thereto. ACE2 is typically a zinc containing metalloenzyme which catalyzes the conversion of angiotensin II (Ang 1-8) to angiotensin (Ang 1-7), which is a vasodilator. ACE2 also is the cellular receptor for sudden acute respiratory syndrome (SARS) coronavirus/SARS-CoV and human coronavirus NL63/HCoV-NL63.

The terms “SARS-CoV-2” or “SARS-CoV 2” refer to the Severe Acute Respiratory Syndrome Coronavirus 2. In embodiments, the SARS-CoV-2 is the strain of coronavirus that causes coronavirus disease 2019 (COVID-19), the respiratory illness responsible for the COVID-19 pandemic. In embodiments, the SARS-CoV-2 is colloquially known as simply the coronavirus, it was previously referred to by its provisional name, 2019 novel coronavirus (2019-nCoV), and has also been called human coronavirus 2019 (HCoV-19 or hCoV-19). In embodiments, the SARS-CoV-2 is a Baltimore class IV positive-sense single-stranded RNA virus that is contagious in humans.

The terms “COVID19”, “COVID-19” refer to the coronavirus disease 2019, caused by SARS-CoV-2. In embodiments, the COVID-19 is a respiratory illness characterized by symptoms such as fever, cough, loss of appetite, fatigue, shortness of breath, coughing up sputum, muscle aches and pains, nausea, vomiting, diarrhea, sneezing, runny nose, sore throat, skin lesions, chest tightness, palpitations, decrease sense or loss of sense of smell, and/or disturbances in sense of taste. Comorbidities of COVID-19 include moderate or severe asthma, pre-existing chronic obstructive pulmonary disease, pulmonary fibrosis, cystic fibrosis, hypertension, diabetes mellitus, and cardiovascular diseases such as, but limited to, coronary artery diseases, stroke, heart failure, hypertensive heart disease, rheumatic heart disease, or cardiomyopathy.

The term “prophylactic treatment” as used herein, refers to any intervention using the compositions embodied herein, that is administered to an individual in need thereof or having an increased risk of acquiring a respiratory tract infection, wherein the intervention is carried out prior to the onset of a viral infection, e.g. SARS-CoV-2, and typically has in effect that either no viral infection occurs or no clinically relevant symptoms of a viral infection occur in a healthy individual no[0000] upon subsequent exposure to an amount of infectious viral agent that would otherwise, i.e. in the absence of such a prophylactic treatment, be sufficient to cause a viral infection.

Any compositions or methods provided herein can be combined with one or more of any of the other compositions and methods provided herein.

A number of embodiments have been described. Nevertheless, it will be understood that various modifications may be made. Accordingly, other embodiments are within the scope of the claims.

Other features and advantages of the described compositions and methods will be apparent from the following detailed description and figures, and from the claims.

### DESCRIPTION OF DRAWINGS

FIG. 1: Expression of CAR in retrovirally transduced NK and T cells, determined by flow cytometry using a goat anti-mouse F(ab′)2 polyclonal antibody. Data shown are representative of three donors with similar transduction efficiency.

FIGS. 2A-2C: (A) Expression of CAR-transduced NK cells determined by flow cytometry using a goat anti-mouse F(ab′)2 polyclonal antibody. Low-affinity nerve growth factor receptor (LNGFR) with a minimum affinity to binding with NGF was used as a marker to define successfully transduced cells and enrich CAR NK cells in the manufacturing process when it is necessary. (B) Enumeration of expanded CAR NK cells. (C) CAR NK cells were sorted to over 98% purity and CAR expression was determined 1 and 2 weeks after being in co-culture, assessed by flow cytometry.

FIGS. 3A-3C: Absolute cell number of lymphocyte subsets in COVID-19 patients at indicated time. PBMCs were isolated from COVID-19 patients and analyzed by flow cytometry. HD: healthy donor; PD: patient donor. Day 1 indicates 7 days after the patient shows symptom of COVID-19 and positive for viral infection detected by PCR. HD: healthy donor; PD: patient donor. Days 7, 14, and 21 indicates 7, 14, and 21 days after patients showed symptom of COVID-19 and positive PCR detection.

FIG. 4: Engineered stem-like cord blood NK cells expressing firefly luciferase, m15-NK, m15c-NK, s15-NK, and s15c-NK were expanded for 2 weeks and were injected intravenously into NSG mice at the dose of 12×10⁶/mice. Control group received PBS. Bioluminescence imaging showing distribution of firefly luciferase expressing NK cells after 1, 2, 4 and 6 days.

FIGS. 5A-5C: Engineered stem-like cord blood NK cells expressing firefly luciferase were expanded for 2 weeks and were injected intravenously into NSG mice at the dose of 12×10⁶/mice. Control group received PBS. (A) Bioluminescence imaging showing distribution of firefly luciferase expressing NK cells after 24 hrs. (B) Absolute count of total NK cells, quantified by flow cytometry, in different organs of mice injected with PBS and NK cells after 24 hrs of injection. Data regarding percentages of human NK cells among mouse mononuclear cells are similar (not shown). (C) Representative flow cytometry for the expression of human CD45 gated on live cells in different organs. **P<0.01.

FIG. 6: Cell counts of NK cell numbers, CD8 cells, B cells, and CD4 T cells at days 7, 14, and 21 to show immune response in twelve COVID-19 patients.

FIGS. 7A-7B: (A) Real-time cytolysis of four different CAR constructs against SPIKE-expressed H23cell line (n=4). LNGFR is serving as a control vector. Cytolysis is normalized to the tumor only group. (B) Cytolysis analyzed at the time point of 48 hours post coculturing with CAR NK and SPIKE-expressed cells (N=4). SPIKE L1H1 and SPIKE H1L1 are anti-Spike antibody derived CAR NK cells with light chain followed by heavy chain or vice versa, respectively. ACE2-L and ACE2-S are two NK CARs with long (L) and short (S) form (p<0.01. ACE2 CAR vs LNGFR).

FIG. 7: The growth of Spike protein-expressing cells was monitored using Real-Time Cell Analysis. All four CAR targeted to SAR-COV-2 slowed the expansion of the Spike protein-expressing cells.

FIGS. 8A-8D: Generation of mACE2-CAR_sIL-15 natural killer cells. (A) Schematic design of the mACE2-CAR and sIL-15 retroviral vectors. The mACE2-CAR vector comprises a human mutated ACE2, a human IgG1-hinge region, and CD28 transmembrane region, followed by the intracellular domains of CD28 and CD3ζ linked to a T2A and a truncated (t) LNGFR (a). The sIL-15 vector comprises an IL-2 signaling peptide, a codon optimized IL-15, and a CSF2R signaling peptide, followed by a truncated (t) EGFR (b). (B) Generation of mACE2-CAR_sIL-15 natural killer (NK) cells. (C) The transduction efficiency of control tEGFR or sIL-15 NK cells and mACE2-CAR_sIL-15 NK cells was determined by flow cytometry. Control tEGFR or sIL-15 NK cells expressed the tEGFR marker, while mACE2-CAR_sIL-15 NK cells expressed the tLNGFR maker, representing mACE2-CAR expression, as well as the tEGFR marker, representing sIL-15 expression. (D) Activated and expanded mACE2-CAR_sIL-15 NK cells exhibited a phenotype similar to that of control activated and expanded NK cells transduced with only tEGFR or sIL-15. Representative flow cytometry plots and summary data from 3 different donors are shown.

FIGS. 9A-9G: mACE2-CAR_sIL-15 NK cells bind to SARS-CoV-2 spike protein and VSV-SARS-CoV-2 chimeric viral particles. (A) Schematic illustration of mACE2-CAR_sIL-15 NK cells binding to spike S1-His-tagged recombinant protein. MFI=mean fluorescence intensity. (B, C) Representative flow cytometry plots showed the results of mACE2-CAR_sIL-15 NK cells binding to spike S1-His-tagged recombinant protein. LNGFR was used as a marker for mACE2-CAR-positive NK cells. Summary data from 5 different donors are shown in (C). (D) Schematic illustration of spike S1-His-tagged recombinant protein binding to VSV-SARS-CoV-2 chimeric viral particles, which are recognized by anti-spike and its corresponding fluor-conjugated secondary. (E) Representative flow cytometry plots showed the efficiency of control NK or mACE2-CAR_sIL-15 NK cells binding to VSV-SARS-CoV-2 chimeric viral particles. (F, G) Representative flow cytometry plots showed the efficiency of CAR-negative or CAR-positive subsets in mACE2-CAR_sIL-15 NK cells binding to VSV-SARS-CoV-2 chimeric viral particles. Summary data from 5 different donors are shown in (G). Ordinary one-way ANOVA with repeated measures was used for (C) and (G). P values were adjusted by the Holm-Sidak method. *, P<0.05; **, P<0.01; ***, P<0.001. Data are presented as mean±SD.

FIGS. 10A-10D: mACE2-CAR_sIL-15 enhances NK cell cytotoxic activity against SARS-CoV-2 spike protein expressing target cells. (A) Representative real-time cell analysis (RTCA) data showed NK cell cytotoxicity against A549-spike or parental A549 cells at an effector (E)/target (T) ratio of 1:8. The light dashed lines represent the error bars (presenting as SEM) correlated with the dark line of the same color. Each experiment was performed with cells from 4 different donors. (B) Data from the RTCA assay at different E/T ratios were summarized for cells from 4 different donors. (C) The expression of CD107a, TNF-α, and IFN-γ was measured on transduced NK cells after NK cells were co-cultured with A549-spike or parental A549 cells for 4 hat an E/T ratio of 4:1. Data were summarized for NK cells from at least 5 different donors. (D) IL-15 production by ex vivo-expanded tEGFR control NK cells or mACE2-CAR_sIL-15 NK cells cultured in the presence or absence of A549-spike cells for 72 h. Data were summarized for NK cells from 4 different donors. Two-way ANOVA was used for (B) and (C). Ordinary one-way ANOVA was used for (D). P values were adjusted by the Holm-Sidak method. ns, not significant; *, P<0.05; **, P<0.01; ***, P<0.001. Data were presented as mean±SEM for (A) and (B) and as mean±SD for (C) and (D).

FIGS. 11A-11G: Freeze-thawed mACE2-CAR_sIL-15 NK cells show effective anti-spike activity in vitro and in vivo. (A) CAR expression in mACE2-CAR_sIL-15 NK cells post-thaw, as determined by flow cytometry. Data were summarized for cells from 3 different donors. (B) Cell viability of mACE2-CAR_sIL-15 NK cells as determined at the indicated time points post-thaw using the Muse Cell Analyzer. (C) Lysis of freeze-thawed control NK or mACE2-CAR_sIL-15 NK cells that were co-cultured with ⁵¹Cr-labeled A549-spike or parental A549 cells for 4 h. Data were summarized for NK cells from 3 different donors. (D) Tumor growth in NSG mice inoculated with firefly luciferase-labeled A549-spike cells, as monitored by changes in tumor bioluminescence. Colors indicate intensity of luminescence. (E) Summary of tumor burden data from (D). (F, G) Representative images of hematoxylin and eosin (H&E) staining for lung tissues of mice after the indicated treatments. Numbers of metastatic nodules in the lungs of mice were counted, and summary data are shown in (G). A paired t test was used for (A), two-way ANOVA for (C), multiple t tests for (E) and an unpaired t test for (G). P values were adjusted by the Holm-Sidak method. ns, not significant; *, P<0.05; **, P<0.01; ***, P<0.001. Data were presented as mean±SEM for (C) and others are presented as mean±SD.

FIGS. 12A-12E: mACE2-CAR_sIL-15 NK cells protect against live SARS-CoV-2 viral infection in the K18-hACE2 humanized mouse model. (A) Schema for in vivo studies using K18-hACE2 humanized transgenic mice. (B) Relative body weights after intranasal infection with 1×10² plaque-forming units (PFU) of SARS-CoV-2. N=5 mice/group. (C) Relative body weights after intranasal infection with 1×10³ plaque-forming units (PFU) of SARS-CoV-2. N=5 mice/group. (D) Survival of mice from (C). (E) Viral RNA levels are shown for brain and lung from mice after intranasal infection with 1×10³ plaque-forming units (PFU) of SARS-CoV-2. N=5 mice/group. Two-way ANOVA was used for (B), mixed-effects analysis for (C) and a Wilcoxon test for (D). P values were adjusted by the Holm-Sidak method. *, P<0.05; **, P<0.01; ***, P<0.001; ****, P<0.0001. Data are presented as mean±SD.

FIGS. 13A-13D: Freeze-thawed mACE2-CAR_sIL-15 NK cells show effective anti-spike activity in vitro and in vivo. (A) The recovery efficiency of mACE2-CAR_IL-15 NK cells post-thaw was determined by comparing the cell numbers before and after a freeze-thaw cycle. (B) The viability of mACE2-CAR_IL-15 NK cells was determined using a Muse Cell Analyzer immediately post-thaw. (C-D) The A549-spike engrafted NSG mice were treated with PBS, tEGFR control, sIL-15 control, or mACE2-CAR_IL-15 NK cells (4 mice/group) and followed by euthanasia on day 20. Peripheral blood, spleens, and livers were harvested and analyzed for the expression of human (h)CD45 (human lymphocyte marker), hCD56 (human NK cell marker), and hLNGFR (CAR expression marker) by flow cytometry.

FIGS. 14A-14B: Depletion of endogenous immune cells in K18-hACE2 transgenic mice. (A-B) Shown is the engraftment of hLNGFR-expressing mACE2-CAR_IL-15 NK cells after lymphodepletion (A) or without lymphodepletion (B). Rows from top to bottom are PB, LV, SP, and LUNG for both (A) and (B).

FIGS. 15A-15N: Amino acid sequences of ACE2 CAR and Spike Protein scFv CAR with IgG1 spacer, CD28 transmembrane domain, CD28 co-stimulatory domain, and CD3 zeta domain. (A) wt ACE2 Long CAR (SEQ ID NO:49), without signal sequence (SEQ ID NO:57). (B) wt ACE2 Short CAR (SEQ ID NO:50), without signal sequence (SEQ ID NO:58). (C) R273S ACE2 Long CAR (SEQ ID NO:51), without signal sequence (SEQ ID NO:59). (D) R273S ACE2 Short CAR (SEQ ID NO:52), without signal sequence (SEQ ID NO:60). (E) H345F ACE2 Long CAR (SEQ ID NO:53), without signal sequence (SEQ ID NO:61). (F) H345F ACE2 Short CAR (SEQ ID NO:54), without signal sequence (SEQ ID NO:62). (G) H345S ACE2 Long CAR (SEQ ID NO:55), without signal sequence (SEQ ID NO:63). (H) H345S ACE2 Short CAR (SEQ ID NO:56), without signal sequence (SEQ ID NO:64). (I) Ab38 scFv CAR (SEQ ID NO:65). (J) Ab24 scFv CAR (SEQ ID NO:66). (K) Ab36 scFv CAR (SEQ ID NO:67). (L) Ab39 scFv CAR (SEQ ID NO:68). (M) Ab37 scFv CAR (SEQ ID NO:69). (N) Ab27 scFv CAR (SEQ ID NO:70).

FIG. 16 presents expression of ACE2 in a Normal Human Tissue Array by immunohistochemistry (IHC), where the darker stain, most remarkable in the small and large intestines, testis and kidney indicates the expression of ACE2. This figure presents the susceptibility of different organs or tissues to SARS-CoV-2 infection via assessment of ACE2 expression by immunohistochemistry (IHC) staining of a human tissue array. We observed that lungs only showed a moderate level of ACE2 expression, while expression in kidney, small and large intestines, and testis was very high, as noted above.

FIG. 17 presents immunohistochemical staining of ACE2 in human (panels A-B) and mouse (panels D-F) lungs. In human lung, ACE2 was expressed in type 2-like cells of alveoli epithelium (panel C). In mouse lung, ACE2 was expressed in a subset of bronchiolar epithelial cells and in type 2-like cells of alveoli epithelium (panel F). Arrows in panels C and F indicate ACE2 positive cells.

FIG. 18 presents immunohistochemical staining of ACE2 (receptors for the SARS-CoV-2 virus) in autopsy samples from COVID-19 patient. The human ACE2 (hACE2) expression levels of different organs or tissues of autopsy samples of COVID-19 patients by immunohistochemistry (IHC) staining. We observed that lungs only showed a moderate level of ACE2 expression, while expression in heart, kidney, and testis was very high.

FIG. 19 shows three-dimensional models of key residues involved in interaction between hACE and its substrate, angiotensin-II. Arrows indicate two key residues, Q282 and H354, involved in interaction of hACE and angiotensin-II, which were identified using PyMOL of the Schrodinger suite

FIG. 20 presents sequence alignments showing the identification of potential key residues involved in interaction between hACE2 and its substrate. The corresponding residues in hACE2, R273 and H345, were marked. The two residues that are highly conserved across mammal species are indicated by an asterisk.

FIG. 21 is a picture of an experiment showing the neutralization of SARS-CoV-2 Spike pseudotype VSV virus by hACE2. In this figure, “PD” is a positive control containing plasma from a donor following successful recovery from COVID-19 infection, and “Anti-LILRB4 antibody” is a negative control antibody. The wildtype hACE2 efficiently prevented plaques forming, which happens following viral entry onto Vero cells in a neutralizing assay using SARS-CoV-2 Spike pseudotyped VSV.

FIG. 22 is a picture of a SDS-PAGE experiment showing the expression and purification of hACE2 wild type (WT) and three mutant ACE2 proteins (R273S, H345F, and H345S) expressed in HEK293 cells and purified by protein A chromatography. The purity was estimated over 90% based on SDS-PAGE gel analysis.

FIG. 23 is a bar graph showing the inactivation of the enzymatic conversion of Angiotensin II into Angiotensin 1-7, by wild type ACE2 protein or each of three mutated ACE2 proteins (R273S, H345F, and H345S). The enzymatic activity of the wildtype and muteins were quantified by using an Angiotensin II converting enzyme (ACE2) activity assay kit. The activity of conversion of Angiotensin II into Angiotensin 1-7 by the different proteins is shown on the y-axis (Specific activity (mU/mg), and the different experiments are shown on the x-axis. In this figure, “NC”: negative control (purified leukocyte immunoglobulin like receptor B4 protein), “WT”: wild type. Compared with wildtype hACE2, enzymatic activity of three muteins was completely abolished.

FIG. 24 is a graph showing the binding affinity of wild type (WT) hACE2 and 3 mutant ACE2 proteins (R273S, H345F, and H345S) to the SARS-CoV-2 spike protein as measured by ELISA. No significant change of binding affinity was observed for all three mutants. This retention of binding to the spike protein by the three mutant hACE2 proteins may be due to the fact that the mutated residues are distal to the spike binding site.

FIG. 25 presents pictures of experiments showing the blocking of infection of pseudovirus expressing SARS-CoV2-Spike by supernatants collected from epi293 cells. The supernatants collected from lentiviral infected expi293 cells expressing the wild type (WT), R273S, H345S, or H345F mutant protein were used to block infection of Vero cells from forming plaques by SARS-CoV-2 Spike pseudotyped VSV. No treatment served as negative control and shows “too many to count” infected cells on the plate, while commercially available purified WT ACE2 protein served as positive control showing essentially <20 infected cells. The data presented on this figure show that the supernatants from both the wild type ACE2 protein and the three mutant ACE2 proteins could drastically inhibit the entrance of the spike protein into the vero cells as illustrated by a greater than 40-fold reduction in the number of infected cells when sample areas of the cultured Vero cells were assayed and compared to the negative control.

FIG. 26 is a bar graph showing exemplary results of a selection of clones producing single chain antibodies (scFv) against the spike S1 SARS-CoV-2 Receptor Binding Domain (RBD) from a fully human naïve antibody phage display library, at different rounds (Rd) in the selection process. The antigen binding activity of phage pool from each round of selection was assessed by ELISA. The screen was realized against the RBD fused to a His-tag (RBD-his, the target antigen), the S1 portion of the full-length spike protein fused with CH2 and CH3 domains of a Fc receptor (S1-Fc) as positive control, and BSA (Bovine Serum Albumin) as negative control. The result showed that antigen specific antibodies were rapidly enriched during the selection process. Moreover, the antibodies selected against RBD also bound to the spike protein, indicating that they bound to the functional conformation. BSA served as negative control.

FIG. 27A-27C present three-dimensional bar graphs showing exemplary results of the screening of unique clones using enzyme-linked immunosorbent assay (ELISA) screening of scFv candidates for binding to the SARS-CoV-2 Receptor Binding Domain (RBD) of the spike protein, from 96 single clones from the second, third, and fourth round of selections. (A) is a three-dimensional bar graph showing the results of an ELISA screen of 96 single clones selected at round 2 (Rd2) of the selection process. (B) is a three-dimensional bar graph showing the results of an ELISA screen of 96 single clones selected at round 3 (Rd3) of the selection process. (C) is a three-dimensional bar graph showing the results of an ELISA screen of 96 single clones selected at round 4 (Rd4) of the selection process. (A-C) show that the percentage of antigen positive clones dramatically increased from the second to third or fourth round of selection.

FIGS. 28A-28B present the half maximal effective concentration (EC₅₀) values of 51 unique single chain variable fragment (scFv) directed against SARS-CoV-2 receptor binding domain (RBD) of the spike protein from a scFv phage display library. (A) is a line graph showing the Optical Density at a wavelength of 450 nm (PD₄₅₀) values of antibody (ab) 3 to 53 (ab3 to ab 53) as function of the log concentration (in nM) of each antibody. (B) is a table showing the EC₅₀ values (in nanomolar, nM) of ab3 to ab53 as deduced from A. In B, underlined text indicates three antibody candidates with binding to the RBD of the spike protein at affinities greater than that of the wild type ACE2 for the RBD of the spike protein (EC₅₀=16 nM; see FIG. 29, below).

FIG. 29 is a line graph showing the affinity and EC₅₀ of the wild type ACE2 for the SARS spike S1 protein (“Antigen” values) in comparison to a BSA negative control, found to be consistent with the value reported in the literature.

FIG. 30 presents results of a SARS-CoV-2 spike pseudotyped lentivirus (VSV) assay, showing the neutralization of SARS-CoV-2 Spike pseudotyped lentivirus by scFvs (arrows identify scFv candidates with strong blocking of viral entry). As positive controls, ACE2 and serum from a convalescent patients, but not an irrelevant antibody, efficiently blocked viral entry. To screen antibodies with neutralizing potency, twenty one scFvs with the highest binding affinity were selected. From left to right, top to bottom, the anti-spike RBD scFvs tested were as follows: ab39, ab37, ab27, ab38, ab32, ab24, ab18, ab30, ab34, ab21, ab19, ab44, ab36, ab33, ab26, ab41, ab45, ab35, ab40, ab31, and ab20.

FIG. 31 presents both a bar graph and VSV assay results (graphically and experimentally) in the plaque forming assay of the neutralization of SARS-CoV-2 Spike pseudotyped VSV virus by three selected scFvs (ab38, ab24, ab36). Controls: no virus, only virus, serum from a convalescent COVID+patient, ACE2 at 1:1 dilution and 1:100 dilution, and a 1:100 dilution of an irrelevant antibody (anti-LILRB4). The controls with ACE2 present efficiently blocked viral entry. The three scFvs (ab38, ab24, ab36) shown in the bottom row of pseudovirus infectivity the plaque assay exhibited potent neutralizing activity. Quantification of cells expressing GFP plaque forming units (an indication of viral infectivity) for each condition is shown in the graph to the left

### DETAILED DESCRIPTION

Described below are studies describing the preparation of CAR targeted to SARS-CoV-2 Spike protein and NK cells expressing such CAR. Also described are studies showing that such CAR are cytotoxic to cells expressing SARS-CoV-2 spike protein.

The outbreak of coronavirus disease 2019 (COVID-19), which is caused by the novel coronavirus severe acute respiratory syndrome coronavirus-2 (SARS-CoV-2), has escalated into a pandemic (1). As of Jun. 8, 2021, more than 173 million COVID-19 cases have been reported globally and more than 3.7 million deaths (2). The public health and economic consequences have been devastating. Currently, the standard of care for COVID-19 patients includes oxygen therapy and ventilation, along with the antiviral remdesivir and the anti-inflammatory dexamethasone. Remdesivir (3, 4) and dexamethasone (5) have been approved for emergency therapeutic use for COVID-19, and each of these agents improved patient outcomes in clinical trials. However, remdesivir has limited efficacy (6) and dexamethasone is a steroid without direct anti-viral efficacy. Thus, there remains an urgent need for effective therapeutics that prevent or treat COVID-19. Cellular immunotherapies harness existing immunity to fight disease and could be beneficial against SARS-CoV-2.

Natural killer (NK) cells are innate immune lymphocytes that specialize in the recognition and rapid lysis of “abnormal cells”, including cells infected with viruses, allogeneic cells, and tumor cells without antigen pre-sensitization or human leukocyte antigen (HLA) matching (7, 8). Although NK cells are universal killers in the immune response against certain viruses or tumors, genetically modifying NK cells to express chimeric antigen receptors (CARs) can further improve NK cell targeting (9). NK cell lines, primary NK cells from peripheral blood and umbilical cord blood (UCB) and induced pluripotent stem cells have been used for CAR NK cell manufacturing (10). Like peripheral blood, UCB is a rich source of primary human NK cells and a readily available donor source with known HLA genotyping and specific NK receptor profiles; approximately 800,000 UCB units were stored in public cord blood banks, and more than 5,000,000 in private cord blood banks (11). Recently, a clinical trial that incorporated IL-15 into UCB CAR NK cells targeting the anti-CD19 antigen showed impressive outcomes in treating certain hematological malignancies (12). These results provide an encouraging foundation for the clinical use of CAR NK populations.

The surface of SARS-CoV-2 is covered by the glycosylated spike (S) protein which can bind to the host cell receptor angiotensin-converting enzyme 2 (ACE2), mediating viral cell entry (13). The S protein consists of an N-terminal subunit (S1), which mediates receptor binding, and a C-terminal subunit (S2) responsible for virus-cell membrane fusion (14). Neutralizing antibodies that target the receptor-binding domain of S1 can inhibit infection by blocking binding to ACE2 (15-19). Due to the rapid and potent nature of innate immune responses to viral infection, NK cells should be capable of fighting COVID-19; however, endogenous NK cells lack specificity against SARS-CoV-2. Since S1 binds to the ACE2 receptor, which is not naturally expressed on the NK cell surface, we speculated that engineering NK cells to express ACE2 should enhance their anti-SARS-CoV-2 activity.

These results demonstrated a promising therapeutic application of NK cells against SARS-CoV-2 infection. Because NK cells do not express the ACE2 target protein for SARS-CoV-2, we utilized UCB to generate CAR NK cells that overexpress the extracellular domain of ACE2. These engineered NK cells bind to SARS-CoV-2 spike recombinant protein and VSV-SARS-CoV-2 chimeric viral particles. Since mature NK cells have a short lifespan with poor in vivo persistence both in humans and in mice (24), we incorporated our CAR NK cells with a gene encoding soluble human IL-15 (sIL-15), a crucial cytokine for NK cells persistence (25-27). Results herein show, inter alia, that NK cells with sIL-15, but not NK cells without sIL-15, were detectable in mice at 19 days post infusion. Both in vitro and in vivo, the mACE2-CAR_sIL-15 NK cells showed striking efficacy compared to control NK cells, both in cytotoxic activity and in reducing SARS-CoV-2 viral load and SARS-CoV-2-associated death. The potential clinical benefit of this cellular therapeutic approach is supported by a recent clinical trial that utilized UCB to generate CD19 CAR NK cells along with IL-15 to produce significant anti-tumor activity against CD19-positive lymphoid malignancies (12). The results shown herein are also supported by our previous work demonstrating effective NK cell-mediated clearance of herpes simplex virus (HSV) 1 infection in vivo (28). The availability of an “off-the-shelf” CAR NK cell-based therapy for high-risk COVID-19 patients, who once infected have few treatment options, could have immediate clinical benefit. Finally, results herein demonstrate, inter alia, equivalent potency between our fresh CAR NK populations and our cryopreserved CAR NK populations suggests that in some embodiments, the mACE2-CAR_sIL-15 NK cell populations could be cryopreserved, shipped, and then stored at any medical center, allowing for immediate thawing and infusion at the first sign of clinical deterioration in any high-risk individual infected with SARS-CoV-2.

The immunopathology of SARS-CoV-2 is based on the dysfunction of both the innate and cell-mediated immune responses. Many studies have revealed that COVID-19 patients have significantly decreased numbers of NK cells and cytotoxic T cells compared to non-infected controls (29-31), and patients with severe COVID-19 have fewer of these cytolytic cells than patients with mild COVID-19 (32). Furthermore, the NK cell inhibitory receptor NKG2A (29) and T cell exhaustion markers PD-1 and Tim-3 (31) are overexpressed in COVID-19 patients as compared to healthy donors. As shown herein, when encountering virally infected cell, mACE2-CAR_sIL-15 NK cells produced robust cytokines such as IFN-γ and TNF-α. The soluble (s)-IL-15 produced by mACE2-CAR_sIL-15 NK cells maintained human CAR NK cell survival in vivo and also enhanced the anti-viral activity of endogenous immune cells such as CD8⁺ T cells and NK cells. IL-15 is shown essential for maintaining the homeostasis of T cells (33, 34). In human monocytes, IL-15 can induce the production of IL-18 and monocyte chemotactic protein 1, which attracts neutrophils and monocytes to infection sites (35). In macrophages, IL-15 also functions as a potent autocrine regulator of proinflammatory cytokine production (36). Taken together, this suggests that the addition of an immune effector cells therapy, such as mACE2-CAR_sIL-15 NK cells, corrected an immunological deficiency caused by SARS-CoV-2 infection.

Further, NK cells may offer several advantages over CAR T cells for COVID-19 patients. Although both CAR NK cell and CAR T cell therapies use engineered immune cells to recognize and kill cells expressing a specific antigen, there are important differences. CAR T cells were the first engineered cellular immunotherapies to be approved by the FDA and therefore have a longer history of clinical use. However, unlike allogeneic T cells, allogeneic NK cells do not induce graft versus host disease (37, 38), which opens the door for the broad application of allogeneic NK cellular therapies (39). Also, allogeneic CAR NK cells appear less likely than autologous CAR T cells to cause cytokine release syndrome, a potentially fatal complication due to the release of IL-6, IFN-γ, and IL-1 (12, 40). Unfortunately, COVID-19 patients display a “cytokine storm”, with increased levels of inflammatory cytokines and chemokines (TNF-α, IL-1, IL-6, IL-18, IL-8, IL-10, MCP-1), which leads to severe pulmonary tissue damage (31, 41-43). Thus, limiting CRS is especially important for COVID-19 patients.

The COVID-19 pandemic is a global health emergency, and there is no indication that it will end soon. Here, a novel, ready-to-use, and “off-the-shelf” frozen cellular population was generated to treat COVID-19. Following GMP manufacturing and FDA clearance, the mACE2-CAR_sIL-15 NK cell population could be tested in a clinical trial for high-risk patients infected with SARS-CoV-2. It could also be tested for the treatment of other coronavirus infections that penetrate the host cell using the spike protein, such as was the case for the SARS-CoV epidemic of 2003 (44).

Thus, described herein is a novel immunotherapy approach to treat SARS-CoV-2 infection with off-the-shelf mACE2-CAR_IL15 NK cells. As described herein, the human CAR NK cell populations proved efficacious in reducing morbidity and mortality in a humanized mouse model of COVID-19.

### EXAMPLES

Spike protein-targeted CARs and their use are described in the following examples, which do not limit the scope the claims.

### Materials and Methods

### Study Design

Experiments were designed to create and test a novel NK cell populations to eliminate SARS-CoV-2-infected cells, which may treat COVID-19 in humans. The UCB NK cells or those derived from CB hematopoietic stem cells were engineered to express the extracellular domain of the SARS-CoV-2 target protein ACE2, providing specificity for SARS-CoV-2 infection. The engineered NK cells were tested for the ability to target SARS-CoV-2 and eliminate SARS-CoV-2-infected cells, both in vitro and in vivo.

### Cell Lines

The A549 cell line was purchased from the American Type Culture Collection (ATCC) and cultured in RPMI with 10% heat-inactivated FBS (Sigma-Aldrich). The GP2-293 packaging cell line was purchased from Takara Bio and cultured in DMEM supplemented with 1% GlutaMax and 10% FBS. All cells were incubated at 37° C. in a 5% CO₂ humidified incubator. No further authentication of these cell lines was performed after recent purchases. Cell morphology and growth characteristics were monitored during the study and compared with published reports to ensure their authenticity. All cell lines were routinely tested for the absence of mycoplasma using the MycoAlert Mycoplasma Detection Kit from Lonza.

### Plasmid Construction and Retrovirus Production

The retroviral vector encoding tEGFR, sIL-15 and mACE2-CAR was constructed after multiple steps of PCR amplification, gel electrophoresis and extraction, enzyme digestion, ligation, transformation, and plasmid extraction.

To generate retroviral particles, the GP2-293 cells were cultured to a confluency of 70-80% and then transfected with the constructed retroviral vectors with the envelope plasmid RD114TR by using the Lipofectamine 3000 Reagent (Thermo Fisher Scientific). The culture supernatant containing the retrovirus was harvested at 48 h post-transfection and filtered.

### Generation of CAR-Modified NK Cells

UCB units were provided from StemCyte under IRB-approved protocols. All donors provided written informed consent, which followed the ethical guidelines of the Declaration of Helsinki. NK cells were isolated by using the RosetteSep™ human NK cell enrichment cocktail (Cat #15065, StemCell Technologies) and Ficoll-Paque (Cat #17144003, Cytiva). The purity of primary NK cells was confirmed with flow cytometry using anti-CD56 (Cat #IM2474U, Beckman Coulter) and anti-CD3 (Cat #130-113-134, Miltenyi Biotec) antibodies. Frozen UCB NK cells were thawed and expanded with irradiated K562 feeder cells expressing membrane-bound IL-21 and 4-1BBL (APC K562) in the presence of recombinant human IL-2 (50 IU/ml; NIH) in Stem Cell Growth Medium (SCGM) (Cat #20802-0500, CellGenix). Expanded NK cells were transduced with retrovirus at day 5 in RetroNectin (Cat #T202, Takara Bio) coated plates, which was performed according to the manufacturer's protocol. On day 8, NK cells were co-cultured with irradiated APC K562 cells for an additional 7 days prior to being harvested for in vitro analysis or frozen (Liquid Nitrogen) for in vitro and in vivo studies.

### Cytotoxicity Assays

For RTCA cytotoxicity assays, A549 or A549-spike cells were used as target cells. First, 50 ul of cell culture medium was added to each well of an E-plate. The E-plate is a standard 96-well plate with a glass bottom coated with gold microelectrodes covering approximately 75% of the well area. The E-plate was then connected to the system to check for proper electrical contacts and to obtain background impedance readings in the absence of cells. Target cells (5000 cells in 100 μl of media) were plated into the E-plate and cultured overnight in the RTCA system installed in the CO₂ incubator. mACE2-CAR_IL15 NK and control NK cells in 100 μl media were added into the E-plate and co-cultured for at least an additional 40 hours in RTCA system. The proliferation or cytotoxicity of target cells was analyzed and plotted using the RTCA software Pro every 15 minutes in a real-time manner. The cytotoxicity of each effector was calculated with the following equation:

% of cytolysis=(CI_{no effector}−CI_{effector})/CI_{no effector}×100.

For the ⁵¹Cr cytotoxicity assay, mACE2-CAR_IL15 NK and control NK cells were co-cultured with ⁵¹Cr-labeled target cells at multiple E/T ratios for 4 hours. The supernatant was harvested from each well and transferred into 96-well LumaPlate and analyzed using a Wallac MicroBeta scintillation counter (PerkinElmer).

### CD107a Degranulation and Intracellular Cytokine Production

mACE2-CAR_IL15 NK and control NK cells were cocultured with A549 or A549-spike cells at an E/T ratio of 4:1 for 4 hours in a 96-well U-bottom plate. Anti-CD107a monoclonal antibody (mAb) (Cat #563869, BD) and GolgiPlug (1:1000 dilution) (Cat #555029, BD) were added to cultures at the start of incubation. Cells were stained with anti-CD56 (Cat #IM2474U, Beckman Coulter), anti-LNGFR (Cat #557196, BD) or anti-EGFR (Cat #352904, BioLegend) mAbs and then stained intracellularly with IFN-γ (Cat #563563, BD) and TNF-α (Cat #557647, BD) mAbs. The stained cells were analyzed by flow cytometry, and the data were analyzed with FlowJo software.

### IL-15 Cytokine Secretion

Supernatants were harvested after mACE2-CAR_IL15 NK and control NK cells were co-cultured without or with A549-spike cells for 72 hours. IL-15 concentrations were measured with the human IL-15 Quantikine ELISA kit (Cat #S1500, R&D) following the manufacturer's instructions. Each experiment was performed in triplicates with repeating at least 3 times.

### Assessment of Binding of mACE2-CAR_IL15 NK Cells to a Recombinant S1 Protein Subunit

mACE2-CAR_IL15 NK and control NK cells (2×105) were incubated with 2μg of a recombinant S1 protein subunit for 2 hours at 37° C. The cells were washed twice and stained with FITC anti-His (Cat #MA1-81891, Invitrogen), APC anti-CD56, PE anti-LNGFR, or anti-EGFR mAbs for 20 minutes at room temperature. After washing, the stained cells were analyzed by flow cytometry. Data were analyzed with FlowJo software.

### Assessment of Binding of mACE2-CAR_IL15 NK Cells to VSV-SARS-CoV-2 Chimeric Viral Particles

VSV-SARS-CoV-2 chimeric viral particles were added into mACE2-CAR_IL15 NK and control NK cells (2×10⁵) in a 96-well V-bottom plate. The plate was centrifuged at 600×g for 30 minutes at 37° C., then incubated for 1 hour. The cells were washed twice and stained with an anti-S1 antibody at 37° C. for 30 minutes. The cells were washed twice and stained with an FITC goat anti-rabbit secondary antibody (Cat #554020, BD), APC anti-CD56, PE anti-LNGFR or anti-EGFR m Abs for 20 minutes at room temperature. After washing, cells were analyzed by flow cytometry, and the data were analyzed with FlowJo software.

### NSG Xenograft Model

NSG mice were purchased from the Jackson Laboratory and housed at the City of Hope Animal Facility. All experiments were approved by the City of Hope Animal Care and Use Committee. On day −1, female NSG mice (8-12 weeks old) were inoculated intravenously (i.v.) with FFLuc-labeled A549-spike cells (3.5×10⁵). On each of days 0, 2 and 4, they received an i.v. infusion (10×10⁶/mouse) of PBS, freeze-thawed tEGFR control NK cells, sIL-15 control NK cells or mACE2-CAR_sIL-15 NK cells (4 mice per group; three infusions per mouse). Tumor growth was monitored by measuring changes in tumor bioluminescence over time. Bioluminescence imaging (BLI) was performed with Lago X on days 0, 3, 6, 13, and 19. On day 20, the mice were euthanized, and tissues were harvested and prepared as FFPE blocks.

### Humanized K18-hACE2 Mouse Model

Heterozygous K18-hACE c57BL/6J mice (2B6.Cg-Tg(K18-ACE2)2Prlmn/J) were obtained from The Jackson Laboratory. K18-hACE2 transgenic mice (6-8 weeks old) received 200 μg each of anti-NK1.1 mAb (Cat #BE0036, BioCell), anti-mCD4 (Cat #BE0003-1, BioCell), and anti-mCD8a (Cat #BP0061, BioCell) plus 100 μl clodronate liposomes (Cat #CLD-8909, Encapsula NanoSciences) for macrophage depletion via intraperitoneal (i.p.) injection on day −2. Two days later (day 0), the mice were i.n. infected with SARS-CoV-2. On day 1, the mice received i.v. administration of PBS, 15×10⁶ tEGFR control NK cells or mACE2-CAR_IL-NK cells. Body weight was monitored daily after infection and calculated as the percentage of the initial body weight. Work with SARS-CoV-2 was performed in a biosafety level 3 laboratory by personnel equipped with powered air-purifying respirators.

### Viral RNA Copy Number Detection

The details were shown in our previous study (21). Briefly, the viral RNA was isolated from the homogenized tissues using the PureLink RNA Mini kit (Invitrogen). A one-step RT-PCR kit (BioRad) was used to detect the viral RNA using Applied Biosystems QuantStudio 12K Flex Real-Time PCR System. The primer sequences were CoV2-S_19F, 5′-GCTGAACATGT-CAACAACTC-3′, and CoV2-S_143R, 5′-GCAATGATGGATTGACTAGC-3′. The standard samples were serial 10-fold dilutions of a known copy number of the HKU1 virus. The results were normalized and expressed as genome equivalent copies per gram of tissue.

### Statistical Analysis

Statistical significance was assigned when P values were 0.05 or less using Prism version 9 (GraphPad). Two independent or paired groups were compared by Student's two-tailed t-tests or paired t-tests. Multiple groups were compared using one-way or two-way ANOVA or multiple t-test. P values were adjusted with the Holm-Sidak method.

### Direct Binding ELISA—ACE2 Proteins and Variants

To determine the binding affinity of either antibody-displaying phage or purified antibody against the spike protein, the purified 6× his-tagged spike protein was coated on an ELISA plate for overnight. Next day, plate was blocked with BSA for 1 hr. After blocking, samples were added and incubated for 1 hr. Bound phage or antibody was detected by using HRP-conjugated anti-HA antibody (Sino Biologics, Cat. #: 100028-MM10-H).

### Neutralizing Assay—ACE2 Proteins and Variants

To assess neutralizing activity, antibody or hACE2 was mixed with SARS-COV-2 Spike pseudotyped VSV virus and incubate for 1 hr at 37° C. After incubation, the mixture solution was transferred to Vero E6 cells seeded in a 96-well plate. After 1 hr infection, the mixture solution was replaced with fresh RPMI-1640 supplied with 10% FBS. GFP expression, which indicates pseudoviral infection, was visualized under microscope after 12-24 hr culture.

### Protein Expression and Purification—ACE2 Proteins and Variants

For expression and purification of hACE2, the HEK293T cells were transfected with a hACE2-expressing plasmid and cultured at 37° C. with 5% CO2 for 48 to 72 hr. The supernatant containing the hACE2 protein was harvested and incubated with protein A resin for 1 hr. After incubation, the resin was washed and protein was eluted by 0.2 mM citric acid (pH 2.5). Eluted protein was immediately neutralized with 1 M Tris buffer (pH 11). Concentration of protein was determined by Nanodrop. The same approaches were used for mutated hACE2 proteins.

### Angiotensin II Converting Enzyme (ACE2) Activity Assay

Angiotensin II converting enzyme (ACE2) activity assay was performed according to manufacturer's instruction (Biovision, Cat. #: K897). Briefly, purified hACE2 was mixed with substrate. Read fluorescence (excitation/emission=320/420 nm) every 15 min from 0 to 2 hr in a kinetic mode. Two time points in the linear range of the plot were chosen to calculate enzymatic activity of ACE2 or its mutants.

### Antibody Selection

Antibody selection was conducted by using solid phase panning strategy. Briefly, a human naïve antibody phage display library containing 1×10¹¹ unique clones was incubated with immobilized SARS-COV-2 Spike RBD (Sino Biological©, Cat. #: 40592-V08H) on an ELISA plate. Unbound phage was washed and bound phage was eluted with 0.1 HCl. After neutralization, eluted phage was propagated in E. coli TG-1 host cells and subjected to next round of selection. To screen antigen specific clones, single clones from output of selection were picked. Antigen binding was assessed by using direct binding ELISA as described below.

### Direct Binding ELISA—Anti-Spike scFv

To determine the binding affinity of either antibody-displaying phage or purified antibody against the spike protein, the purified 6× His-tagged spike protein, in which the spike protein was fused to six amino acids of His (e.g., encoded by CAT CAC CAT CAC CAT CAC; SEQ ID NO:A109), was coated on an ELISA plate for overnight. The next day, plate was blocked with BSA for 1 hr. After blocking, samples were added and incubated for 1 hr. Bound phage or antibody was detected by using HRP-conjugated anti-HA antibody (Sino Biological©, Cat. #: 100028-MM10-H).

### Neutralizing Assay—Anti-Spike scFv

To assess neutralizing activity of the antibodies, an antibody or a commercially available hACE2 (Sino Biological©) was mixed with SARS-COV-2 Spike pseudotyped VSV virus and incubate for 1 hour at 37° C. After incubation, the mixture solution was transferred to Vero E6 cells seeded in a 96-well plate. After 1 hour infection, the mixture solution was replaced with fresh RPMI-1640 supplied with 10% FBS. Green Fluorescent Protein (GFP) expression, which indicates pseudoviral infection, was visualized under microscope after 12-24 hour culture.

### Protein Expression and Purification—Aanti-Spike scFv

For expression and purification of scFv, E. coli cells transformed with an expression plasmid were grown until the optical density at a wavelength of 600 nm (OD₆₀₀) of the cultures reached a value of 0.6. Protein expression was induced by 0.1 mM isopropyl β-d-1-thiogalactopyranoside (IPTG) overnight. After induction, bacteria were lysed by sonication. The cell debris was removed by centrifugation. The supernatant containing scFv was then incubated with nickel resin for 1 hr. After that, the scFv bound resin was washed and protein was eluted by 200 mM imidazole. Concentration of protein was determined by spectrophotometry (Nanodrop™)

### Example 1: CAR Targeted to SARS-CoV-2

Four CAR targeted to SARS-CoV-2 spike protein were created. All included a targeting domain, a spacer, a CD28 transmembrane domain, a CD28 co-stimulatory domain, and a CD3zeta domain. The sequence encoding the CAR was followed by a sequence encoding a T2A skip sequence, a sequence encoding a soluble form of IL-15, a sequence encoding a second T2A skip sequence and a sequence encoding LNGFR. Two of the CAR, SPIKE L1H1 and SPIKE H1L1, included an ScFv targeted to Spike protein (SEQ ID NO: ______ and SEQ ID NO: ______, respectively). The other two CAR, ACE2-L and ACE2-S, include a portion of extracellular domain of ACE2 (SEQ ID NO: 29 and SEQ ID NO: 30, respectively).

### Example 2: Engineering and Expansion of NK Cells

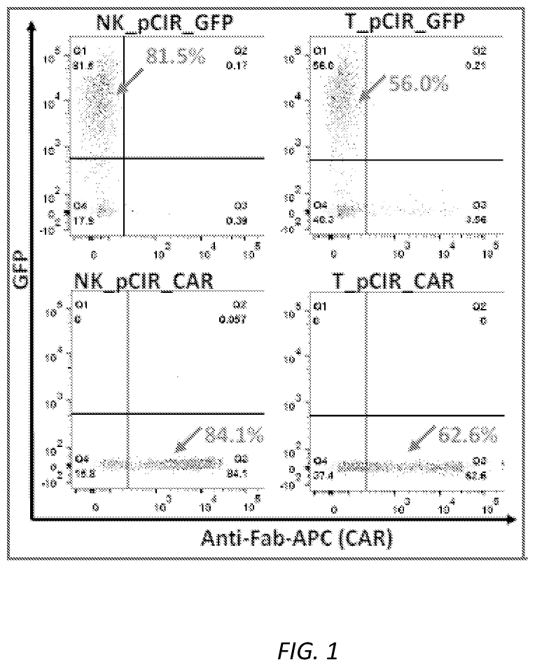

To optimize transduction efficiency, we used an efficient retrovirus-based transduction system that in turn optimizes the manufacturing of CAR NK cells. We expanded primary immature or precursor-like umbilical cord blood NK cells on a K562 feeder cell layer expressing membrane-bound IL-21 and 4-1BBL, followed by infection with a replication-defective retroviral vector, pCIR. This provides NK cells with a higher transduction efficiency and improved survival and persistence. Using this retrovirus-based system we achieved approximately 70-80% transduction of human NK cells (FIG. 1, left panels), which is comparable or better than transduction of T cells with the same retrovirus expressing a CAR (FIG. 1, right panels).

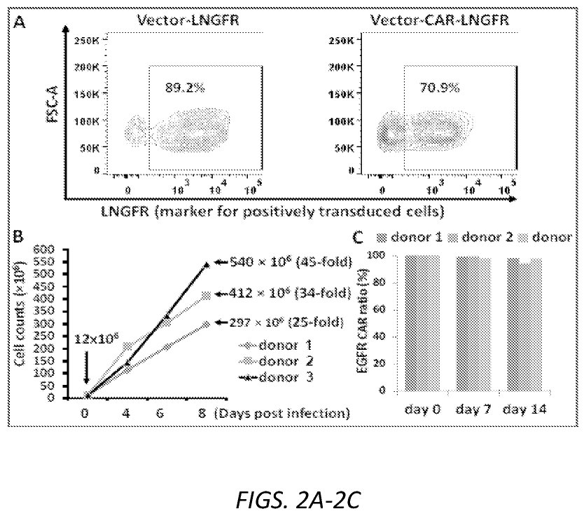

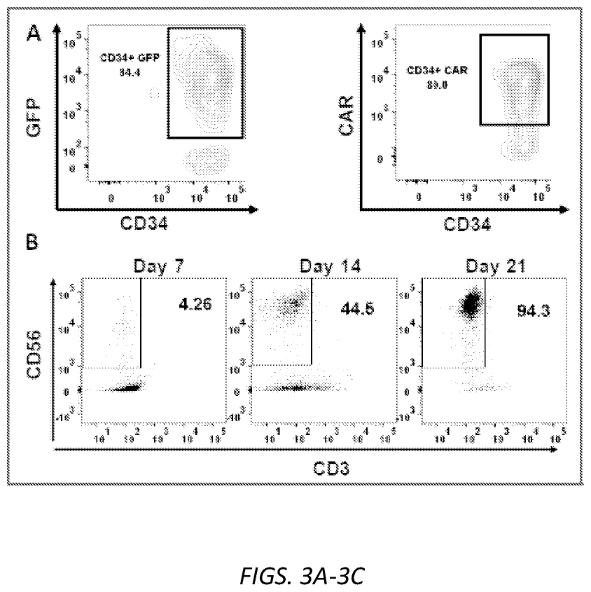

To assess the number of CAR NK cells that can be produced from one cord blood batch, we started with 1.2×10⁷ expanded CAR NK cells derived from immature and precursor-like umbilical cord blood NK cells, using K562 feeder cells that express membrane-bound IL-21 and 4-1BBL for each of the three healthy donors. We achieved —70% transduction efficiency in this experiment (FIG. 2A). We then further expanded the transduced NK cells using the K562 feeder cells. Eight days later, we were able to harvest 2.97-5.4×10⁸ CAR NK cells. Although we stopped the experiment on day 8 (FIG. 2B), the engineered NK cells continued to expand. We sorted the expanded transduced primary CAR NK cells to >98% purity and cultured them for 2-3 weeks. We observed that CAR expression remained (FIG. 2C and data not shown). We estimated that in less than two weeks of culture, it is possible we created 1.5×10⁹ CAR NK cells.

### Example 3: Distribution of Infused CAR NK Cells

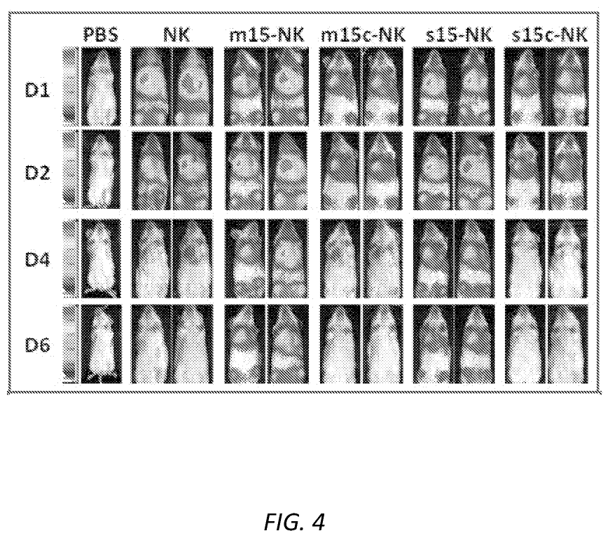

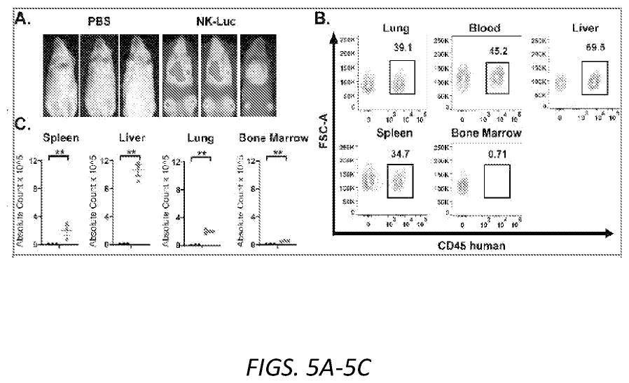

To investigate the biodistribution of engineered human NK cells after infusion, we transduced human NK with a retroviral vector expressing a luciferase gene and infused the cells into NSG mice and examined biodistribution by bioluminescence imaging (FIGS. 4, 5A-5C). Anti-human CD45 staining followed by flow cytometric analysis was used to determine and count human NK cells in different organs or tissues (FIG. 5B, 5C). This data shows that NK cells mainly traffic into the liver, lungs, and spleen. Of note, total body weight and weight of certain organs, e.g., lungs had no loss, suggesting that CAR NK cells should be safe.

### Example 4: NK Cells are Diminished in COVID-19 Patients

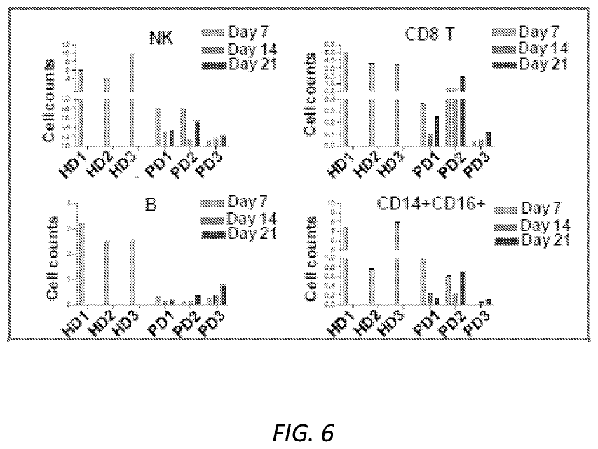

We analyzed the immune response of 12 COVID-19 patients. Preliminary analysis of the data shows that NK cell numbers are dramatically decreased, while NK cells percentages are moderately decreased. Both B cells and CD4 T cells were decreased, while CD8 cells and myeloid cells were not (FIG. 6 and data not sown).

### Example 5: Targeted CAR NK Cells Kill Spike Expressing Cells

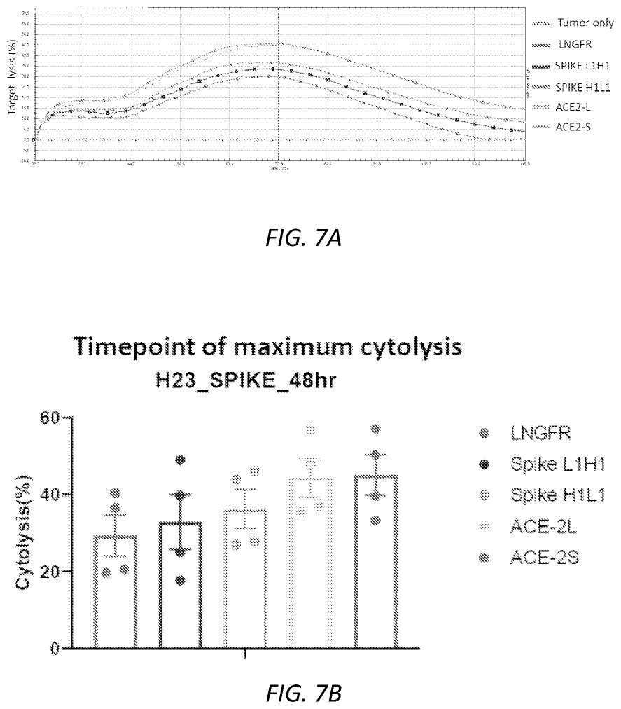

The NCI-H23 lung cell line was transfected with a lentiviral vector expressing SARS-COV-2 Spike protein. These cells were co-cultured with CAR NK cells expressing IL-15 and SPIKE L1H1 (an antibody against Spike; light chain followed by heavy chain), SPIKE H1L1 (an antibody against Spike; heavy chain followed by light chain), ACE2-L (ACE2 extracellular domain long form) or ACE2-S (ACE2 extracellular domain short form), described in Example 1. As can be seen in FIG. 4 and FIGS. 7A-7B, all four CAR constructs increased the ability of the NK cells to lyse the Spike protein-expressing cells when compared to tumor cells alone or LNGFR-transduced control NK cells.

### Example 6: Generation of mACE2-CAR_sIL-15 NK Cells

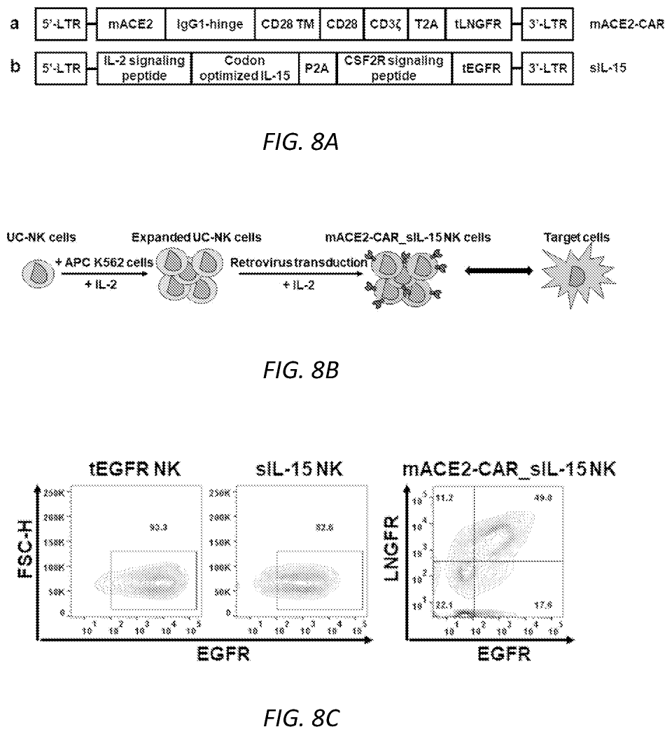

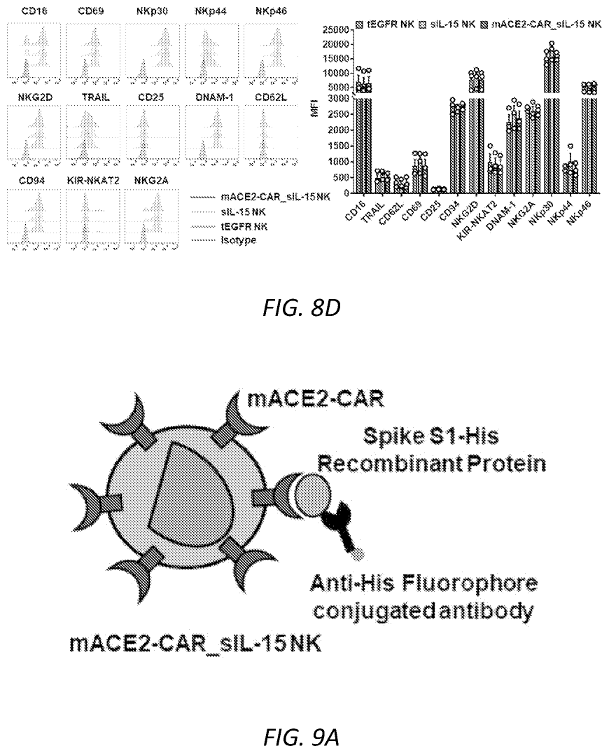

We first created a CAR NK cell-based immunotherapy to treat SARS-CoV-2 infection. We combined a portion of the SARS-CoV-2 target protein ACE2 (mACE2), which was lacking a second extracellular domain, to a CD28 and CD3ζ intracellular signaling domain to create mACE2-CAR (FIG. 8A). NK cells were isolated from UCB and cultured with antigen-presenting K562 feeder cells expressing 4-1BBL and membrane-bound (mb) IL-21 (APC K562) for 5 days. The expanding and activated NK cells were then transduced with a mixture of two retroviral vectors, one expressing mACE2-CAR and truncated (t) low-affinity nerve growth factor receptor (LNGFR) (FIG. 8A(a)) and another one expressing sIL-15 and truncated (t) epidermal growth factor receptor (EGFR) (FIG. 8A(b)) to generate mACE2-CAR_sIL-15 NK cells (FIG. 8A). Following transduction, the UCB NK cells were cultured for an additional 12 days (FIG. 8B). The transduction efficiency of control tEGFR NK cells or control sIL-15 NK cells was evaluated by measuring tEGFR expression, while CAR expression on mACE2-CAR_sIL-15 NK cells by measuring tLNGFR expression and sIL-15 expression on mACE2-CAR_sIL-15 NK cells by measuring tEGFR expression (FIG. 8C). The cells with expression of LNGFR were saved as the CAR-negative fraction to be used as a negative control in the study. To investigate the potential of IL-15 to activate the NK cells, we comprehensively characterized the mACE2-CAR_sIL-15 NK (SEQ ID NO:49) cell phenotype following is activation and expansion as shown in FIG. 8B, including expression of activating and inhibitory receptors on expanded NK populations by flow cytometry. Our data show that mACE2-CAR_sIL-15 NK cells exhibit a phenotype similar to that of control NK cells transduced with only tEGFR or NK cells transduced with sIL-15, but activated and expanded in an identical fashion as mACE2-CAR_sIL-15 NK cells (FIG. 8D).

### Example 7: mACE2-CAR_sIL-15 NK Cells Bind to SARS-CoV-2 Spike Protein and VSV-SARS-CoV-2 Chimeric Viral Particles

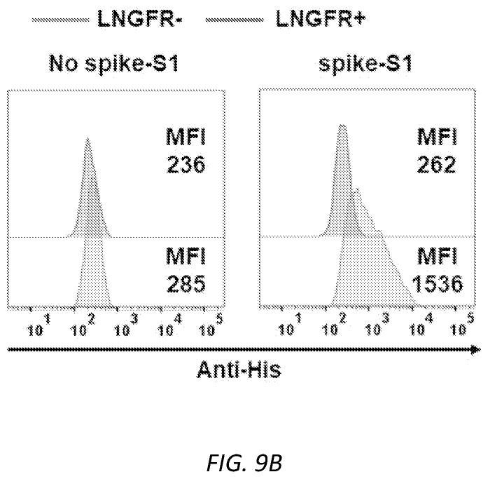

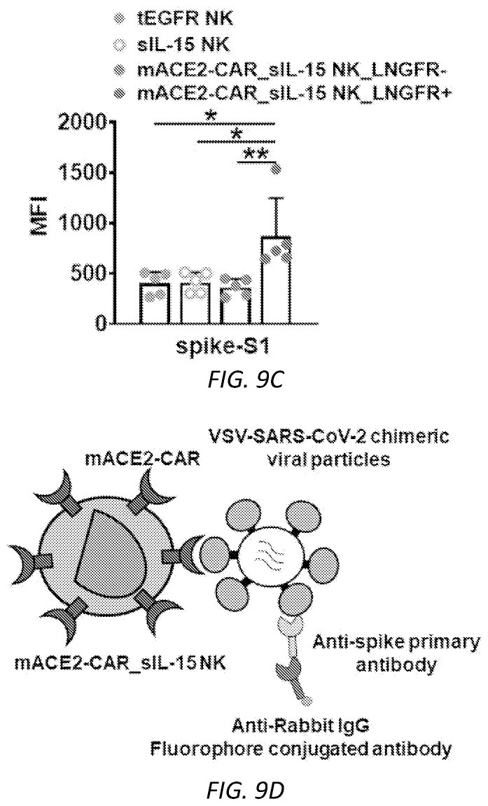

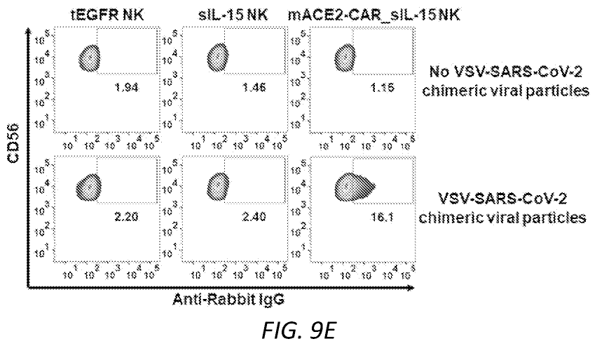

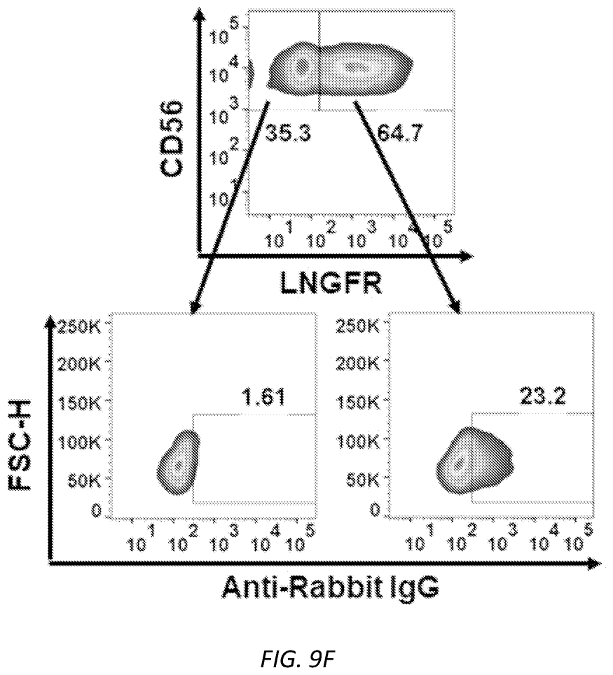

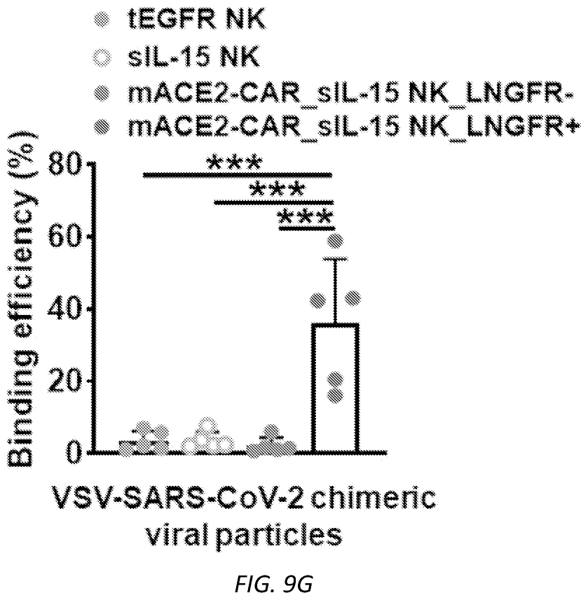

After establishing the mACE2-CAR_sIL-15 NK cells, we confirmed their ability to bind the SARS-CoV-2 spike protein. We incubated mACE2-CAR_sIL-15 NK and control NK cells with a recombinant His-tagged S1, then assessed complex formation by flow cytometry using an anti-His-conjugated antibody (FIG. 9A). Our results showed that the SARS-CoV-2 S1 protein subunit bound to mACE2-CAR_sIL-15 NK cells, but not to either control NK cells transduced with tEGFR or with sIL-15 (FIGS. 9B and 9C). Because the SARS-CoV-2 S1 protein subunit alone may not fully reflect the complexity of SARS-CoV-2 viral particles, we next tested whether mACE2-CAR_sIL-15 NK cells could bind to viral particles expressing the SARS-CoV-2 spike protein. For this purpose, we incubated mACE2-CAR_sIL-15 NK and control NK cells with chimeric vesicular stomatitis virus (VSV) particles expressing the SARS-CoV-2 spike protein (VSV-SARS-CoV-2) (20), then assessed complex formation by flow cytometry using an anti-spike protein antibody and its corresponding fluor-conjugated secondary antibody (FIG. 9D). As expected, mACE2-CAR_sIL-15 NK cells bound to the VSV-SARS-CoV-2 viral particles, whereas neither control NK cells transduced with tEGFR or with sIL-15, nor the CAR-negative fraction of mACE2-CAR_sIL-15 NK cells, bound to the viral particles (FIG. 9E-9G). We therefore concluded that mACE2-CAR_sIL-15 NK cells can specifically and strongly bind to the SARS-CoV-2 S1 protein subunit and to viral particles expressing the SARS-CoV-2 spike protein.

### Example 8: mACE2-CAR_sIL-15 Enhances NK Cell Cytotoxic Activity Against SARS-CoV-2 Spike Protein Expressing Target Cells

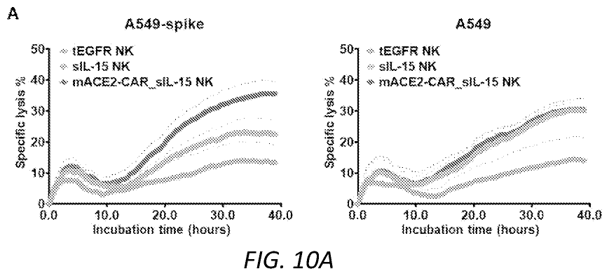

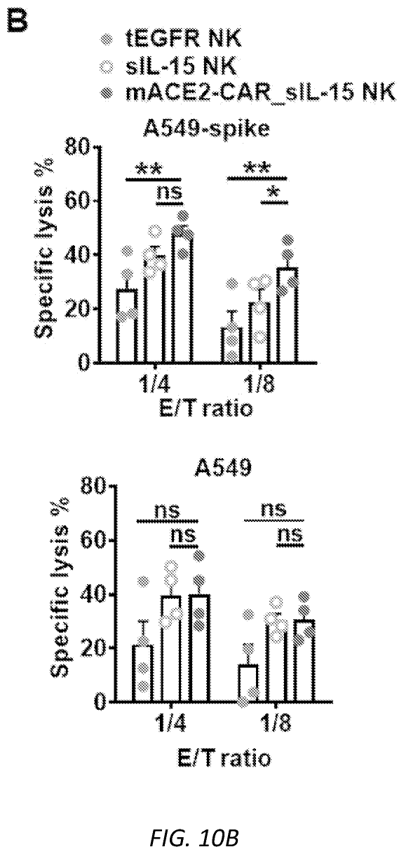

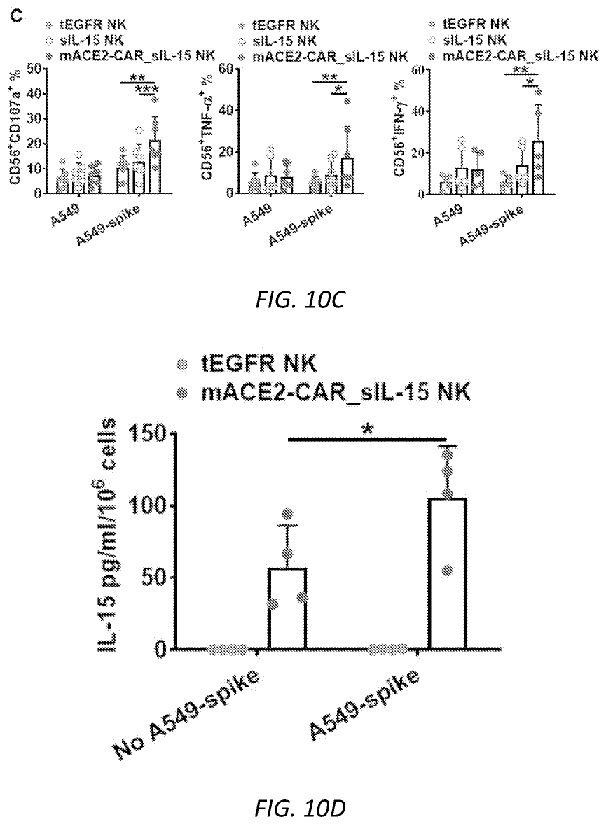

To determine whether the effector function of mACE2-CAR_sIL-15 NK cells is enhanced by interacting with SARS-CoV-2 spike-expressing target cells, we first expressed the SARS-CoV-2 spike protein in the A549 human lung carcinoma cell line (A549-spike). We incubated mACE2-CAR_sIL-15 NK and control NK cells with A549-spike or A549 cells and assessed their ability to eradicate the A549 cells using real-time cell analysis (RTCA). As shown in FIG. 10A, mACE2-CAR_sIL-15 NK cells exhibited greater cytotoxic activity against A549-spike cells in long-term cytotoxicity assays than control NK cells. We also summarized the cytotoxic activity of mACE2-CAR_sIL-15 NK cells against A549-spike and parental cells at different effector (E)/target (T) ratios, which showed increased cytotoxic activity of mACE2-CAR_sIL-15 NK cells against A549-spike cells compared to control NK cells (FIG. 10B). Next, we measured the levels of NK cell degranulation, as well as TNF-α and IFN-γ production, as parameters of NK cell function. Consistent with cytotoxicity results, mACE2-CAR_sIL-15 NK cells exhibited significantly increased release of CD107a-containing cytotoxic granules, and increased TNF-α and IFN-γ production in the presence of A549-spike cells compared to tEGFR or sIL-15 control NK cells, whereas there was no difference between the mACE2-CAR_sIL-15 NK and control NK cells in the presence of A549 cells (FIG. 10C). We measured IL-15 release after 72-hour culture of mACE2-CAR_sIL-15 NK cells and control tEGFR NK cells in the presence or absence of A549-spike cells. IL-15 was undetectable in supernatants collected from tEGFR control NK cells, however, the mACE2-CAR_sIL-15 NK cells produced small amounts of IL-15 (mean 57.2 pg/ml/10⁶ cells, range 31.6-94.3 pg/ml/10⁶ cells) in the absence of A549-spike cells, and significantly more IL-15 (mean 105.6 pg/ml/10⁶ cells, range 54.9-135.4 pg/ml/10⁶ cells) when cultured with A549-spike cells (FIG. 10D). Together, these results indicate that mACE2-CAR_sIL-15 NK cells exhibit specific and robust NK cell effector function in the presence of target cells expressing the SARS-CoV-2 spike protein, which mimic cells infected with SARS-CoV-2.

### Example 9: Freeze-Thawed mACE2-CAR_sIL-15 NK Cells Show Effective Anti-Spike Activity In Vitro and In Vivo

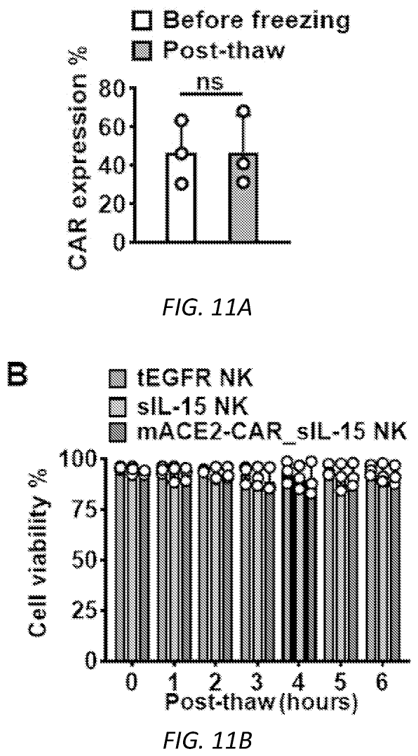

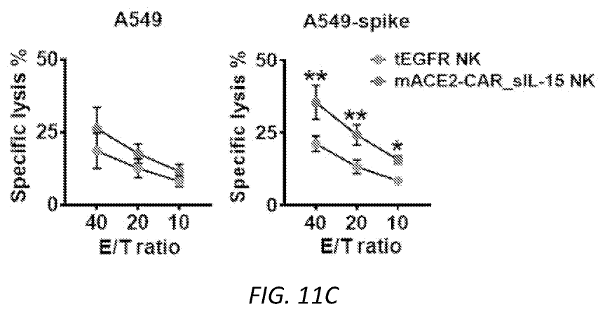

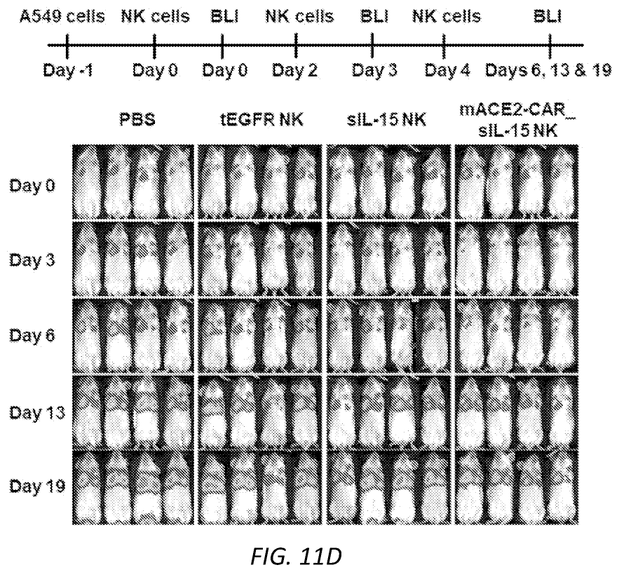

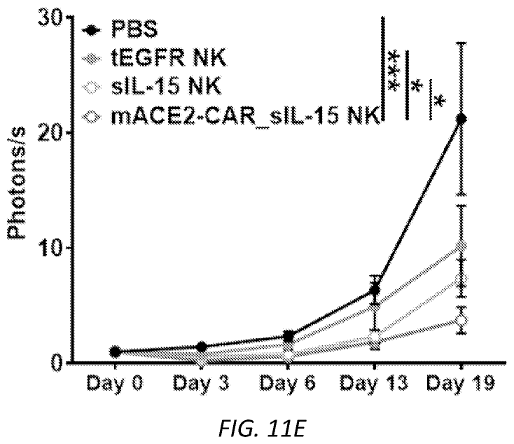

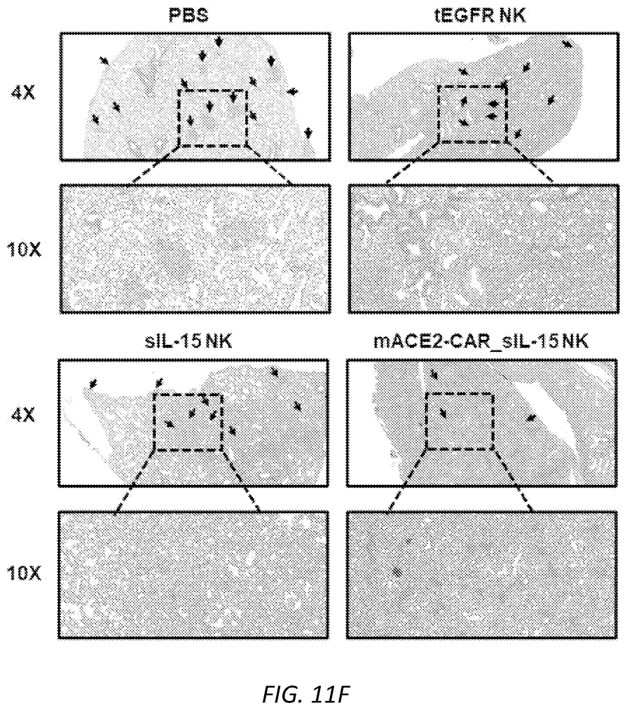

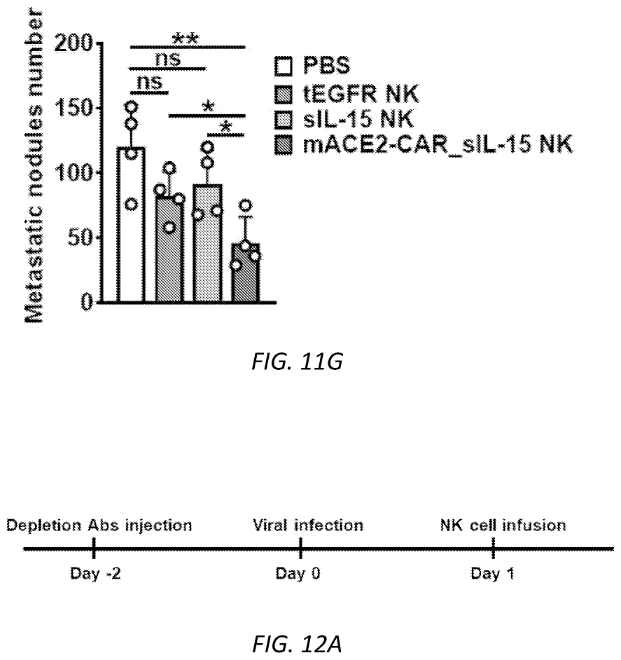

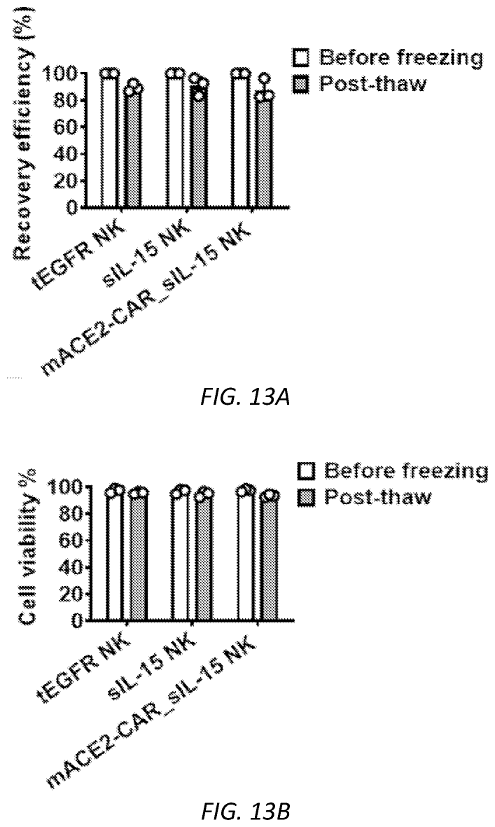

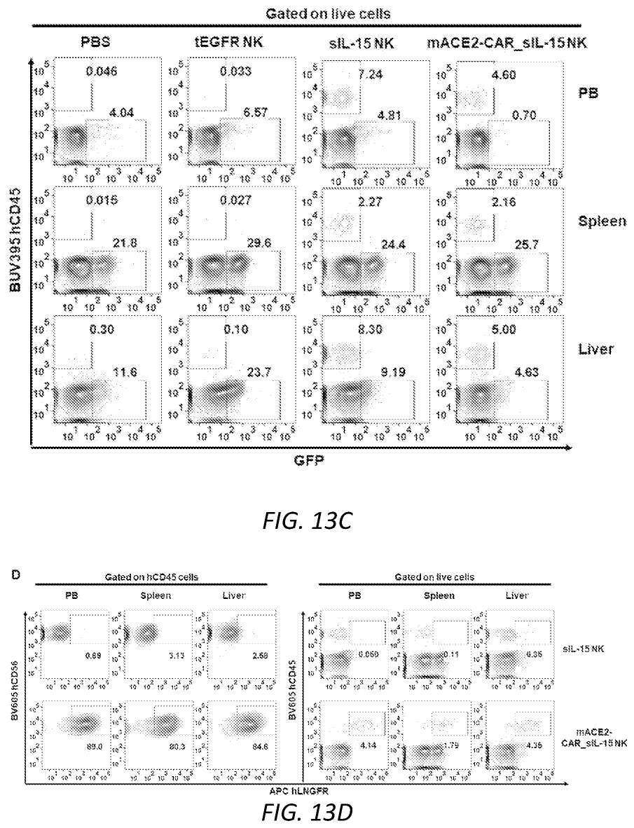

To understand the potential clinical applicability of the mACE2-CAR_sIL-15 NK cells, we evaluated the stability of the cells after cryopreservation. We first assessed their function in vitro after a freeze-thaw cycle. The efficiency of recovery of mACE2-CAR_sIL-15 NK cells after a freeze-thaw cycle was over 80% (FIG. 13A), and the cell viability was over 90% (FIG. 13B). Moreover, mACE2-CAR expression in the NK cells post-thaw was similar to that of fresh mACE2-CAR_sIL-15 NK cells (FIG. 11A). mACE2-CAR_sIL-15 NK cell viability persisted at >80%, even after 6 hours post-thaw (FIG. 11B). Next, we used the 4-hour ⁵¹Cr release assay to evaluate potency of the mACE2-CAR_sIL-15 NK cells following cryopreservation. The thawed mACE2-CAR_sIL-15 NK cells exhibited greater cytotoxic activity against A549-spike cells compared to thawed control tEGFR NK cells (FIG. 11C).

We used a firefly luciferase (FFLuc)-labeled A549-spike xenograft NOD-SCID-IL2Rγ^{-/-} (NSG) mouse model to study the in vivo anti-spike activity of mACE2-CAR_sIL-15 NK cells. NSG mice (8-12 weeks old) were inoculated intravenously (i.v.) with FFLuc-labeled A549-spike cells on day −1. On each of days 0, 2 and 4, the mice received an i.v. infusion (10×10⁶/mouse) of PBS, freeze-thawed control tEGFR NK cells, control sIL-15 NK cells or mACE2-CAR_sIL-15 NK cells (4 mice per group; three infusions per mouse). Tumor growth was monitored by measuring changes in tumor bioluminescence over time (FIG. 11D). Infusion of mACE2-CAR_sIL-15 NK cells led to improved tumor control compared to control tEGFR or sIL-15 NK cells (FIG. 11E). On day 20, the mice were euthanized, and tissues were harvested and prepared as formalin-fixed paraffin-embedded (FFPE) specimens. Using flow cytometry, we identified CAR-expressing NK cells in blood, spleen, and liver (FIGS. 13C and 13D), indicating proliferation and successful homing of mACE2-CAR_sIL-15 NK cells. Moreover, the presence of A549-spike cells in the lung was confirmed with H&E staining (FIG. 11F), and the number of lung metastases was significantly lower in mice treated with mACE2-CAR_sIL-15 NK cells compared to those treated with control NK cells (FIG. 11G). Taken together, our in vitro and in vivo data suggest that mACE2-CAR_sIL-15 NK cells retain activity after freezing, indicating that mACE2-CAR_sIL-15 NK cells may have broad applicability for treating SARS-CoV-2 infection.

### Example 10: mACE2-CAR_sIL-15 NK Cells Protect Against Live SARS-CoV-2 Viral Infection in the K18-hACE2 Humanized Mouse Model

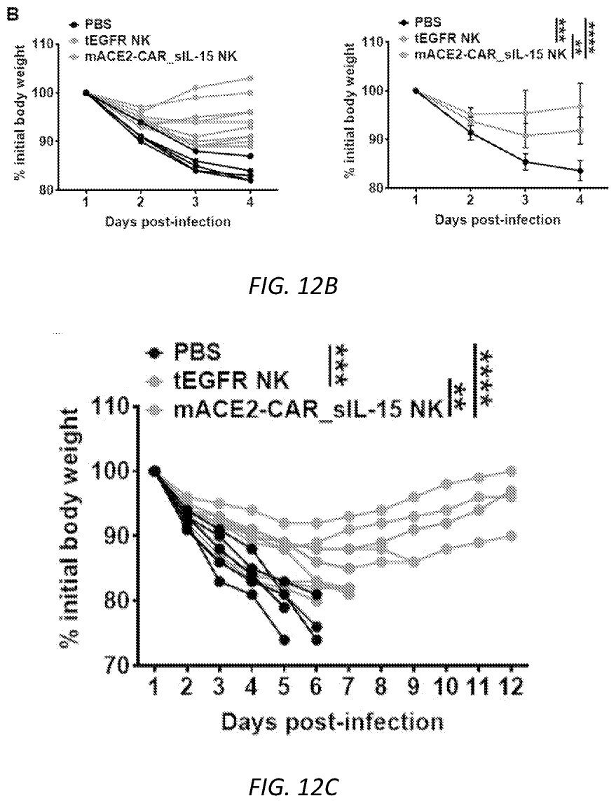

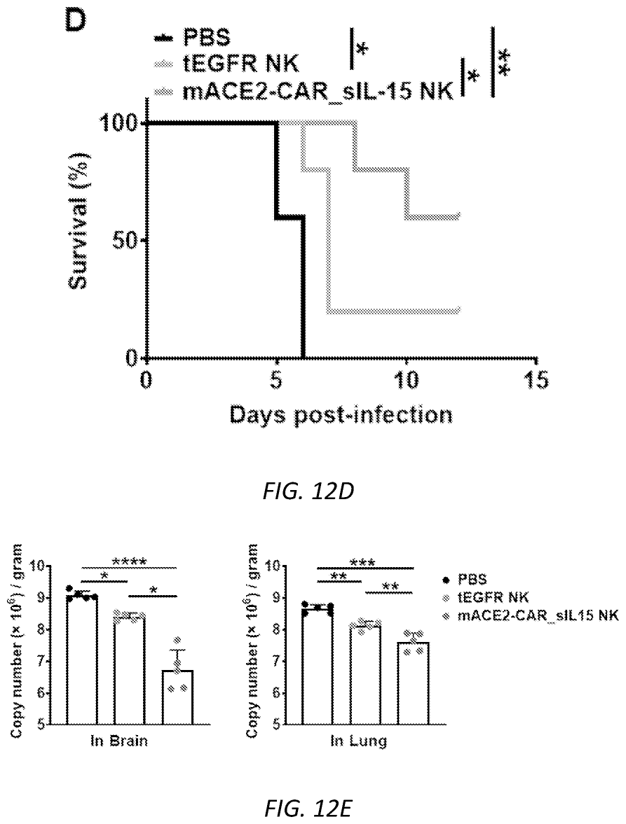

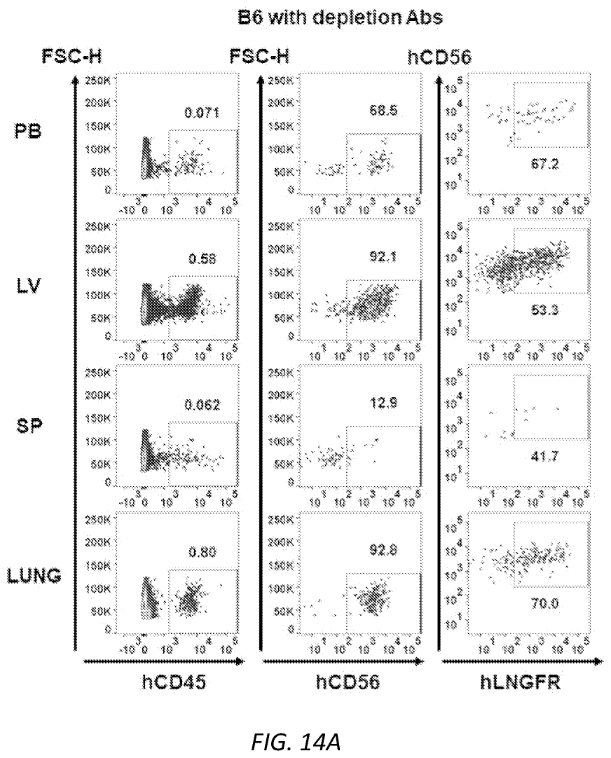

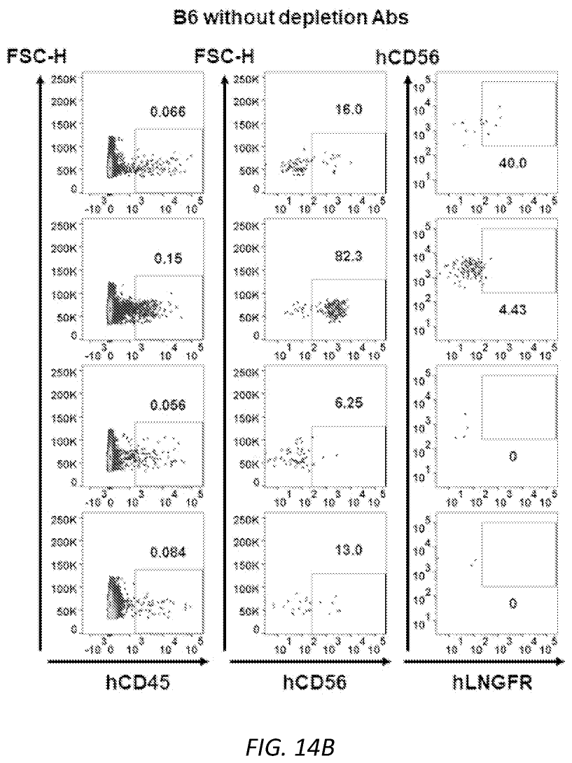

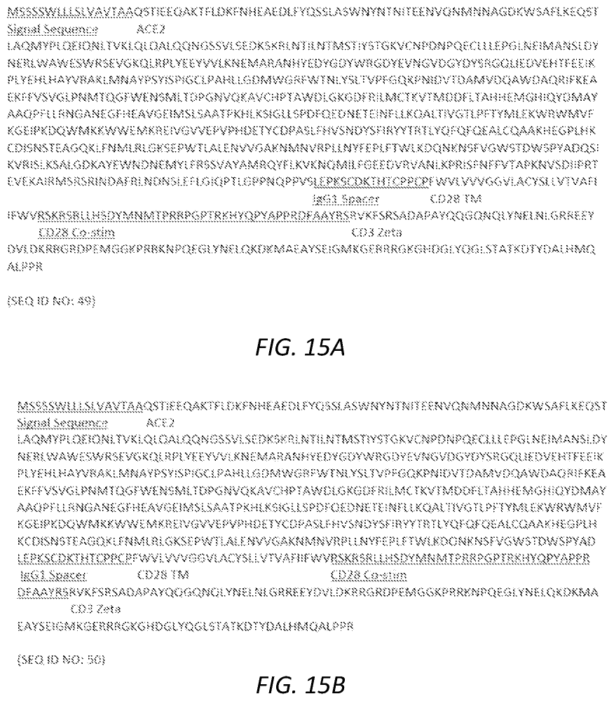

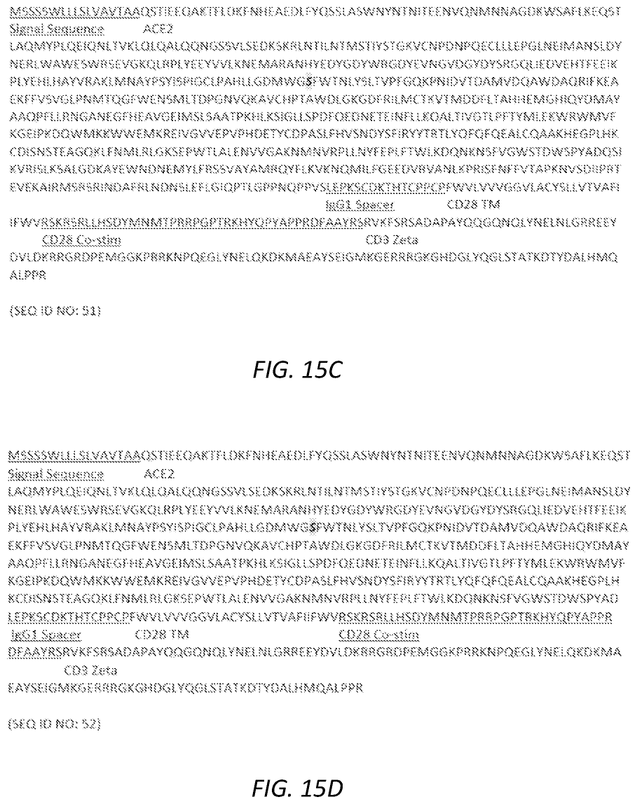

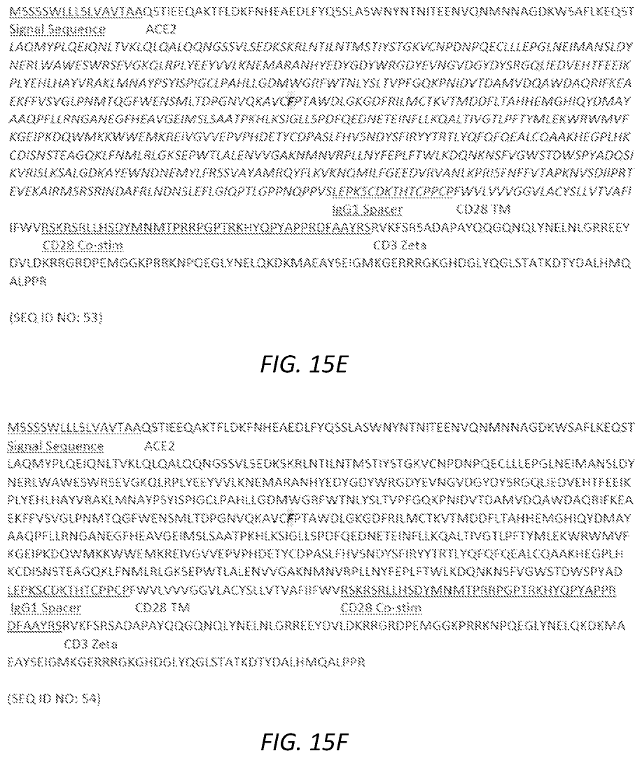

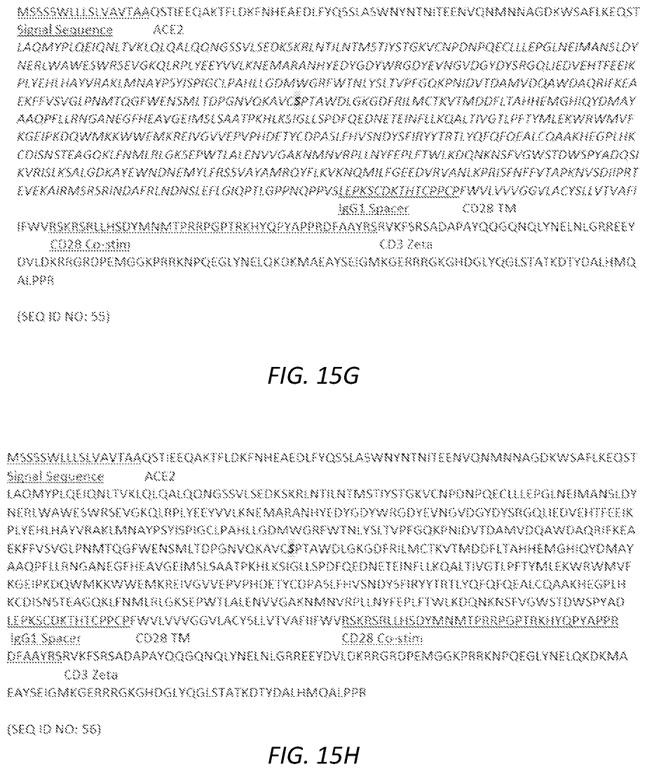

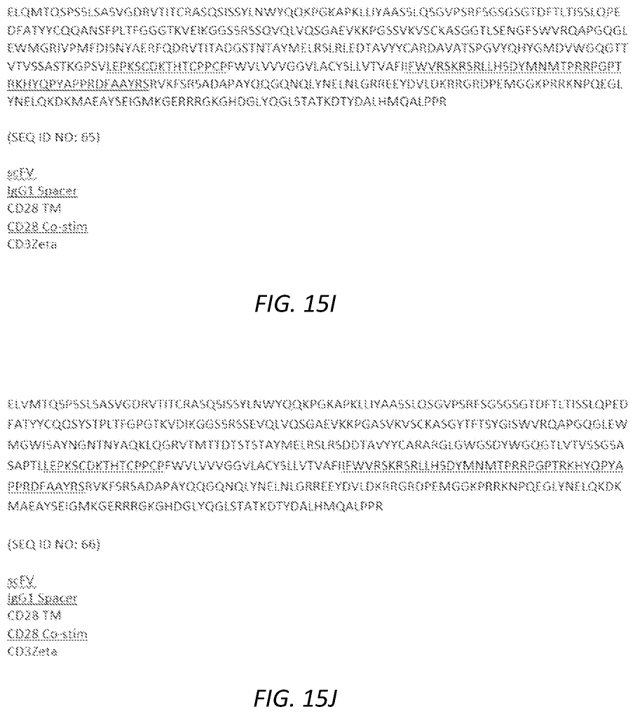

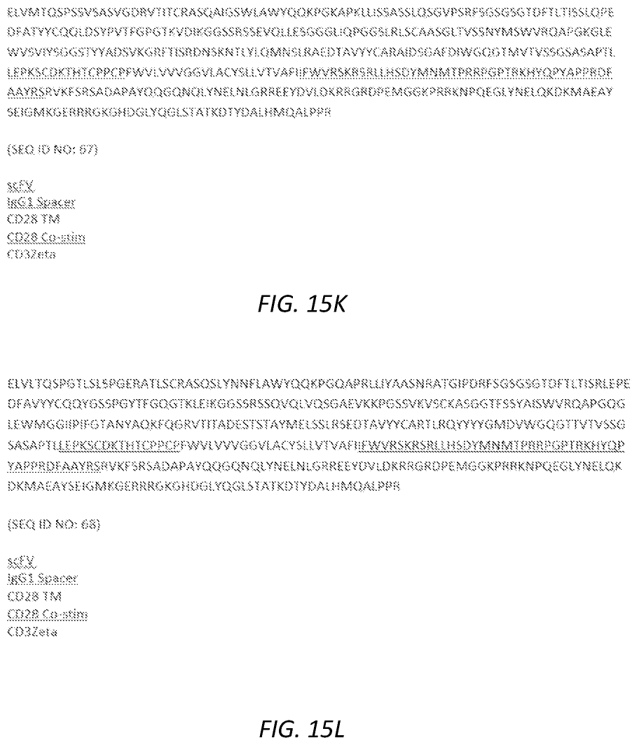

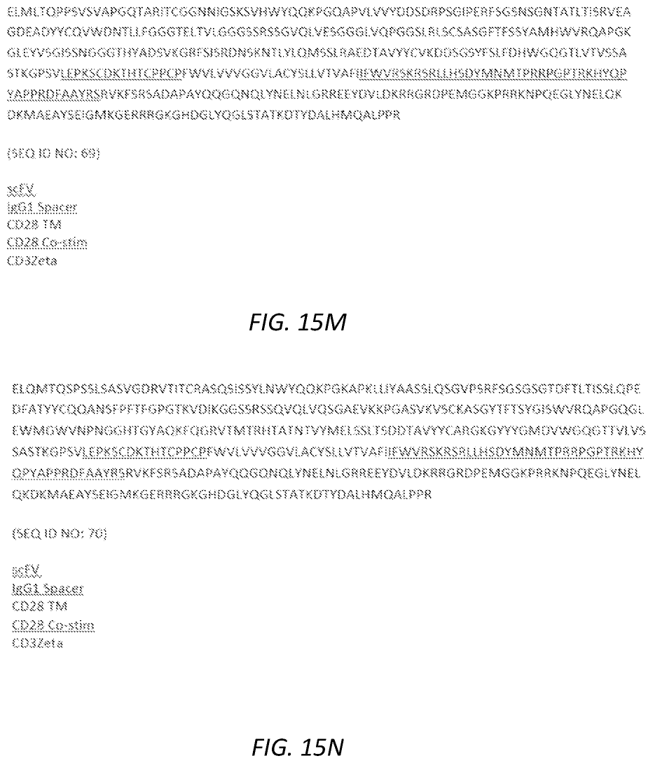

To test the ability of mACE2-CAR_sIL-15 NK cells to prevent live SARS-CoV-2 viral infection in vivo, we used K18-hACE2 transgenic mice, which express human ACE in epithelial airway cells (21). K18-hACE2 transgenic mice were depleted of endogenous immune cells to avoid potential rejection of human CAR cells day −2 before live SARS-CoV-2 infection on day 0 (FIG. 14A-14B). On day 1, mice were treated with saline, control tEGFR NK cells or mACE2-CAR_sIL-15 NK cells (FIG. 12A). The saline-treated K18-hACE2 transgenic mice begin to lose weight on days 2-4 and die of the inflammatory response on days 6-9 after intranasal (i.n.) infection with live SARS-CoV-2 (22, 23). This response was consistent with our recent characterization of K18-hACE2 mice after live SARS-CoV-2 infection (21). Body weight was monitored daily after infection and calculated as a percentage of the initial body weight. When mice were infected with 1×10² plaque-forming units (PFU) of live SARS-CoV-2, the control tEGFR NK cells could protect mice from the infection, as their body weights dropped significantly less than the body weights measured in the saline-treated group of infected mice (FIG. 12B). However, mice that received mACE2-CAR_sIL-15 NK cells after live SARS-CoV-2 infection had significantly better maintenance of body weight than mice that received control tEGFR NK cells (FIG. 12B). When mice were infected with a higher dose (1×10³ PFU) of live SARS-CoV-2, mice receiving mACE2-CAR_sIL-15 NK cells again had the best protection from SARS-CoV-2 infection as measured by maintenance of body weight (FIG. 5C). All of the saline-treated mice with died prior to day 6, and most mice treated with control tEGFR NK cells died prior to day 7 (FIG. 12D). However, all mice treated with mACE2-CAR_sIL-15 NK cells survived by day 7, and three of the five mice (60%) treated with mACE2-CAR_sIL-15 NK cells recovered and survived until day 12 (FIG. 12D).

To further investigate protection of SARS-CoV-2 infection by mACE2-CAR_sIL-15 NK cells, we used quantitative reverse transcriptase (RT)-PCR to measure viral spike protein RNA levels in the brain and lung of mice infected with 1×10³ PFU live SARS-CoV-2. We found that mice receiving mACE2-CAR_sIL-15 NK cells had significantly lower viral loads of SARS-CoV-2 in brain and lung tissues compared to mice receiving saline or control tEGFR NK cells (FIG. 12E). These data further support our in vivo study with live SARS-CoV-2 that mACE2-CAR-IL-15 NK cells can limit SARS-CoV-2 infection.

### Example 11: Mutated ACE2 Proteins

In this example, we propose the use of a mutated ACE2 protein (mACE2) fused with the CH2 and CH3 domains of a crystallisable fragment (Fc) of an antibody, in order to treat patients suffering from coronavirus infections that utilize a spike protein binding to ACE2 as a critical point of viral entry, including SARS-2-CoV, and thus help to prevent or stop viral spread. The Fc portion can be replaced with another fusion protein or a tag for stabilizing and/or purifying the mACE2 protein. The Fc region can be an IgG1 or IgG4 scaffold because the IgG1 form can be used to treat patients without cytokine release syndrome (CRS), while the IgG4 from can be used to treat patients with or without CRS. The rationale for the selective use of IgG1 or IgG4 is that IgG1 can strongly bind to Fc receptors on monocytes/macrophages to induce Fc-Fc receptor-mediated antibody-dependent cellular phagocytosis (ADCP) and inflammation, while IgG4 weakly binds to the Fc receptors on monocytes/macrophages. Likewise, IgG1 can strongly bind to the Fc receptor CD16 on natural killer (NK) cells to induce Fc-Fc receptor-mediated antibody-dependent cellular cytotoxicity (ADCC) and inflammation, while IgG4 weakly binds to the CD16 receptor on NK cells.

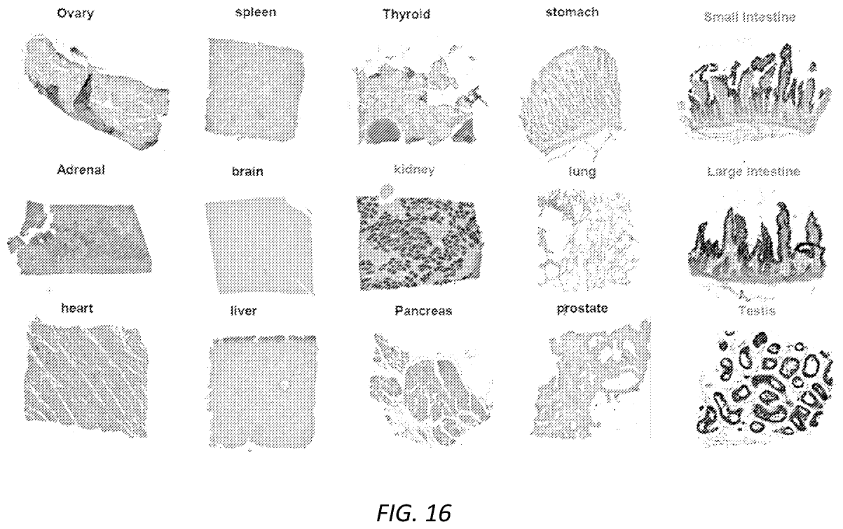

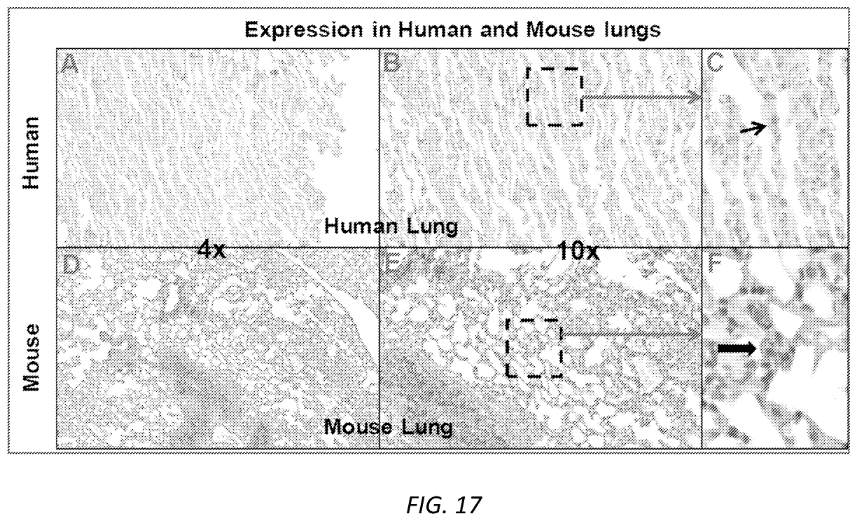

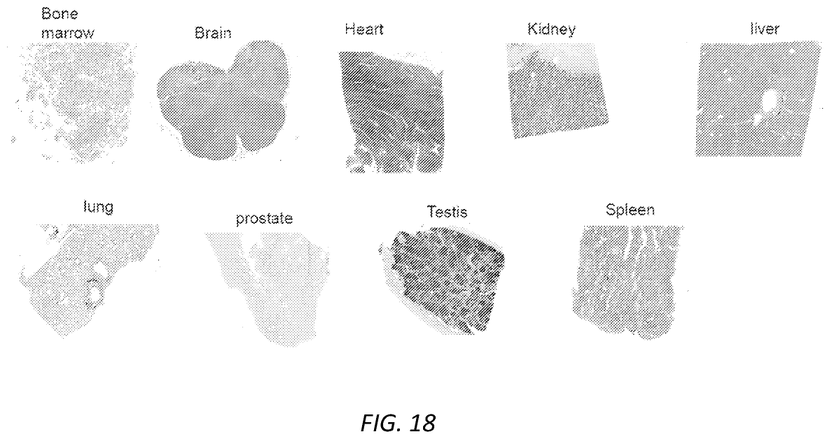

In both SARS-CoV-1 and SARS-CoV-2 infections, the coronavirus spike (S) glycoprotein promotes the coronavirus entry into host cells via the host receptor angiotensin (Ang) converting enzyme 2 (ACE2) (Walls, A. C., Park, Y. J., Tortorici, M. A., Wall, A., McGuire, A. T. & Veesler, D. Structure, Function, and Antigenicity of the SARS-CoV-2 Spike Glycoprotein. Cell (2020)). Using various tissues from uninfected donors, we determined susceptibility of different organs or tissues to SARS-CoV-2 infection via assessment of ACE2 expression by immunohistochemistry (IHC). Interestingly, we observed that lungs only showed a moderate level of ACE2 expression, while expression levels in kidney, small and large intestines, and testis were observed to be very high (FIG. 16). Although the overall ACE2 expression level in the lung samples was moderate, a closer analysis with higher magnification revealed that some cells lining alveoli in both murine and human clearly expressed ACE2 (FIG. 17). These data suggested that SARS-CoV-2 may enter the body through the respiratory tract and attack the lung, but that the virus may also be amplified in organs with high ACE2 expression, which may be the cause for direct and severe damage to these organs. Indeed, the New York Times reported on Apr. 18, 2020, that 20-40% of intensive care unit (I.C.U.) patients with COVID-19 suffered kidney failure and needed emergency dialysis. Using autopsy tissue samples from deceased COVID-19 patients, we observed similar expression levels in the lung, liver, kidney, and testis as that in uninfected individuals, while the expression level of ACE2 in the heart of the deceased COVID-19 patients was noticeably higher than that of uninfected individuals (FIGS. 16 and 18).

Recently published in the journal Cell, Monteil et al. preformed preclinical studies and used ACE2 to successfully block SARS-CoV-2 to enter host cells (Monteil, V., Kwon, H., Prado, P., Hagelkruys, A., Wimmer, R. A., Stahl, M., Leopoldi, A., Garreta, E., Hurtado Del Pozo, C., Prosper, F., Romero, J. P., Wirnsberger, G., Zhang, H., Slutsky, A. S., Conder, R., Montserrat, N., Mirazimi, A. & Penninger, J. M. Inhibition of SARS-CoV-2 Infections in Engineered Human Tissues Using Clinical-Grade Soluble Human ACE2. Cell 181, 905-913 e907 (2020)). However, this might not be an ideal approach and may have safety concerns, as ACE2 normally catalyzes the conversion of Ang II into Ang 1-7, which is a known vasodilator. Consequently, the excessive binding of the ACE2 could lead excessive enzymatic conversion of angiotensin 2 to angiotensin 1-7 and consequent hypotention and cardiovascular collapse (Velkoska, E., Patel, S. K. & Burrell, L. M. Angiotensin converting enzyme 2 and diminazene: role in cardiovascular and blood pressure regulation. Curr. Opin. Nephrol. Hypertens. 25, 384-395 (2016)). Further, the interaction of the spike protein of the coronavirus with the wild type ACE2 in cells through internalization and degradation of the protein. ACE2 has been shown to have a protective effect against virus-induced lung injury by increasing the production of the vasodilator Ang 1-7 (Imai, Y., Kuba, K. & Penninger, J. M. The discovery of angiotensin-converting enzyme 2 and its role in acute lung injury in mice. Exp. Physiol. 93, 543-548 (2008); Jia, H. Pulmonary Angiotensin-Converting Enzyme 2 (ACE2) and Inflammatory Lung Disease. Shock 46, 239-248 (2016)). Thus, reduction of ACE2 via its binding the coronavirus spike protein may have a deleterious effect on the lung, such as that seen in SARS-CoV-1 and SARS-CoV-2 infections. Hence, preventing receptor occupancy of the wild type ACE2 protein by out-competing it for binding to the spike protein with a mutant ACE2 molecule that allows preservation of wild type ACE2's enzymatic conversion of Ang II into Ang 1-7, but itself as a mutant ACE2 molecule does not retain the enzymatic conversion of Ang II into Ang 1-7 should be a rational strategy to be pursued for the prevention and/or treatment of SARS-CoV-2 infection.

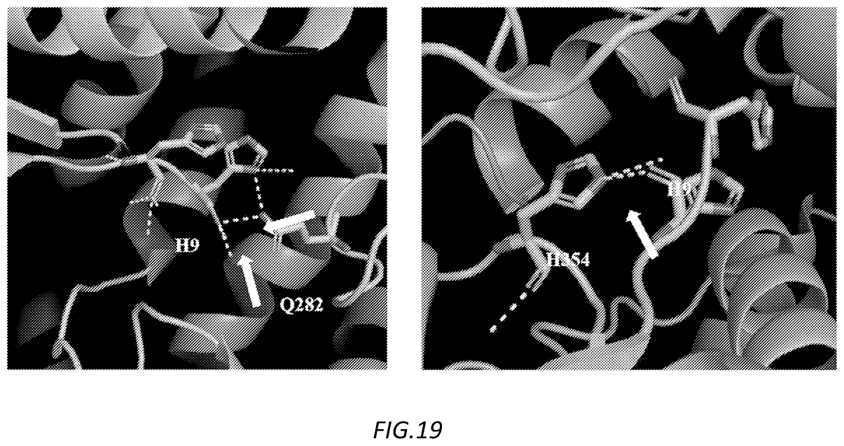

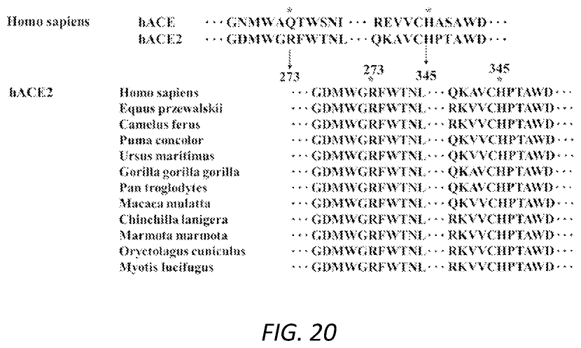

In this example, we mutated (m) the extracellular (e) domain ACE2 with the hope to (1) maintain the receptor's capacity to bind to the spike protein, while (2) disrupting its normal enzymatic conversion of Ang II into Ang 1-7. We took a single nucleotide or amino acid mutation approach to minimize interference of ACE2 binding to the spike protein. ACE2 and ACE are homologous to each other and both can bind to angiotensin-II and use it as a substrate. The structure of the ACE2 and angiotensin-II interaction is unknown, while the structure of ACE and angiotensin-II interaction is known. Using computer modeling, we observed that Q282 and H354 of ACE are the two critical amino acids that interact with H9 of angiotensin-II (FIG. 19). Using sequencing alignment, we observed Q282 and H354 of ACE correspond to R273 and H345 of ACE2 and the regions with these amino acids are generally homologous to each other (FIG. 20). Importantly, R273 and H345 and their flanking regions are highly conserved among 12 species, including Homo sapiens, Equus przewalskii, Camelus ferus, Puma concolor, Ursus maritimus, Gorilla gorilla gorilla, Pan troglodytes, Macaca mulatta, Chinchilla lanigera, Marmota marmota, Oryctolagus cuniculus, and Myotis lucifugus (FIG. 20).

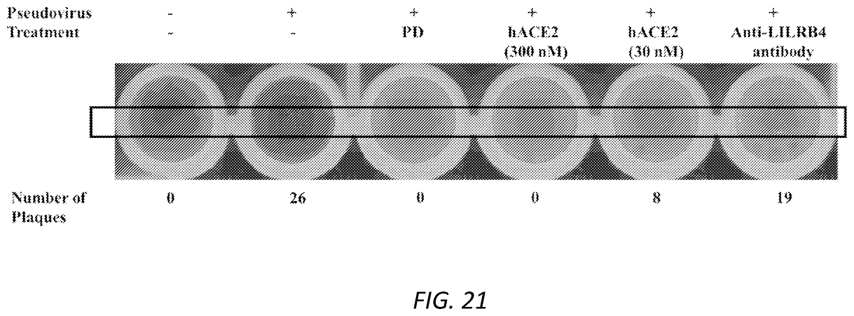

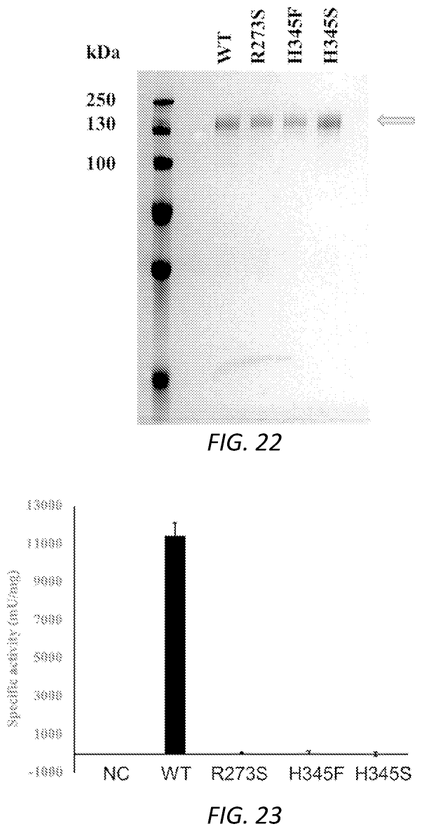

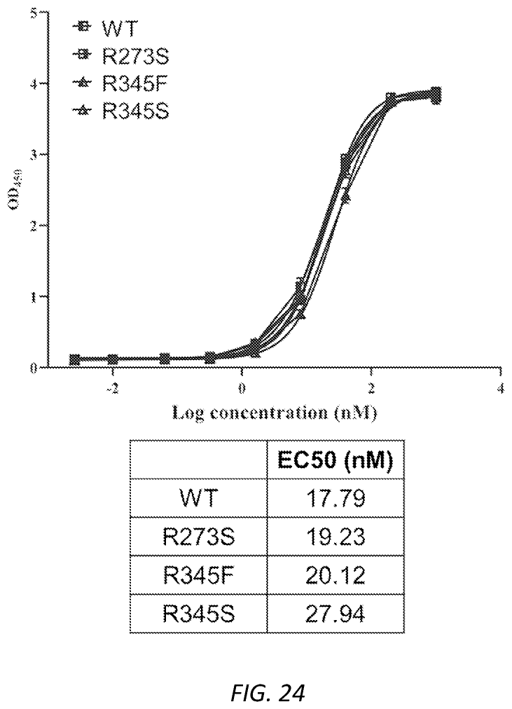

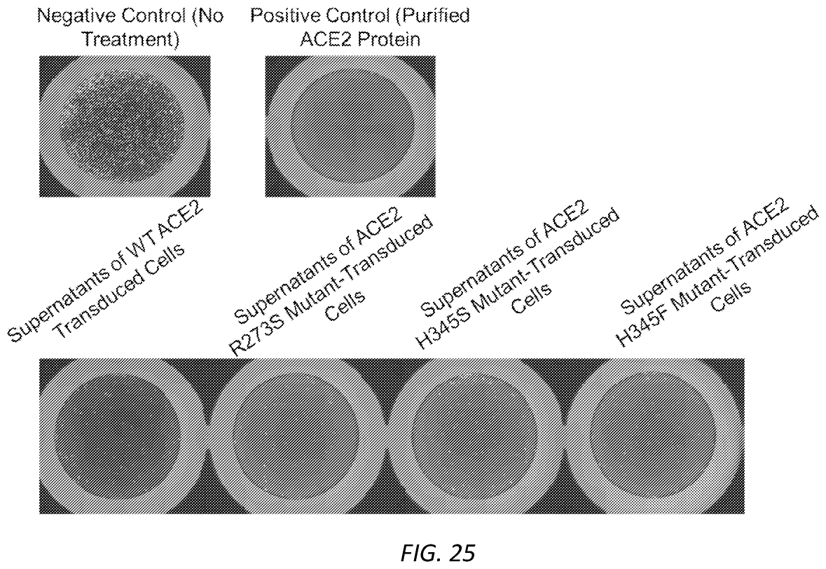

We first cloned wild-type ACE2 and generated the corresponding protein. We observed that ACE2 blocked the entry of SARS-CoV-2 spike pseudotyped VSV virus into Vero cells in a dose dependent manner, with an almost complete blockade effect at approximately 300 nM. The positive control for this assay is plasma from convalescent COVID-19 patients (PD) while the negative control is a non-relevant anti-LILRB4 antibody, both of which functioned as expected (FIG. 21). We then performed a single nucleotide or amino acid mutation of the two key amino acids that we identified, R273 and H345. We generated three mutated ACE2 (mACE2) proteins, R273S, H345F, and H345S and expressed and purified the corresponding mACE2 proteins (FIG. 22). We found all three mACE2 proteins completely lacked the normal enzymatic activity of wild type ACE2, while the wildtype ACE2 showed its usual high enzymatic activity in the same assay (FIG. 23). Importantly, all three mACE2 proteins retained the same level of the binding affinity to the SARS-CoV-2 spike protein as the wild type ACE2 protein (FIG. 24).

Our data suggest that the mACE2 approach of competitively inhibiting the binding the coronavirus spike protein to the wild type ACE2 protein can be seen as a safe and effective approach to treat COVID-19 patients, especially for the population of patient which can develop severe complications from COVID-19. This technology could be applied to treat any other coronavirus infections that utilize a spike protein binding to ACE2 as a critical point of viral entry.

### Example 12: SARS-CoV-2 Receptor Binding Domain (RBD) Neutralizing Antibodies

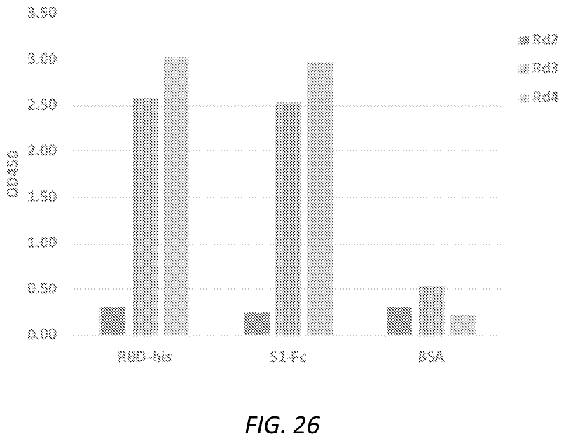

SARS-CoV-2 has caused an unprecedented health care crisis, resulting in unaccountable economic and social loss. SARS (or SARS-CoV-1) and Middle East Respiratory Syndrome (MERS) are also in the family of coronavirus and both previously caused epidemic or pandemics as well. In both SARS-CoV-1 and SARS-CoV-2 infections, the coronavirus spike (S) glycoprotein promotes the coronavirus entry into host cells via the host receptor angiotensin (Ang) converting enzyme 2 (ACE2; Walls, A. C., Park, Y. J., Tortorici, M. A., Wall, A., McGuire, A. T. & Veesler, D. Structure, Function, and Antigenicity of the SARS-CoV-2 Spike Glycoprotein. Cell (2020)) (FIG. 26). Here we undertook a neutralizing antibody approach to treat patients infected with known or unknown viruses such as SARS-CoV-2 expressing the spike protein, aiming to prevent or stop the viral spread.

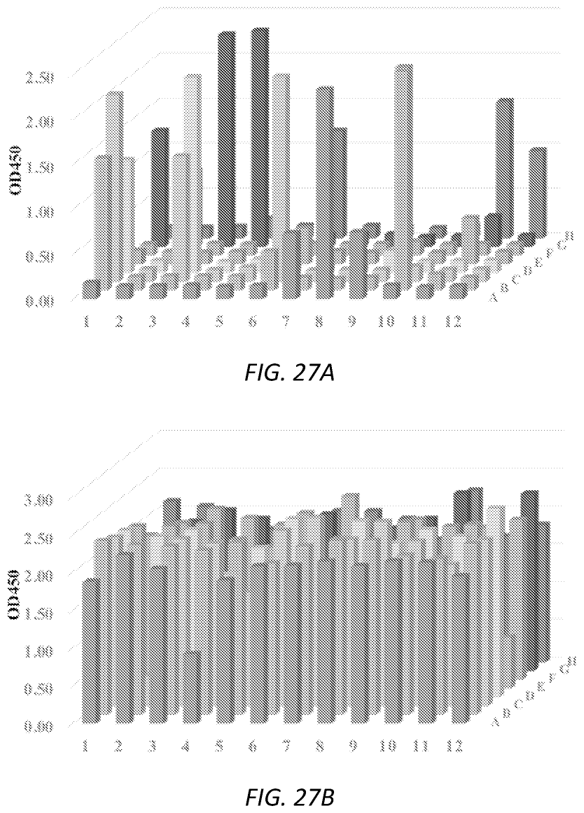

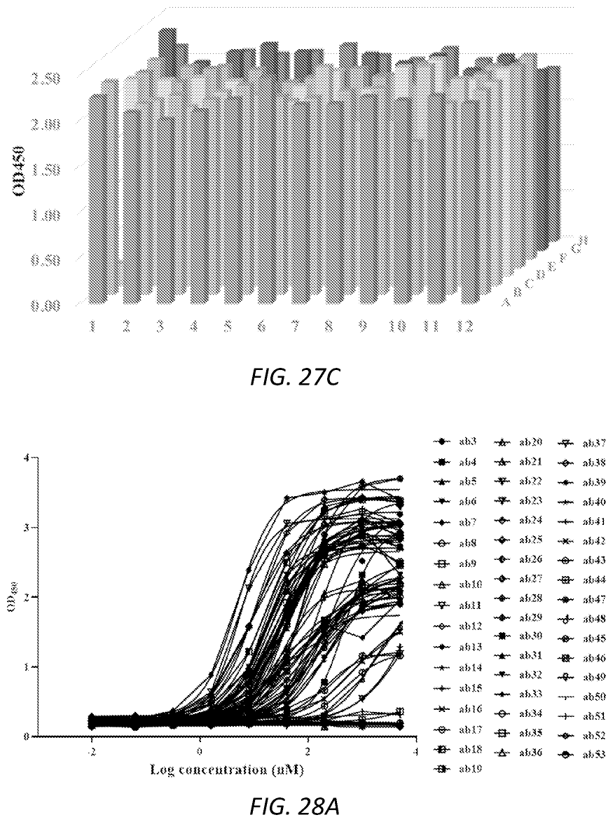

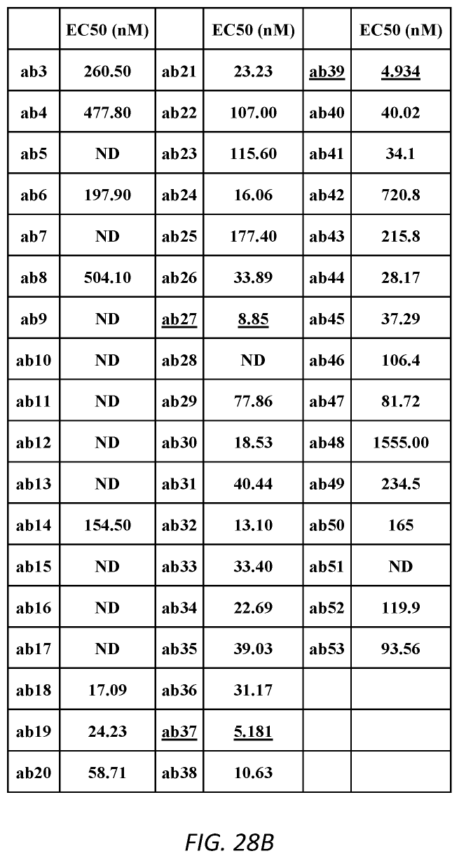

We started with generating a naïve phage single chain antibody (scFv) library using mixed cDNA of B cells isolated from approximately fifty healthy donors. We then cloned the receptor binding domain (RBD) of the spike protein and used it as a bait to screen the library by enzyme-linked immunosorbent assay (ELISA) (FIGS. 27A-27C). We used a Histidine tag (His-tag) to fuse to the RBD so that we could purify screened scFv for functional analysis. The S1 portion of the full-length spike protein fused with CH2 and CH3 domains of an Fc receptor (S1-Fc) was used as positive control while bovine serum albumin (BSA) was used as negative control (FIGS. 27A-27C). Bulk screening showed that using both RBD-His and S1-Fc as baits, candidate antibodies were enriched while this was not observed for BSA (FIGS. 27A-27C). We then assessed single clones and fifty one clones that produced an anti-RBD antibody were successfully identified after 4 rounds (Rd) of ELISA screening (FIGS. 28A-28B). To do so, 96 single clones from the second to fourth round of selection were picked, respectively, and we observed that the percentage of antigen positive clones dramatically increased from the second to third or fourth round of selection.

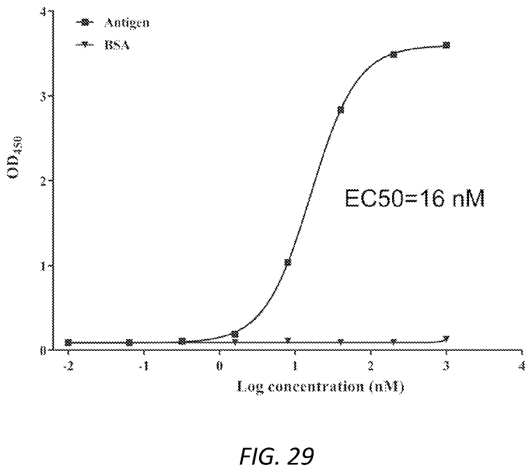

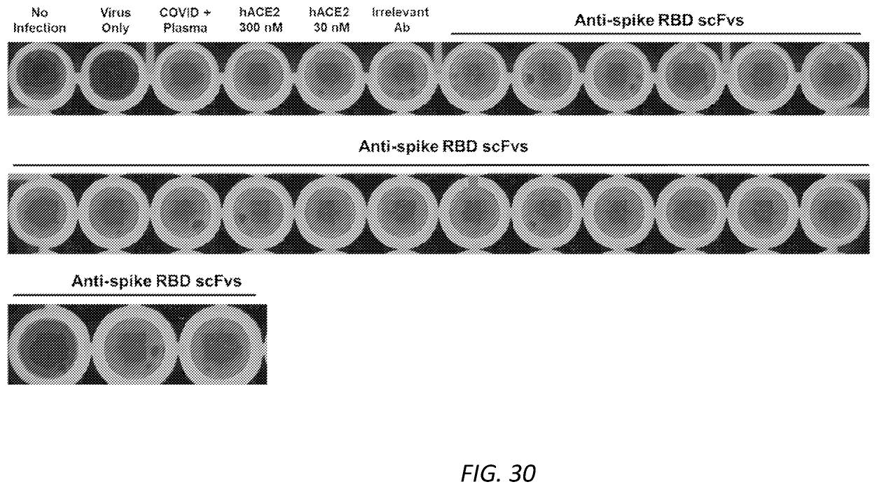

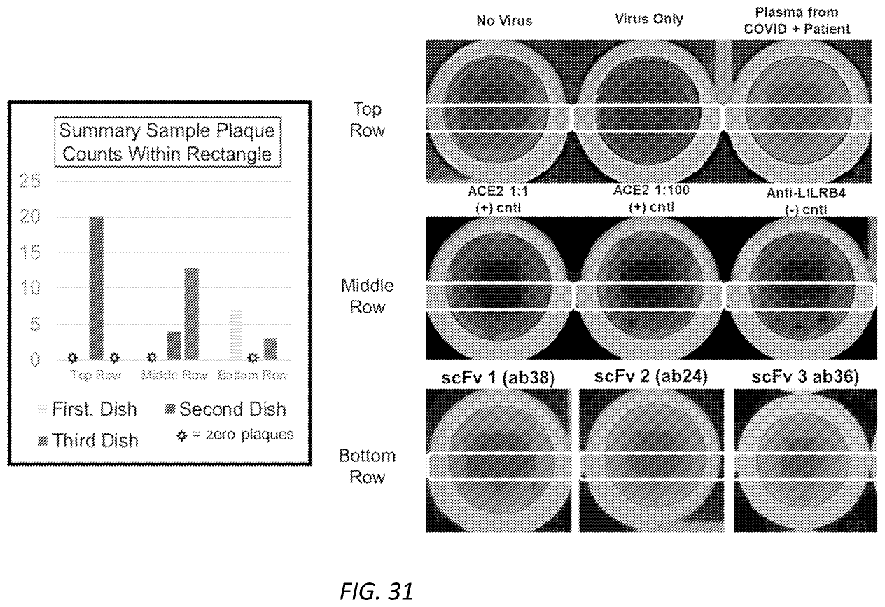

Sanger sequencing analysis of positive clones identified a total of 51 unique clones. These 6× His-tagged clones were subsequently expressed in E. coli cells and purified in scFv format by nickel resin. The half maximal effective concentration (EC₅₀) of the scFvs was determined by ELISA (FIG. 28A). Although these proteins were still single chain variable fragments (scFv), were not very pure, and were not yet full-length antibodies, we identified three clones with a half maximal effective concentration (EC₅₀) below 10 nM. These three clones were 27, 37, and 39, with an EC₅₀ of 8.85 nM, 5.282 nM, 4.934 nM, respectively (FIG. 28B, underlined). These “immature antibodies”, or scFv, were each found to have a higher binding affinity to the spike protein than that of ACE2 (the human receptor that binds the SARS-CoV-2 spike protein), the latter of which had an EC₅₀ of 16 nM (FIG. 29). Using a SARS-CoV-2 spike pseudotyped VSV virus assay, we performed a functional analysis, which indicated that the three scFv that we identified did indeed block the spike protein-mediated viral entry (FIGS. 30-31). Each of the three identified scFv onto an IgG4 or IgG1 scaffold to have a picomolar-scale (pM-scale) binding affinity because the full-length or “mature” antibodies will have a bivalent binding capacity due to their two Fab regions while scFv possess only one Fab for binding. The full-length or mature antibodies should also have an improved in vivo half-life compared to scFv.

Our data suggest that the neutralizing antibodies that we have identified have the potential to effectively treat COVID-19 patients, especially for the populations susceptible to severe complications who are starting to be identified by both racial and ethnic profiling performed by the Centers for Disease Control as well as genomic analyses, on chromosome 3, of a ˜50 kb segment of DNA (www.nytimes.com/interactive/2020/07/05/us/coronavirus-latinos-african-americans-cdc-data.html?action=click&module=Top%20Stories&pgtype=Homepage; Zeberg, H. & Pääbo, S. The major genetic risk factor for severe COVID-19 is inherited from Neandertals. 2020.2007.2003.186296 (2020)). The antibodies disclosed herein can be applied to treat any viral infection that utilize the spike protein to enter host cells.

### REFERENCES

1. N. Zhu, D. Zhang, W. Wang, X. Li, B. Yang, J. Song, X. Zhao, B. Huang, W. Shi, R. Lu, P. Niu, F. Zhan, X. Ma, D. Wang, W. Xu, G. Wu, G. F. Gao, W. Tan, I. China Novel Coronavirus, T. Research, A Novel Coronavirus from Patients with Pneumonia in China, 2019. N Engl Med 382, 727-733 (2020).
2. W. H. Organization.
3. C. D. Spinner, R. L. Gottlieb, G. J. Criner, J. R. Arribas Lopez, A. M. Cattelan, A. Soriano Viladomiu, O. Ogbuagu, P. Malhotra, K. M. Mullane, A. Castagna, L. Y. A. Chai, M. Roestenberg, O. T. Y. Tsang, E. Bernasconi, P. Le Turnier, S. C. Chang, D. SenGupta, R. H. Hyland, A. O. Osinusi, H. Cao, C. Blair, H. Wang, A. Gaggar, D. M. Brainard, M. J. McPhail, S. Bhagani, M. Y. Ahn, A. J. Sanyal, G. Huhn, F. M. Marty, G.-U.-. Investigators, Effect of Remdesivir vs Standard Care on Clinical Status at 11 Days in Patients With Moderate COVID-19: A Randomized Clinical Trial. JAMA 324, 1048-1057 (2020).
4. Y. Wang, D. Zhang, G. Du, R. Du, J. Zhao, Y. Jin, S. Fu, L. Gao, Z. Cheng, Q. Lu, Y. Hu, G. Luo, K. Wang, Y. Lu, H. Li, S. Wang, S. Ruan, C. Yang, C. Mei, Y. Wang, D. Ding, F. Wu, X. Tang, X. Ye, Y. Ye, B. Liu, J. Yang, W. Yin, A. Wang, G. Fan, F. Zhou, Z. Liu, X. Gu, J. Xu, L. Shang, Y. Zhang, L. Cao, T. Guo, Y. Wan, H. Qin, Y. Jiang, T. Jaki, F. G. Hayden, P. W. Horby, B. Cao, C. Wang, Remdesivir in adults with severe COVID-19: a randomised, double-blind, placebo-controlled, multicentre trial. Lancet 395, 1569-1578 (2020).
5. R. C. Group, P. Horby, W. S. Lim, J. R. Emberson, M. Mafham, J. L. Bell, L. Linsell, N. Staplin, C. Brightling, A. Ustianowski, E. Elmahi, B. Prudon, C. Green, T. Felton, D. Chadwick, K. Rege, C. Fegan, L. C. Chappell, S. N. Faust, T. Jaki, K. Jeffery, A. Montgomery, K. Rowan, E. Juszczak, J. K. Baillie, R. Haynes, M. J. Landray, Dexamethasone in Hospitalized Patients with Covid-19—Preliminary Report. N Engl J Med, (2020).
6. W. H. O. S. T. Consortium, H. Pan, R. Peto, A. M. Henao-Restrepo, M. P. Preziosi, V. Sathiyamoorthy, Q. Abdool Karim, M. M. Alejandria, C. Hernandez Garcia, M. P. Kieny, R. Malekzadeh, S. Murthy, K. S. Reddy, M. Roses Periago, P. Abi Hanna, F. Ader, A. M. Al-Bader, A. Alhasawi, E. Allum, A. Alotaibi, C. A. Alvarez-Moreno, S. Appadoo, A. Asiri, P. Aukrust, A. Barratt-Due, S. Bellani, M. Branca, H. B. C. Cappel-Porter, N. Cerrato, T. S. Chow, N. Como, J. Eustace, P. J. Garcia, S. Godbole, E. Gotuzzo, L. Griskevicius, R. Hamra, M. Hassan, M. Hassany, D. Hutton, I. Irmansyah, L. Jancoriene, J. Kirwan, S. Kumar, P. Lennon, G. Lopardo, P. Lydon, N. Magrini, T. Maguire, S. Manevska, O. Manuel, S. McGinty, M. T. Medina, M. L. Mesa Rubio, M. C. Miranda-Montoya, J. Nel, E. P. Nunes, M. Perola, A. Portoles, M. R. Rasmin, A. Raza, H. Rees, P. P. S. Reges, C. A. Rogers, K. Salami, M. I. Salvadori, N. Sinani, J. A. C. Sterne, M. Stevanovikj, E. Tacconelli, K. A. O. Tikkinen, S. Trelle, H. Zaid, J. A. Rottingen, S. Swaminathan, Repurposed Antiviral Drugs for Covid-19—Interim WHO Solidarity Trial Results. N Engl J Med, (2020).
7. R. C. Su, S. K. Kung, J. Gariepy, B. H. Barber, R. G. Miller, NK cells can recognize different forms of class I MHV. J Immunol 161, 755-766 (1998).
8. H. G. Ljunggren, K. Karre, In search of the ‘missing self’: MHC molecules and NK cell recognition. Immunol Today 11, 237-244 (1990).
9. R. Ma, T. Lu, Z. Li, K. Y. Teng, A. G. Mansour, M. Yu, L. Tian, B. Xu, S. Ma, J. Zhang, T. Barr, Y. Peng, M. A. Caligiuri, J. Yu, An oncolytic virus expressing IL-15/IL-15Ralpha combined with off-the-shelf EGFR-CAR NK cells targets glioblastoma. Cancer Res, (2021).
10. A. Yilmaz, H. Cui, M. A. Caligiuri, J. Yu, Chimeric antigen receptor-engineered natural killer cells for cancer immunotherapy. J Hematol Oncol 13, 168 (2020).
11. J. Kurtzberg, A History of Cord Blood Banking and Transplantation. Stem Cells Transl Med 6, 1309-1311 (2017).
12. E. Liu, D. Marin, P. Banerjee, H. A. Macapinlac, P. Thompson, R. Basar, L. Nassif Kerbauy, B. Overman, P. Thall, M. Kaplan, V. Nandivada, I. Kaur, A. Nunez Cortes, K. Cao, M. Daher, C. Hosing, E. N. Cohen, P. Kebriaei, R. Mehta, S. Neelapu, Y. Nieto, M. Wang, W. Wierda, M. Keating, R. Champlin, E. J. Shpall, K. Rezvani, Use of CAR-Transduced Natural Killer Cells in CD19-Positive Lymphoid Tumors. N Engl J Med 382, 545-553 (2020).
13. M. Letko, A. Marzi, V. Munster, Functional assessment of cell entry and receptor usage for SARS-CoV-2 and other lineage B betacoronaviruses. Nat Microbiol 5, 562-569 (2020).
14. D. Wrapp, N. Wang, K. S. Corbett, J. A. Goldsmith, C. L. Hsieh, O. Abiona, B. S. Graham, J. S. McLellan, Cryo-EM structure of the 2019-nCoV spike in the prefusion conformation. Science 367, 1260-1263 (2020).
15. P. J. M. Brouwer, T. G. Caniels, K. van der Straten, J. L. Snitselaar, Y. Aldon, S. Bangaru, J. L. Torres, N. M. A. Okba, M. Claireaux, G. Kerster, A. E. H. Bentlage, M. M. van Haaren, D. Guerra, J. A. Burger, E. E. Schermer, K. D. Verheul, N. van der Velde, A. van der Kooi, J. van Schooten, M. J. van Breemen, T. P. L. Bijl, K. Sliepen, A. Aartse, R. Derking, I. Bontjer, N. A. Kootstra, W. J. Wiersinga, G. Vidarsson, B. L. Haagmans, A. B. Ward, G. J. de Bree, R. W. Sanders, M. J. van Gils, Potent neutralizing antibodies from COVID-19 patients define multiple targets of vulnerability. Science 369, 643-650 (2020).
16. T. F. Rogers, F. Zhao, D. Huang, N. Beutler, A. Burns, W. T. He, O. Limbo, C. Smith, G. Song, J. Woehl, L. Yang, R. K. Abbott, S. Callaghan, E. Garcia, J. Hurtado, M. Parren, L. Peng, S. Ramirez, J. Ricketts, M. J. Ricciardi, S. A. Rawlings, N. C. Wu, M. Yuan, D. M. Smith, D. Nemazee, J. R. Teijaro, J. E. Voss, I. A. Wilson, R. Andrabi, B. Briney, E. Landais, D. Sok, J. G. Jardine, D. R. Burton, Isolation of potent SARS-CoV-2 neutralizing antibodies and protection from disease in a small animal model. Science 369, 956-963 (2020).
17. Y. Wu, F. Wang, C. Shen, W. Peng, D. Li, C. Zhao, Z. Li, S. Li, Y. Bi, Y. Yang, Y. Gong, H. Xiao, Z. Fan, S. Tan, G. Wu, W. Tan, X. Lu, C. Fan, Q. Wang, Y. Liu, C. Zhang, J. Qi, G. F. Gao, F. Gao, L. Liu, A noncompeting pair of human neutralizing antibodies block COVID-19 virus binding to its receptor ACE2. Science 368, 1274-1278 (2020).
18. S. J. Zost, P. Gilchuk, J. B. Case, E. Binshtein, R. E. Chen, J. P. Nkolola, A. Schafer, J. X. Reidy, A. Trivette, R. S. Nargi, R. E. Sutton, N. Suryadevara, D. R. Martinez, L. E. Williamson, E. C. Chen, T. Jones, S. Day, L. Myers, A. O. Hassan, N. M. Kafai, E. S. Winkler, J. M. Fox, S. Shrihari, B. K. Mueller, J. Meiler, A. Chandrashekar, N. B. Mercado, J. J. Steinhardt, K. Ren, Y. M. Loo, N. L. Kallewaard, B. T. McCune, S. P. Keeler, M. J. Holtzman, D. H. Barouch, L. E. Gralinski, R. S. Baric, L. B. Thackray, M. S. Diamond, R. H. Carnahan, J. E. Crowe, Jr., Potently neutralizing and protective human antibodies against SARS-CoV-2. Nature 584, 443-449 (2020).
19. W. B. Alsoussi, J. S. Turner, J. B. Case, H. Zhao, A. J. Schmitz, J. Q. Zhou, R. E. Chen, T. Lei, A. A. Rizk, K. M. McIntire, E. S. Winkler, J. M. Fox, N. M. Kafai, L. B. Thackray, A. O. Hassan, F. Amanat, F. Krammer, C. T. Watson, S. H. Kleinstein, D. H. Fremont, M. S. Diamond, A. H. Ellebedy, A Potently Neutralizing Antibody Protects Mice against SARS-CoV-2 Infection. J Immunol 205, 915-922 (2020).
20. R. Zang, J. B. Case, E. Yutuc, X. Ma, S. Shen, M. F. Gomez Castro, Z. Liu, Q. Zeng, H. Zhao, J. Son, P. W. Rothlauf, A. J. B. Kreutzberger, G. Hou, H. Zhang, S. Bose, X. Wang, M. D. Vahey, K. Mani, W. J. Griffiths, T. Kirchhausen, D. H. Fremont, H. Guo, A. Diwan, Y. Wang, M. S. Diamond, S. P. J. Whelan, S. Ding, Cholesterol 25-hydroxylase suppresses SARS-CoV-2 replication by blocking membrane fusion. Proc Natl Acad Sci USA 117, 32105-32113 (2020).
21. J. Yu, W. Dong, A. Li, J. Zhang, M. Caligiuri, S. Jaramillo, H. L. Mead, N. E. Stone, A. Jones, D. R. Kollath, V. K. Coyne, M. Yearsley, L.-S. Wang, B. M. Barker, P. Keim, T. Barr, The K18-hACE2 Transgenic Mouse Model Recapitulates Non-Severe and Severe COVID-19 in Response to Infectious Dose of SARS-CoV-2 Virus. 2021.2005.2008.443244 (2021).
22. A. O. Hassan, J. B. Case, E. S. Winkler, L. B. Thackray, N. M. Kafai, A. L. Bailey, B. T. McCune, J. M. Fox, R. E. Chen, W. B. Alsoussi, J. S. Turner, A. J. Schmitz, T. Lei, S. Shrihari, S. P. Keeler, D. H. Fremont, S. Greco, P. B. McCray, Jr., S. Perlman, M. J. Holtzman, A. H. Ellebedy, M. S. Diamond, A SARS-CoV-2 Infection Model in Mice Demonstrates Protection by Neutralizing Antibodies. Cell 182, 744-753 e744 (2020).
23. E. S. Winkler, A. L. Bailey, N. M. Kafai, S. Nair, B. T. McCune, J. Yu, J. M. Fox, R. E. Chen, J. T. Earnest, S. P. Keeler, J. H. Ritter, L. I. Kang, S. Dort, A. Robichaud, R. Head, M. J. Holtzman, M. S. Diamond, SARS-CoV-2 infection of human ACE2-transgenic mice causes severe lung inflammation and impaired function. Nat Immunol 21, 1327-1335 (2020).
24. J. W. Wang, J. M. Howson, T. Ghansah, C. Desponts, J. M. Ninos, S. L. May, K. H. Nguyen, N. Toyama-Sorimachi, W. G. Kerr, Influence of SHIP on the NK repertoire and allogeneic bone marrow transplantation. Science 295, 2094-2097 (2002).
25. V. Hoyos, B. Savoldo, C. Quintarelli, A. Mahendravada, M. Zhang, J. Vera, H. E. Heslop, C. M. Rooney, M. K. Brenner, G. Dotti, Engineering CD19-specific T lymphocytes with interleukin-15 and a suicide gene to enhance their anti-lymphoma/leukemia effects and safety. Leukemia 24, 1160-1170 (2010).
26. Y. Tagaya, R. N. Bamford, A. P. DeFilippis, T. A. Waldmann, IL-15: a pleiotropic cytokine with diverse receptor/signaling pathways whose expression is controlled at multiple levels. Immunity 4, 329-336 (1996).
27. C. Sahm, K. Schonfeld, W. S. Wels, Expression of IL-15 in NK cells results in rapid enrichment and selective cytotoxicity of gene-modified effectors that carry a tumor-specific antigen receptor. Cancer Immunol Immunother 61, 1451-1461 (2012).
28. H. S. Dai, N. Griffin, C. Bolyard, H. C. Mao, J. Zhang, T. P. Cripe, T. Suenaga, H. Arase, I. Nakano, E. A. Chiocca, B. Kaur, J. Yu, M. A. Caligiuri, The Fc Domain of Immunoglobulin Is Sufficient to Bridge NK Cells with Virally Infected Cells. Immunity 47, 159-170 e110 (2017).
29. M. Zheng, Y. Gao, G. Wang, G. Song, S. Liu, D. Sun, Y. Xu, Z. Tian, Functional exhaustion of antiviral lymphocytes in COVID-19 patients. Cell Mol Immunol 17, 533-535 (2020).
30. D. Li, Y. Chen, H. Liu, Y. Jia, F. Li, W. Wang, J. Wu, Z. Wan, Y. Cao, R. Zeng, Immune dysfunction leads to mortality and organ injury in patients with COVID-19 in China: insights from ERS-COVID-19 study. Signal Transduct Target Ther 5, 62 (2020).
31. B. Diao, C. Wang, Y. Tan, X. Chen, Y. Liu, L. Ning, L. Chen, M. Li, Y. Liu, G. Wang, Z. Yuan, Z. Feng, Y. Zhang, Y. Wu, Y. Chen, Reduction and Functional Exhaustion of T Cells in Patients With Coronavirus Disease 2019 (COVID-19). Front Immunol 11, 827 (2020).
32. F. Wang, J. Nie, H. Wang, Q. Zhao, Y. Xiong, L. Deng, S. Song, Z. Ma, P. Mo, Y. Zhang, Characteristics of Peripheral Lymphocyte Subset Alteration in COVID-19 Pneumonia. J Infect Dis 221, 1762-1769 (2020).
33. M. K. Kennedy, M. Glaccum, S. N. Brown, E. A. Butz, J. L. Viney, M. Embers, N. Matsuki, K. Charrier, L. Sedger, C. R. Willis, K. Brasel, P. J. Morrissey, K. Stocking, J. C. Schuh, S. Joyce, J. J. Peschon, Reversible defects in natural killer and memory CD8 T cell lineages in interleukin 15-deficient mice. J Exp Med 191, 771-780 (2000).
34. A. W. Goldrath, P. V. Sivakumar, M. Glaccum, M. K. Kennedy, M. J. Bevan, C. Benoist, D. Mathis, E. A. Butz, Cytokine requirements for acute and Basal homeostatic proliferation of naive and memory CD8+ T cells. J Exp Med 195, 1515-1522 (2002).
35. R. Badolato, A. N. Ponzi, M. Millesimo, L. D. Notarangelo, T. Musso, Interleukin-15 (IL-15) induces IL-8 and monocyte chemotactic protein 1 production in human monocytes. Blood 90, 2804-2809 (1997).
36. D. G. Alleva, S. B. Kaser, M. A. Monroy, M. J. Fenton, D. I. Beller, IL-15 functions as a potent autocrine regulator of macrophage proinflammatory cytokine production: evidence for differential receptor subunit utilization associated with stimulation or inhibition. J Immunol 159, 2941-2951 (1997).
37. S. S. Farag, T. A. Fehniger, L. Ruggeri, A. Velardi, M. A. Caligiuri, Natural killer cell receptors: new biology and insights into the graft-versus-leukemia effect. Blood 100, 1935-1947 (2002).
38. L. Ruggeri, M. Capanni, E. Urbani, K. Perruccio, W. D. Shlomchik, A. Tosti, S. Posati, D. Rogaia, F. Frassoni, F. Aversa, M. F. Martelli, A. Velardi, Effectiveness of donor natural killer cell alloreactivity in mismatched hematopoietic transplants. Science 295, 2097-2100 (2002).
39. M. Daher, K. Rezvani, Next generation natural killer cells for cancer immunotherapy: the promise of genetic engineering. Curr Opin Immunol 51, 146-153 (2018).
40. E. C. Morris, S. S. Neelapu, T. Giavridis, M. Sadelain, Cytokine release syndrome and associated neurotoxicity in cancer immunotherapy. Nat Rev Immunol, (2021).
41. C. Huang, Y. Wang, X. Li, L. Ren, J. Zhao, Y. Hu, L. Zhang, G. Fan, J. Xu, X. Gu, Z. Cheng, T. Yu, J. Xia, Y. Wei, W. Wu, X. Xie, W. Yin, H. Li, M. Liu, Y. Xiao, H. Gao, L. Guo, J. Xie, G. Wang, R. Jiang, Z. Gao, Q. Jin, J. Wang, B. Cao, Clinical features of patients infected with 2019 novel coronavirus in Wuhan, China. Lancet 395, 497-506 (2020).
42. C. Qin, L. Zhou, Z. Hu, S. Zhang, S. Yang, Y. Tao, C. Xie, K. Ma, K. Shang, W. Wang, D. S. Tian, Dysregulation of Immune Response in Patients With Coronavirus 2019 (COVID-19) in Wuhan, China. Clin Infect Dis 71, 762-768 (2020).
43. S. F. Pedersen, Y. C. Ho, SARS-CoV-2: a storm is raging. J Clin Invest 130, 2202-2205 (2020).
44. S. K. Tsui, S. S. Chim, Y. M. Lo, S. R. G. Chinese University of Hong Kong Molecular, Coronavirus genomic-sequence variations and the epidemiology of the severe acute respiratory syndrome. N Engl J Med 349, 187-188 (2003).

PARTIAL INFORMAL SEQUENCE LISTING

Nucleic acid sequence of hACE2 WT-long extracellular domain (SEQ ID NO: B1):

| | |
|---|---|
| ATGTCAAGCT CTTCCTGGCT CCTTCTCAGC CTTGTTGCTG TAACTGCTGC TCAGTCCACC | 60 |
| ATTGAGGAAC AGGCCAAGAC ATTTTTGGAC AAGTTTAACC ACGAAGCCGA AGACCTGTTC | 120 |
| TATCAAAGTT CACTTGCTTC TTGGAATTAT AACACCAATA TTACTGAAGA GAATGTCCAA | 180 |
| AACATGAATA ATGCTGGGGA CAAATGGTCT GCCTTTTTAA AGGAACAGTC CACACTTGCC | 240 |
| CAAATGTATC CACTACAAGA AATTCAGAAT CTCACAGTCA AGCTTCAGCT GCAGGCTCTT | 300 |
| CAGCAAAATG GGTCTTCAGT GCTCTCAGAA GACAAGAGCA AACGGTTGAA CACAATTCTA | 360 |
| AATACAATGA GCACCATCTA CAGTACTGGA AAAGTTTGTA ACCCAGATAA TCCACAAGAA | 420 |
| TGCTTATTAC TTGAACCAGG TTTGAATGAA ATAATGGCAA ACAGTTTAGA CTACAATGAG | 480 |
| AGGCTCTGGG CTTGGGAAAG CTGGAGATCT GAGGTCGGCA AGCAGCTGAG GCCATTATAT | 540 |
| GAAGAGTATG TGGTCTTGAA AAATGAGATG GCAAGAGCAA ATCATTATGA GGACTATGGG | 600 |
| GATTATTGGA GAGGAGACTA TGAAGTAAAT GGGGTAGATG GCTATGACTA CAGCCGCGGC | 660 |
| CAGTTGATTG AAGATGTGGA ACATACCTTT GAAGAGATTA AACCATTATA TGAACATCTT | 720 |
| CATGCCTATG TGAGGGCAAA GTTGATGAAT GCCTATCCTT CCTATATCAG TCCAATTGGA | 780 |
| TGCCTCCCTG CTCATTTGCT TGGTGATATG TGGGGTAGAT TTTGGACAAA TCTGTACTCT | 840 |
| TTGACAGTTC CCTTTGGACA GAAACCAAAC ATAGATGTTA CTGATGCAAT GGTGGACCAG | 900 |
| GCCTGGGATG CACAGAGAAT ATTCAAGGAG GCCGAGAAGT TCTTTGTATC TGTTGGTCTT | 960 |
| CCTAATATGA CTCAAGGATT CTGGGAAAAT TCCATGCTAA CGGACCCAGG AAATGTTCAG | 1020 |
| AAAGCAGTCT GCCATCCCAC AGCTTGGGAC CTGGGGAAGG GCGACTTCAG GATCCTTATG | 1080 |
| TGCACAAAGG TGACAATGGA CGACTTCCTG ACAGCTCATC ATGAGATGGG GCATATCCAG | 1140 |
| TATGATATGG CATATGOTGC ACAACCTTTT CTGCTAAGAA ATGGAGCTAA TGAAGGATTC | 1200 |
| CATGAAGCTG TTGGGGAAAT CATGTCACTT TCTGCAGCCA CACCTAAGCA TTTAAAATCC | 1260 |
| ATTGGTCTTC TGTCACCCGA TTTTCAAGAA GACAATGAAA CAGAAATAAA CTTCCTGCTC | 1320 |
| AAACAAGCAC TCACGATTGT TGGGACTCTG CCATTTACTT ACATGTTAGA GAAGTGGAGG | 1380 |
| TGGATGGTCT TTAAAGGGGA AATTCCCAAA GACCAGTGGA TGAAAAAGTG GTGGGAGATG | 1440 |
| AAGCGAGAGA TAGTTGGGGT GGTGGAACCT GTGCCCCATG ATGAAACATA CTGTGACCCC | 1500 |
| GCATCTCTGT TCCATGTTTC TAATGATTAC TCATTCATTC GATATTACAC AAGGACCCTT | 1560 |
| TACCAATTCC AGTTTCAAGA AGCACTTTGT CAAGCAGCTA AACATGAAGG CCCTCTGCAC | 1620 |
| AAATGTGACA TCTCAAACTC TACAGAAGCT GGACAGAAAC TGTTCAATAT GCTGAGGCTT | 1680 |
| GGAAAATCAG AACCCTGGAC CCTAGCATTG GAAAATGTTG TAGGAGCAAA GAACATGAAT | 1740 |
| GTAAGGCCAC TGCTCAACTA CTTTGAGCCC TTATTTACCT GGCTGAAAGA CCAGAACAAG | 1800 |
| AATTCTTTTG TGGGATGGAG TACCGACTGG AGTCCATATG CAGACCAAAG CATCAAAGTG | 1860 |
| AGGATAAGCC TAAAATCAGC TOTTGGAGAT AAAGCATATG AATGGAACGA CAATGAAATG | 1920 |
| TACCTGTTCC GATCATCTGT TGCATATGCT ATGAGGCAGT ACTTTTTAAA AGTAAAAAAT | 1980 |
| CAGATGATTC TTTTTGGGGA GGAGGATGTG CGAGTGGCTA ATTTGAAACC AAGAATCTCC | 2040 |
| TTTAATTTCT TTGTCACTGC ACCTAAAAAT GTGTCTGATA TCATTCCTAG AACTGAAGTT | 2100 |
| GAAAAGGCCA TCAGGATGTC CCGGAGCCGT ATCAATGATG CTTTCCGTCT GAATGACAAC | 2160 |
| AGCCTAGAGT TTCTGGGGAT ACAGCCAACA CTTGGACCTC CTAACCAGCC CCCTGTTTCC | 2220 |

Amino acid sequence of hACE2 WT-long extracellular domain (SEQ ID NO: B2):

| | |
|---|---|
| MSSSSWLLLSLVAVTAAQSTIEEQAKTFLDKFNHEAEDLFYQSSLASWNYNTNITEENVQ | 60 |
| NMNNAGDKWSAFLKEQSTLAQMYPLQEIQNLTVKLQLQALQQNGSSVLSEDKSKRLNTIL | 120 |
| NTMSTIYSTGKVCNPDNPQECLLLEPGLNEIMANSLDYNERLWAWESWRSEVGKQLRPLY | 180 |
| EEYVVLKNEMARANHYEDYGDYWRGDYEVNGVDGYDYSRGQLIEDVEHTFEEIKPLYEHL | 240 |
| HAYVRAKLMNAYPSYISPIGCLPAHLLGDMWGRFWTNLYSLTVPFGQKPNIDVTDAMVDQ | 300 |
| AWDAQRIFKEAEKFFVSVGLPNMTQGFWENSMLTDPGNVQKAVCHPTAWDLGKGDFRILM | 360 |
| CTKVTMDDFLTAHHEMGHIQYDMAYAAQPFLLRNGANEGFHEAVGEIMSLSAATPKHLKS | 420 |
| IGLLSPDFQEDNETEINFLLKQALTIVGTLPFTYMLEKWRWMVFKGEIPKDQWMKKWWEM | 480 |
| KREIVGVVEPVPHDETYCDPASLFHVSNDYSFIRYYTRTLYQFQFQEALCQAAKHEGPLH | 540 |
| KCDISNSTEAGQKLFNMLRLGKSEPWTLALENVVGAKNMNVRPLLNYFEPLFTWLKDQNK | 600 |
| NSFVGWSTDWSPYADQSIKVRISLKSALGDKAYEWNDNEMYLFRSSVAYAMRQYFLKVKN | 660 |
| QMILFGEEDVRVANLKPRISFNFFVTAPKNVSDIIPRTEVEKAIRMSRSRINDAFRLNDN | 720 |
| SLEFLGIQPTLGPPNQPPVS | 740 |

Nucleic acid sequence of hACE2 WT-short extracellular domain (SEQ ID NO: B3):

| | |
|---|---|
| ATGTCAAGCT CTTCCTGGCT CCTTCTCAGC CTTGTTGCTG TAACTGCTGC TCAGTCCACC | 60 |
| ATTGAGGAAC AGGCCAAGAC ATTTTTGGAC AAGTTTAACC ACGAAGCCGA AGACCTGTTC | 120 |
| TATCAAAGTT CACTTGCTTC TTGGAATTAT AACACCAATA TTACTGAAGA GAATGTCCAA | 180 |
| AACATGAATA ATGCTGGGGA CAAATGGTCT GCCTTTTTAA AGGAACAGTC CACACTTGCC | 240 |
| CAAATGTATC CACTACAAGA AATTCAGAAT CTCACAGTCA AGCTTCAGCT GCAGGCTCTT | 300 |
| CAGCAAAATG GGTCTTCAGT GCTCTCAGAA GACAAGAGCA AACGGTTGAA CACAATTCTA | 360 |
| AATACAATGA GCACCATCTA CAGTACTGGA AAAGTTTGTA ACCCAGATAA TCCACAAGAA | 420 |
| TGCTTATTAC TTGAACCAGG TTTGAATGAA ATAATGGCAA ACAGTTTAGA CTACAATGAG | 480 |
| AGGCTCTGGG CTTGGGAAAG CTGGAGATCT GAGGTCGGCA AGCAGCTGAG GCCATTATAT | 540 |
| GAAGAGTATG TGGTCTTGAA AAATGAGATG GCAAGAGCAA ATCATTATGA GGACTATGGG | 600 |
| GATTATTGGA GAGGAGACTA TGAAGTAAAT GGGGTAGATG GCTATGACTA CAGCCGCGGC | 660 |
| CAGTTGATTG AAGATGTGGA ACATACCTTT GAAGAGATTA AACCATTATA TGAACATCTT | 720 |
| CATGCCTATG TGAGGGCAAA GTTGATGAAT GCCTATCCTT CCTATATCAG TCCAATTGGA | 780 |
| TGCCTCCCTG CTCATTTGCT TGGTGATATG TGGGGTAGAT TTTGGACAAA TCTGTACTCT | 840 |
| TTGACAGTTC CCTTTGGACA GAAACCAAAC ATAGATGTTA CTGATGCAAT GGTGGACCAG | 900 |
| GCCTGGGATG CACAGAGAAT ATTCAAGGAG GCCGAGAAGT TCTTTGTATC TGTTGGTCTT | 960 |
| CCTAATATGA CTCAAGGATT CTGGGAAAAT TCCATGCTAA CGGACCCAGG AAATGTTCAG | 1020 |
| AAAGCAGTCT GCCATCCCAC AGCTTGGGAC CTGGGGAAGG GCGACTTCAG GATCCTTATG | 1080 |
| TGCACAAAGG TGACAATGGA CGACTTCCTG ACAGCTCATC ATGAGATGGG GCATATCCAG | 1140 |
| TATGATATGG CATATGCTGC ACAACCTTTT CTGCTAAGAA ATGGAGCTAA TGAAGGATTC | 1200 |
| CATGAAGCTG TTGGGGAAAT CATGTCACTT TCTGCAGCCA CACCTAAGCA TTTAAAATCC | 1260 |
| ATTGGTCTTC TGTCACCCGA TTTTCAAGAA GACAATGAAA CAGAAATAAA CTTCCTGCTC | 1320 |
| AAACAAGCAC TCACGATTGT TGGGACTCTG CCATTTACTT ACATGTTAGA GAAGTGGAGG | 1380 |
| TGGATGGTCT TTAAAGGGGA AATTCCCAAA GACCAGTGGA TGAAAAAGTG GTGGGAGATG | 1440 |
| AAGCGAGAGA TAGTTGGGGT GGTGGAACCT GTGCCCCATG ATGAAACATA CTGTGACCCC | 1500 |
| GCATCTCTGT TCCATGTTTC TAATGATTAC TCATTCATTC GATATTACAC AAGGACCCTT | 1560 |
| TACCAATTCC AGTTTCAAGA AGCACTTTGT CAAGCAGCTA AACATGAAGG CCCTCTGCAC | 1620 |
| AAATGTGACA TCTCAAACTC TACAGAAGCT GGACAGAAAC TGTTCAATAT GCTGAGGCTT | 1680 |
| GGAAAATCAG AACCCTGGAC CCTAGCATTG GAAAATGTTG TAGGAGCAAA GAACATGAAT | 1740 |
| GTAAGGCCAC TGCTCAACTA CTTTGAGCCC TTATTTACCT GGCTGAAAGA CCAGAACAAG | 1800 |
| AATTCTTTTG TGGGATGGAG TACCGACTGG AGTCCATATG CAGAC | 1845 |

Amino acid sequence of hACE2 WT-short extracellular domain (SEQ ID NO: B4):

| | |
|---|---|
| MSSSSWLLLSLVAVTAAQSTIEEQAKTFLDKFNHEAEDLFYQSSLASWNYNTNITEENVQ | 60 |
| NMNNAGDKWSAFLKEQSTLAQMYPLQEIQNLTVKLQLQALQQNGSSVLSEDKSKRLNTIL | 120 |
| NTMSTIYSTGKVCNPDNPQECLLLEPGLNEIMANSLDYNERLWAWESWRSEVGKQLRPLY | 180 |
| EEYVVLKNEMARANHYEDYGDYWRGDYEVNGVDGYDYSRGQLIEDVEHTFEEIKPLYEHL | 240 |
| HAYVRAKLMNAYPSYISPIGCLPAHLLGDMWGRFWTNLYSLTVPFGQKPNIDVTDAMVDQ | 300 |
| AWDAQRIFKEAEKFFVSVGLPNMTQGFWENSMLTDPGNVQKAVCHPTAWDLGKGDFRILM | 360 |
| CTKVTMDDFLTAHHEMGHIQYDMAYAAQPFLLRNGANEGFHEAVGEIMSLSAATPKHLKS | 420 |
| IGLLSPDFQEDNETEINFLLKQALTIVGTLPFTYMLEKWRWMVFKGEIPKDQWMKKWWEM | 480 |
| KREIVGVVEPVPHDETYCDPASLFHVSNDYSFIRYYTRTLYQFQFQEALCQAAKHEGPLH | 540 |
| KCDISNSTEAGQKLFNMLRLGKSEPWTLALENVVGAKNMNVRPLLNYFEPLFTWLKDQNK | 600 |
| NSFVGWSTDWSPYAD | 615 |

Nucleic acid sequence of hACE2 R273S (SEQ ID NO: B5):

| | |
|---|---|
| ATGTCAAGCT CTTCCTGGCT CCTTCTCAGC CTTGTTGCTG TAACTGCTGC TCAGTCCACC | 60 |
| ATTGAGGAAC AGGCCAAGAC ATTTTTGGAC AAGTTTAACC ACGAAGCCGA AGACCTGTTC | 120 |
| TATCAAAGTT CACTTGCTTC TTGGAATTAT AACACCAATA TTACTGAAGA GAATGTCCAA | 180 |
| AACATGAATA ATGCTGGGGA CAAATGGTCT GCCTTTTTAA AGGAACAGTC CACACTTGCC | 240 |
| CAAATGTATC CACTACAAGA AATTCAGAAT CTCACAGTCA AGCTTCAGCT GCAGGCTCTT | 300 |
| CAGCAAAATG GGTCTTCAGT GCTCTCAGAA GACAAGAGCA AACGGTTGAA CACAATTCTA | 360 |
| AATACAATGA GCACCATCTA CAGTACTGGA AAAGTTTGTA ACCCAGATAA TCCACAAGAA | 420 |
| TGCTTATTAC TTGAACCAGG TTTGAATGAA ATAATGGCAA ACAGTTTAGA CTACAATGAG | 480 |
| AGGCTCTGGG CTTGGGAAAG CTGGAGATCT GAGGTCGGCA AGCAGCTGAG GCCATTATAT | 540 |
| GAAGAGTATG TGGTCTTGAA AAATGAGATG GCAAGAGCAA ATCATTATGA GGACTATGGG | 600 |
| GATTATTGGA GAGGAGACTA TGAAGTAAAT GGGGTAGATG GCTATGACTA CAGCCGCGGC | 660 |
| CAGTTGATTG AAGATGTGGA ACATACCTTT GAAGAGATTA AACCATTATA TGAACATCTT | 720 |
| CATGCCTATG TGAGGGCAAA GTTGATGAAT GCCTATCCTT CCTATATCAG TCCAATTGGA | 780 |
| TGCCTCCCTG CTCATTTGCT TGGTGATATG TGGGGTAGCT TTTGGACAAA TCTGTACTCT | 840 |
| TTGACAGTTC CCTTTGGACA GAAACCAAAC ATAGATGTTA CTGATGCAAT GGTGGACCAG | 900 |
| GCCTGGGATG CACAGAGAAT ATTCAAGGAG GCCGAGAAGT TCTTTGTATC TGTTGGTCTT | 960 |
| CCTAATATGA CTCAAGGATT CTGGGAAAAT TCCATGCTAA CGGACCCAGG AAATGTTCAG | 1020 |
| AAAGCAGTCT GCCATCCCAC AGCTTGGGAC CTGGGGAAGG GCGACTTCAG GATCCTTATG | 1080 |
| TGCACAAAGG TGACAATGGA CGACTTCCTG ACAGCTCATC ATGAGATGGG GCATATCCAG | 1140 |
| TATGATATGG CATATGCTGC ACAACCTTTT CTGCTAAGAA ATGGAGCTAA TGAAGGATTC | 1200 |
| CATGAAGCTG TTGGGGAAAT CATGTCACTT TCTGCAGCCA CACCTAAGCA TTTAAAATCC | 1260 |
| ATTGGTCTTC TGTCACCCGA TTTTCAAGAA GACAATGAAA CAGAAATAAA CTTCCTGCTC | 1320 |
| AAACAAGCAC TCACGATTGT TGGGACTCTG CCATTTACTT ACATGTTAGA GAAGTGGAGG | 1380 |
| TGGATGGTCT TTAAAGGGGA AATTCCCAAA GACCAGTGGA TGAAAAAGTG GTGGGAGATG | 1440 |
| AAGCGAGAGA TAGTTGGGGT GGTGGAACCT GTGCCCCATG ATGAAACATA CTGTGACCCC | 1500 |
| GCATCTCTGT TCCATGTTTC TAATGATTAC TCATTCATTC GATATTACAC AAGGACCCTT | 1560 |
| TACCAATTCC AGTTTCAAGA AGCACTTTGT CAAGCAGCTA AACATGAAGG CCCTCTGCAC | 1620 |
| AAATGTGACA TCTCAAACTC TACAGAAGCT GGACAGAAAC TGTTCAATAT GCTGAGGCTT | 1680 |
| GGAAAATCAG AACCCTGGAC CCTAGCATTG GAAAATGTTG TAGGAGCAAA GAACATGAAT | 1740 |
| GTAAGGCCAC TGCTCAACTA CTTTGAGCCC TTATTTACCT GGCTGAAAGA CCAGAACAAG | 1800 |
| AATTCTTTTG TGGGATGGAG TACCGACTGG AGTCCATATG CAGACCAAAG CATCAAAGTG | 1860 |
| AGGATAAGCC TAAAATCAGC TOTTGGAGAT AAAGCATATG AATGGAACGA CAATGAAATG | 1920 |
| TACCTGTTCC GATCATCTGT TGCATATGCT ATGAGGCAGT ACTTTTTAAA AGTAAAAAAT | 1980 |
| CAGATGATTC TTTTTGGGGA GGAGGATGTG CGAGTGGCTA ATTTGAAACC AAGAATCTCC | 2040 |
| TTTAATTTCT TTGTCACTGC ACCTAAAAAT GTGTCTGATA TCATTCCTAG AACTGAAGTT | 2100 |
| GAAAAGGCCA TCAGGATGTC CCGGAGCCGT ATCAATGATG CTTTCCGTCT GAATGACAAC | 2160 |
| AGCCTAGAGT TTCTGGGGAT ACAGCCAACA CTTGGACCTC CTAACCAGCC CCCTGTTTCC | 2220 |

Amino acid sequence of hACE2 R273S (SEQ ID NO: B6):

| | |
|---|---|
| MSSSSWLLLSLVAVTAAQSTIEEQAKTFLDKFNHEAEDLFYQSSLASWNYNTNITEENVQ | 60 |
| NMNNAGDKWSAFLKEQSTLAQMYPLQEIQNLTVKLQLQALQQNGSSVLSEDKSKRLNTIL | 120 |
| NTMSTIYSTGKVCNPDNPQECLLLEPGLNEIMANSLDYNERLWAWESWRSEVGKQLRPLY | 180 |
| EEYVVLKNEMARANHYEDYGDYWRGDYEVNGVDGYDYSRGQLIEDVEHTFEEIKPLYEHL | 240 |
| HAYVRAKLMNAYPSYISPIGCLPAHLLGDMWGSFWTNLYSLTVPFGQKPNIDVTDAMVDQ | 300 |
| AWDAQRIFKEAEKFFVSVGLPNMTQGFWENSMLTDPGNVQKAVCHPTAWDLGKGDFRILM | 360 |
| CTKVTMDDFLTAHHEMGHIQYDMAYAAQPFLLRNGANEGFHEAVGEIMSLSAATPKHLKS | 420 |
| IGLLSPDFQEDNETEINFLLKQALTIVGTLPFTYMLEKWRWMVFKGEIPKDQWMKKWWEM | 480 |
| KREIVGVVEPVPHDETYCDPASLFHVSNDYSFIRYYTRTLYQFQFQEALCQAAKHEGPLH | 540 |
| KCDISNSTEAGQKLFNMLRLGKSEPWTLALENVVGAKNMNVRPLLNYFEPLFTWLKDQNK | 600 |
| NSFVGWSTDWSPYADQSIKVRISLKSALGDKAYEWNDNEMYLFRSSVAYAMRQYFLKVKN | 660 |
| QMILFGEEDVRVANLKPRISFNFFVTAPKNVSDIIPRTEVEKAIRMSRSRINDAFRLNDN | 720 |
| SLEFLGIQPTLGPPNQPPVS | 740 |

Nucleic acid sequence of hACE2 H345F (SEQ ID NO: B7):

| | |
|---|---|
| ATGTCAAGCT CTTCCTGGCT CCTTCTCAGC CTTGTTGCTG TAACTGCTGC TCAGTCCACC | 60 |
| ATTGAGGAAC AGGCCAAGAC ATTTTTGGAC AAGTTTAACC ACGAAGCCGA AGACCTGTTC | 120 |
| TATCAAAGTT CACTTGCTTC TTGGAATTAT AACACCAATA TTACTGAAGA GAATGTCCAA | 180 |
| AACATGAATA ATGCTGGGGA CAAATGGTCT GCCTTTTTAA AGGAACAGTC CACACTTGCC | 240 |
| CAAATGTATC CACTACAAGA AATTCAGAAT CTCACAGTCA AGCTTCAGCT GCAGGCTCTT | 300 |
| CAGCAAAATG GGTCTTCAGT GCTCTCAGAA GACAAGAGCA AACGGITGAA CACAATTCTA | 360 |
| AATACAATGA GCACCATCTA CAGTACTGGA AAAGTTTGTA ACCCAGATAA TCCACAAGAA | 420 |
| TGCTTATTAC TTGAACCAGG TTTGAATGAA ATAATGGCAA ACAGTTTAGA CTACAATGAG | 480 |
| AGGCTCTGGG CTTGGGAAAG CTGGAGATCT GAGGTCGGCA AGCAGCTGAG GCCATTATAT | 540 |
| GAAGAGTATG TGGTCTTGAA AAATGAGATG GCAAGAGCAA ATCATTATGA GGACTATGGG | 600 |
| GATTATTGGA GAGGAGACTA TGAAGTAAAT GGGGTAGATG GCTATGACTA CAGCCGCGGC | 660 |
| CAGTTGATTG AAGATGTGGA ACATACCTTT GAAGAGATTA AACCATTATA TGAACATCTT | 720 |
| CATGCCTATG TGAGGGCAAA GTTGATGAAT GCCTATCCTT CCTATATCAG TCCAATTGGA | 780 |
| TGCCTCCCTG CTCATTTGCT TGGTGATATG TGGGGTAGAT TTTGGACAAA TCTGTACTCT | 840 |
| TTGACAGTTC CCTTTGGACA GAAACCAAAC ATAGATGTTA CTGATGCAAT GGTGGACCAG | 900 |
| GCCTGGGATG CACAGAGAAT ATTCAAGGAG GCCGAGAAGT TCTTTGTATC TGTTGGTCTT | 960 |
| CCTAATATGA CTCAAGGATT CTGGGAAAAT TCCATGCTAA CGGACCCAGG AAATGTTCAG | 1020 |
| AAAGCAGTCT GCTTTCCCAC AGCTTGGGAC CTGGGGAAGG GCGACTTCAG GATCCTTATG | 1080 |
| TGCACAAAGG TGACAATGGA CGACTTCCTG ACAGCTCATC ATGAGATGGG GCATATCCAG | 1140 |
| TATGATATGG CATATGCTGC ACAACCTTTT CTGCTAAGAA ATGGAGCTAA TGAAGGATTC | 1200 |
| CATGAAGCTG TTGGGGAAAT CATGTCACTT TCTGCAGCCA CACCTAAGCA TTTAAAATCC | 1260 |
| ATTGGTCTTC TGTCACCCGA TTTTCAAGAA GACAATGAAA CAGAAATAAA CTTCCTGCTC | 1320 |
| AAACAAGCAC TCACGATTGT TGGGACTCTG CCATTTACTT ACATGTTAGA GAAGTGGAGG | 1380 |
| TGGATGGTCT TTAAAGGGGA AATTCCCAAA GACCAGTGGA TGAAAAAGTG GTGGGAGATG | 1440 |
| AAGCGAGAGA TAGTTGGGGT GGTGGAACCT GTGCCCCATG ATGAAACATA CTGTGACCCC | 1500 |
| GCATCTCTGT TCCATGTTTC TAATGATTAC TCATTCATTC GATATTACAC AAGGACCCTT | 1560 |
| TACCAATTCC AGTTTCAAGA AGCACTTTGT CAAGCAGCTA AACATGAAGG CCCTCTGCAC | 1620 |
| AAATGTGACA TCTCAAACTC TACAGAAGCT GGACAGAAAC TGTTCAATAT GCTGAGGCTT | 1680 |
| GGAAAATCAG AACCCTGGAC CCTAGCATTG GAAAATGTTG TAGGAGCAAA GAACATGAAT | 1740 |
| GTAAGGCCAC TGCTCAACTA CTTTGAGCCC TTATTTACCT GGCTGAAAGA CCAGAACAAG | 1800 |
| AATTCTTTTG TGGGATGGAG TACCGACTGG AGTCCATATG CAGACCAAAG CATCAAAGTG | 1860 |
| AGGATAAGCC TAAAATCAGC TOTTGGAGAT AAAGCATATG AATGGAACGA CAATGAAATG | 1920 |
| TACCTGTTCC GATCATCTGT TGCATATGCT ATGAGGCAGT ACTTTTTAAA AGTAAAAAAT | 1980 |
| CAGATGATTC TTTTTGGGGA GGAGGATGTG CGAGTGGCTA ATTTGAAACC AAGAATCTCC | 2040 |
| TTTAATTTCT TTGTCACTGC ACCTAAAAAT GTGTCTGATA TCATTCCTAG AACTGAAGTT | 2100 |
| GAAAAGGCCA TCAGGATGTC CCGGAGCCGT ATCAATGATG CTTTCCGTCT GAATGACAAC | 2160 |
| AGCCTAGAGT TTCTGGGGAT ACAGCCAACA CTTGGACCTC CTAACCAGCC CCCTGTTTCC | 2220 |

Amino acid sequence of hACE2 H345F (SEQ ID NO: B8):

| | |
|---|---|
| MSSSSWLLLSLVAVTAAQSTIEEQAKTFLDKFNHEAEDLFYQSSLASWNYNTNITEENVQ | 60 |
| NMNNAGDKWSAFLKEQSTLAQMYPLQEIQNLTVKLQLQALQQNGSSVLSEDKSKRLNTIL | 120 |
| NTMSTIYSTGKVCNPDNPQECLLLEPGLNEIMANSLDYNERLWAWESWRSEVGKQLRPLY | 180 |
| EEYVVLKNEMARANHYEDYGDYWRGDYEVNGVDGYDYSRGQLIEDVEHTFEEIKPLYEHL | 240 |
| HAYVRAKLMNAYPSYISPIGCLPAHLLGDMWGRFWTNLYSLTVPFGQKPNIDVTDAMVDQ | 300 |
| AWDAQRIFKEAEKFFVSVGLPNMTQGFWENSMLTDPGNVQKAVCFPTAWDLGKGDFRILM | 360 |
| CTKVTMDDFLTAHHEMGHIQYDMAYAAQPFLLRNGANEGFHEAVGEIMSLSAATPKHLKS | 420 |
| IGLLSPDFQEDNETEINFLLKQALTIVGTLPFTYMLEKWRWMVFKGEIPKDQWMKKWWEM | 480 |
| KREIVGVVEPVPHDETYCDPASLFHVSNDYSFIRYYTRTLYQFQFQEALCQAAKHEGPLH | 540 |
| KCDISNSTEAGQKLFNMLRLGKSEPWTLALENVVGAKNMNVRPLLNYFEPLFTWLKDQNK | 600 |
| NSFVGWSTDWSPYADQSIKVRISLKSALGDKAYEWNDNEMYLFRSSVAYAMRQYFLKVKN | 660 |
| QMILFGEEDVRVANLKPRISFNFFVTAPKNVSDIIPRTEVEKAIRMSRSRINDAFRLNDN | 720 |
| SLEFLGIQPTLGPPNQPPVS | 740 |

Nucleic acid sequence of hACE2 H345S (SEQ ID NO: B9):

| | |
|---|---|
| ATGTCAAGCT CTTCCTGGCT CCTTCTCAGC CTTGTTGCTG TAACTGCTGC TCAGTCCACC | 60 |
| ATTGAGGAAC AGGCCAAGAC ATTTTTGGAC AAGTTTAACC ACGAAGCCGA AGACCTGTTC | 120 |
| TATCAAAGTT CACTTGCTTC TTGGAATTAT AACACCAATA TTACTGAAGA GAATGTCCAA | 180 |
| AACATGAATA ATGCTGGGGA CAAATGGTCT GCCTTTTTAA AGGAACAGTC CACACTTGCC | 240 |
| CAAATGTATC CACTACAAGA AATTCAGAAT CTCACAGTCA AGCTTCAGCT GCAGGCTCTT | 300 |
| CAGCAAAATG GGTCTTCAGT GCTCTCAGAA GACAAGAGCA AACGGTTGAA CACAATTCTA | 360 |
| AATACAATGA GCACCATCTA CAGTACTGGA AAAGTTTGTA ACCCAGATAA TCCACAAGAA | 420 |
| TGCTTATTAC TTGAACCAGG TTTGAATGAA ATAATGGCAA ACAGTTTAGA CTACAATGAG | 480 |
| AGGCTCTGGG CTTGGGAAAG CTGGAGATCT GAGGTCGGCA AGCAGCTGAG GCCATTATAT | 540 |
| GAAGAGTATG TGGTCTTGAA AAATGAGATG GCAAGAGCAA ATCATTATGA GGACTATGGG | 600 |
| GATTATTGGA GAGGAGACTA TGAAGTAAAT GGGGTAGATG GCTATGACTA CAGCCGCGGC | 660 |
| CAGTTGATTG AAGATGTGGA ACATACCTTT GAAGAGATTA AACCATTATA TGAACATCTT | 720 |
| CATGCCTATG TGAGGGCAAA GTTGATGAAT GCCTATCCTT CCTATATCAG TCCAATTGGA | 780 |
| TGCCTCCCTG CTCATTTGCT TGGTGATATG TGGGGTAGAT TTTGGACAAA TCTGTACTCT | 840 |
| TTGACAGTTC CCTTTGGACA GAAACCAAAC ATAGATGTTA CTGATGCAAT GGTGGACCAG | 900 |
| GCCTGGGATG CACAGAGAAT ATTCAAGGAG GCCGAGAAGT TCTTTGTATC TGTTGGTCTT | 960 |
| CCTAATATGA CTCAAGGATT CTGGGAAAAT TCCATGCTAA CGGACCCAGG AAATGTTCAG | 1020 |
| AAAGCAGTCT GCTCTCCCAC AGCTTGGGAC CTGGGGAAGG GCGACTTCAG GATCCTTATG | 1080 |
| TGCACAAAGG TGACAATGGA CGACTTCCTG ACAGCTCATC ATGAGATGGG GCATATCCAG | 1140 |
| TATGATATGG CATATGCTGC ACAACCTTTT CTGCTAAGAA ATGGAGCTAA TGAAGGATTC | 1200 |
| CATGAAGCTG TTGGGGAAAT CATGTCACTT TCTGCAGCCA CACCTAAGCA TTTAAAATCC | 1260 |
| ATTGGTCTTC TGTCACCCGA TTTTCAAGAA GACAATGAAA CAGAAATAAA CTTCCTGCTC | 1320 |
| AAACAAGCAC TCACGATTGT TGGGACTCTG CCATTTACTT ACATGTTAGA GAAGTGGAGG | 1380 |
| TGGATGGTCT TTAAAGGGGA AATTCCCAAA GACCAGTGGA TGAAAAAGTG GTGGGAGATG | 1440 |
| AAGCGAGAGA TAGTTGGGGT GGTGGAACCT GTGCCCCATG ATGAAACATA CTGTGACCCC | 1500 |
| GCATCTCTGT TCCATGTTTC TAATGATTAC TCATTCATTC GATATTACAC AAGGACCCTT | 1560 |
| TACCAATTCC AGTTTCAAGA AGCACTTTGT CAAGCAGCTA AACATGAAGG CCCTCTGCAC | 1620 |
| AAATGTGACA TCTCAAACTC TACAGAAGCT GGACAGAAAC TGTTCAATAT GCTGAGGCTT | 1680 |
| GGAAAATCAG AACCCTGGAC CCTAGCATTG GAAAATGTTG TAGGAGCAAA GAACATGAAT | 1740 |
| GTAAGGCCAC TGCTCAACTA CTTTGAGCCC TTATTTACCT GGCTGAAAGA CCAGAACAAG | 1800 |
| AATTCTTTTG TGGGATGGAG TACCGACTGG AGTCCATATG CAGACCAAAG CATCAAAGTG | 1860 |
| AGGATAAGCC TAAAATCAGC TOTTGGAGAT AAAGCATATG AATGGAACGA CAATGAAATG | 1920 |
| TACCTGTTCC GATCATCTGT TGCATATGCT ATGAGGCAGT ACTTTTTAAA AGTAAAAAAT | 1980 |
| CAGATGATTC TTTTTGGGGA GGAGGATGTG CGAGTGGCTA ATTTGAAACC AAGAATCTCC | 2040 |
| TTTAATTTCT TTGTCACTGC ACCTAAAAAT GTGTCTGATA TCATTCCTAG AACTGAAGTT | 2100 |
| GAAAAGGCCA TCAGGATGTC CCGGAGCCGT ATCAATGATG CTTTCCGTCT GAATGACAAC | 2160 |
| AGCCTAGAGT TTCTGGGGAT ACAGCCAACA CTTGGACCTC CTAACCAGCC CCCTGTTTCC | 2220 |

Amino acid sequence of hACE2 H345S (SEQ ID NO: B10)

| | |
|---|---|
| MSSSSWLLLSLVAVTAAQSTIEEQAKTFLDKFNHEAEDLFYQSSLASWNYNTNITEENVQ | 60 |
| NMNNAGDKWSAFLKEQSTLAQMYPLQEIQNLTVKLQLQALQQNGSSVLSEDKSKRLNTIL | 120 |
| NTMSTIYSTGKVCNPDNPQECLLLEPGLNEIMANSLDYNERLWAWESWRSEVGKQLRPLY | 180 |
| EEYVVLKNEMARANHYEDYGDYWRGDYEVNGVDGYDYSRGQLIEDVEHTFEEIKPLYEHL | 240 |
| HAYVRAKLMNAYPSYISPIGCLPAHLLGDMWGRFWTNLYSLTVPFGQKPNIDVTDAMVDQ | 300 |
| AWDAQRIFKEAEKFFVSVGLPNMTQGFWENSMLTDPGNVQKAVCSPTAWDLGKGDFRILM | 360 |
| CTKVTMDDFLTAHHEMGHIQYDMAYAAQPFLLRNGANEGFHEAVGEIMSLSAATPKHLKS | 420 |
| IGLLSPDFQEDNETEINFLLKQALTIVGTLPFTYMLEKWRWMVFKGEIPKDQWMKKWWEM | 480 |
| KREIVGVVEPVPHDETYCDPASLFHVSNDYSFIRYYTRTLYQFQFQEALCQAAKHEGPLH | 540 |
| KCDISNSTEAGQKLFNMLRLGKSEPWTLALENVVGAKNMNVRPLLNYFEPLFTWLKDQNK | 600 |
| NSFVGWSTDWSPYADQSIKVRISLKSALGDKAYEWNDNEMYLFRSSVAYAMRQYFLKVKN | 660 |
| QMILFGEEDVRVANLKPRISFNFFVTAPKNVSDIIPRTEVEKAIRMSRSRINDAFRLNDN | 720 |
| SLEFLGIQPTLGPPNQPPVS | 740 |

Nucleic acid of hIgG4 Fc (SEQ ID NO: B11)

| | |
|---|---|
| GAGTCCAAAT ATGGTCCCCC ATGCCCACCC TGCCCAGCAC CTGAGTTCGA AGGGGGACCA | 60 |
| TCAGTCTTCC TGTTCCCCCC AAAACCCAAG GACACTCTCA TGATCTCCCG GACCCCTGAG | 120 |
| GTCACGTGCG TGGTGGTGGA CGTGAGCCAG GAAGACCCCG AGGTCCAGTT CAACTGGTAC | 180 |
| GTGGATGGCG TGGAGGTGCA TAATGCCAAG ACAAAGCCGC GGGAGGAGCA GTTCAACAGC | 240 |
| ACGTACCGTG TGGTCAGCGT CCTCACCGTC CTGCACCAGG ACTGGCTGAA CGGCAAGGAG | 300 |
| TACAAGTGCA AGGTCTCCAA CAAAGGCCTC CCGTCCTCCA TCGAGAAAAC CATCTCCAAA | 360 |
| GCCAAAGGGC AGCCCCGAGA GCCACAGGTG TACACCCTGC CCCCATCCCA GGAGGAGATG | 420 |
| ACCAAGAACC AGGTCAGCCT GACCTGCCTG GTCAAAGGCT TCTACCCCAG CGACATCGCC | 480 |
| GTGGAGTGGG AGAGCAATGG GCAGCCGGAG AACAACTACA AGACCACGCC TCCCGTGCTG | 540 |
| GACTCCGACG GCTCCTTCTT CCTCTACAGC AGGCTAACCG TGGACAAGAG CAGGTGGCAG | 600 |
| GAGGGGAATG TCTTCTCATG CTCCGTGATG CATGAGGCTC TGCACAACCA CTACACACAG | 660 |
| AAGAGCCTCT CCCTGTCTCT GGGTAAAGCT AGC | 693 |

Amino acid of hIgG4 Fc (SEQ ID NO: B12)

| | |
|---|---|
| ESKYGPPCPPCPAPEFEGGPSVFLFPPKPKDTLMISRTPEVTCVVVDVSQEDPEVQFNWY | 60 |
| VDGVEVHNAKTKPREEQFNSTYRVVSVLTVLHQDWLNGKEYKCKVSNKGLPSSIEKTISK | 120 |
| AKGQPREPQVYTLPPSQEEMTKNQVSLTCLVKGFYPSDIAVEWESNGQPENNYKTTPPVL | 180 |
| DSDGSFFLYSRLTVDKSRWQEGNVFSCSVMHEALHNHYTQKSLSLSLGKAS | 231 |

Nucleic acid of hIgG1 Fc (SEQ ID NO: B13)

| | |
|---|---|
| TCTTCCGACA AAACTCACAC AAGCCCACCG TCACCAGCAC CTGAACTCCT GGGGGGACCG | 60 |
| TCAGTCTTCC TCTTCCCCCC AAAACCCAAG GACACCCTCA TGATCTCCCG GACCCCTGAG | 120 |
| GTCACATGCG TGGTGGTGGA CGTGAGCCAC GAAGACCCTG AGGTCAAGTT CAACTGGTAC | 180 |
| GTGGACGGCG TGGAGGTGCA TAATGCCAAG ACAAAGCCGC GGGAGGAGCA GTACAACAGC | 240 |
| ACGTACCGTG TGGTCAGCGT CCTCACCGTC CTGCACCAGG ACTGGCTGAA TGGCAAGGAG | 300 |
| TACAAGTGCA AGGTCTCCAA CAAAGCCCTC CCAGCCCCCA TCGAGAAAAC CATCTCCAAA | 360 |
| GCCAAAGGGC AGCCCCGAGA ACCACAGGTG TACACCCTGC CCCCATCCCG GGATGAGCTG | 420 |
| ACCAAGAACC AGGTCAGCCT GACCTGCCTG GTCAAAGGCT TCTATCCCAG CGACATCGCC | 480 |
| GTGGAGTGGG AGAGCAATGG GCAGCCGGAG AACAACTACA AGACCACGCC TCCCGTGCTG | 540 |
| GACTCCGACG GCTCCTTCTT CCTCTACAGC AAGCTCACCG TGGACAAGAG CAGGTGGCAG | 600 |
| CAGGGGAACG TCTTCTCATG CTCCGTGATG CATGAGGCTC TGCACAACCA CTACACGCAG | 660 |
| AAGAGCCTCT CCCTGTCTCC GGGTAAA | 687 |

Amino acid of hIgG1 Fc (SEQ ID NO: B14)

| | |
|---|---|
| SSDKTHTSPPSPAPELLGGPSVFLFPPKPKDTLMISRTPEVTCVVVDVSHEDPEVKFNWY | 60 |
| VDGVEVHNAKTKPREEQYNSTYRVVSVLTVLHQDWLNGKEYKCKVSNKALPAPIEKTISK | 120 |
| AKGQPREPQVYTLPPSRDELTKNQVSLTCLVKGFYPSDIAVEWESNGQPENNYKTTPPVL | 180 |
| DSDGSFFLYSKLTVDKSRWQQGNVFSCSVMHEALHNHYTQKSLSLSPGK | 229 |

Nucleic acid sequence of hACE2 WT-short extracellular domain R273S (SEQ ID NO: B15)

| | |
|---|---|
| ATGTCAAGCT CTTCCTGGCT CCTTCTCAGC CTTGTTGCTG TAACTGCTGC TCAGTCCACC | 60 |
| ATTGAGGAAC AGGCCAAGAC ATTTTTGGAC AAGTTTAACC ACGAAGCCGA AGACCTGTTC | 120 |
| TATCAAAGTT CACTTGCTTC TTGGAATTAT AACACCAATA TTACTGAAGA GAATGTCCAA | 180 |
| AACATGAATA ATGCTGGGGA CAAATGGTCT GCCTTTTTAA AGGAACAGTC CACACTTGCC | 240 |
| CAAATGTATC CACTACAAGA AATTCAGAAT CTCACAGTCA AGCTTCAGCT GCAGGCTCTT | 300 |
| CAGCAAAATG GGTCTTCAGT GCTCTCAGAA GACAAGAGCA AACGGITGAA CACAATTCTA | 360 |
| AATACAATGA GCACCATCTA CAGTACTGGA AAAGTTTGTA ACCCAGATAA TCCACAAGAA | 420 |
| TGCTTATTAC TTGAACCAGG TTTGAATGAA ATAATGGCAA ACAGTTTAGA CTACAATGAG | 480 |
| AGGCTCTGGG CTTGGGAAAG CTGGAGATCT GAGGTCGGCA AGCAGCTGAG GCCATTATAT | 540 |
| GAAGAGTATG TGGTCTTGAA AAATGAGATG GCAAGAGCAA ATCATTATGA GGACTATGGG | 600 |
| GATTATTGGA GAGGAGACTA TGAAGTAAAT GGGGTAGATG GCTATGACTA CAGCCGCGGC | 660 |
| CAGTTGATTG AAGATGTGGA ACATACCTTT GAAGAGATTA AACCATTATA TGAACATCTT | 720 |
| CATGCCTATG TGAGGGCAAA GTTGATGAAT GCCTATCCTT CCTATATCAG TCCAATTGGA | 780 |
| TGCCTCCCTG CTCATTTGCT TGGTGATATG TGGGGTAGCT TTTGGACAAA TCTGTACTCT | 840 |
| TTGACAGTTC CCTTTGGACA GAAACCAAAC ATAGATGTTA CTGATGCAAT GGTGGACCAG | 900 |
| GCCTGGGATG CACAGAGAAT ATTCAAGGAG GCCGAGAAGT TCTTTGTATC TGTTGGTCTT | 960 |
| CCTAATATGA CTCAAGGATT CTGGGAAAAT TCCATGCTAA CGGACCCAGG AAATGTTCAG | 1020 |
| AAAGCAGTCT GCCATCCCAC AGCTTGGGAC CTGGGGAAGG GCGACTTCAG GATCCTTATG | 1080 |
| TGCACAAAGG TGACAATGGA CGACTTCCTG ACAGCTCATC ATGAGATGGG GCATATCCAG | 1140 |
| TATGATATGG CATATGOTGC ACAACCTTTT CTGCTAAGAA ATGGAGCTAA TGAAGGATTC | 1200 |
| CATGAAGCTG TTGGGGAAAT CATGTCACTT TCTGCAGCCA CACCTAAGCA TTTAAAATCC | 1260 |
| ATTGGTCTTC TGTCACCCGA TTTTCAAGAA GACAATGAAA CAGAAATAAA CTTCCTGCTC | 1320 |
| AAACAAGCAC TCACGATTGT TGGGACTCTG CCATTTACTT ACATGTTAGA GAAGTGGAGG | 1380 |
| TGGATGGTCT TTAAAGGGGA AATTCCCAAA GACCAGTGGA TGAAAAAGTG GTGGGAGATG | 1440 |
| AAGCGAGAGA TAGITGGGGT GGTGGAACCT GTGCCCCATG ATGAAACATA CTGTGACCCC | 1500 |
| GCATCTCTGT TCCATGTTTC TAATGATTAC TCATTCATTC GATATTACAC AAGGACCCTT | 1560 |
| TACCAATTCC AGTTTCAAGA AGCACTTTGT CAAGCAGCTA AACATGAAGG CCCTCTGCAC | 1620 |
| AAATGTGACA TCTCAAACTC TACAGAAGCT GGACAGAAAC TGTTCAATAT GCTGAGGCTT | 1680 |
| GGAAAATCAG AACCCTGGAC CCTAGCATTG GAAAATGTTG TAGGAGCAAA GAACATGAAT | 1740 |
| GTAAGGCCAC TGCTCAACTA CTTTGAGCCC TTATTTACCT GGCTGAAAGA CCAGAACAAG | 1800 |
| AATTCTTTTG TGGGATGGAG TACCGACTGG AGTCCATATG CAGAC | 1845 |

Amino acid sequence of hACE2 WT-short extracellular domain R273S (SEQ ID NO: B16)

| | |
|---|---|
| MSSSSWLLLSLVAVTAAQSTIEEQAKTFLDKFNHEAEDLFYQSSLASWNYNTNITEENVQ | 60 |
| NMNNAGDKWSAFLKEQSTLAQMYPLQEIQNLTVKLQLQALQQNGSSVLSEDKSKRLNTIL | 120 |
| NTMSTTYSTGKVCNPDNPQECLLLEPGLNEIMANSLDYNERLWAWESWRSEVGKQLRPLY | 180 |
| EEYVVLKNEMARANHYEDYGDYWRGDYEVNGVDGYDYSRGQLIEDVEHTFEEIKPLYEHL | 240 |
| HAYVRAKLMNAYPSYISPIGCLPAHLLGDMWGSFWTNLYSLTVPFGQKPNIDVTDAMVDQ | 300 |
| AWDAQRIFKEAEKFFVSVGLPNMTQGFWENSMLTDPGNVQKAVCHPTAWDLGKGDFRILM | 360 |
| CTKVTMDDFLTAHHEMGHIQYDMAYAAQPFLLRNGANEGFHEAVGEIMSLSAATPKHLKS | 420 |
| IGLLSPDFQEDNETEINFLLKQALTIVGTLPFTYMLEKWRWMVFKGEIPKDQWMKKWWEM | 480 |
| KREIVGVVEPVPHDETYCDPASLFHVSNDYSFIRYYTRTLYQFQFQEALCQAAKHEGPLH | 540 |
| KCDISNSTEAGQKLFNMLRLGKSEPWTLALENVVGAKNMNVRPLLNYFEPLFTWLKDQNK | 600 |
| NSFVGWSTDWSPYAD | 615 |

Nucleic acid sequence of hACE2 WT-short extracellular domain H345F (SEQ ID NO: B17)

| | |
|---|---|
| ATGTCAAGCT CTTCCTGGCT CCTTCTCAGC CTTGTTGCTG TAACTGCTGC TCAGTCCACC | 60 |
| ATTGAGGAAC AGGCCAAGAC ATTTTTGGAC AAGTTTAACC ACGAAGCCGA AGACCTGTTC | 120 |
| TATCAAAGTT CACTTGCTTC TTGGAATTAT AACACCAATA TTACTGAAGA GAATGTCCAA | 180 |
| AACATGAATA ATGOTGGGGA CAAATGGTCT GCCTTTTTAA AGGAACAGTC CACACTTGCC | 240 |
| CAAATGTATC CACTACAAGA AATTCAGAAT CTCACAGTCA AGCTTCAGCT GCAGGCTCTT | 300 |
| CAGCAAAATG GGTCTTCAGT GCTCTCAGAA GACAAGAGCA AACGGTTGAA CACAATTCTA | 360 |
| AATACAATGA GCACCATCTA CAGTACTGGA AAAGTTTGTA ACCCAGATAA TCCACAAGAA | 420 |
| TGCTTATTAC TTGAACCAGG TTTGAATGAA ATAATGGCAA ACAGTTTAGA CTACAATGAG | 480 |
| AGGCTCTGGG CTTGGGAAAG CTGGAGATCT GAGGTCGGCA AGCAGCTGAG GCCATTATAT | 540 |
| GAAGAGTATG TGGTCTTGAA AAATGAGATG GCAAGAGCAA ATCATTATGA GGACTATGGG | 600 |
| GATTATTGGA GAGGAGACTA TGAAGTAAAT GGGGTAGATG GCTATGACTA CAGCCGCGGC | 660 |
| CAGTTGATTG AAGATGTGGA ACATACCTTT GAAGAGATTA AACCATTATA TGAACATCTT | 720 |
| CATGCCTATG TGAGGGCAAA GTTGATGAAT GCCTATCCTT CCTATATCAG TCCAATTGGA | 780 |
| TGCCTCCCTG CTCATTTGCT TGGTGATATG TGGGGTAGAT TITGGACAAA TCTGTACTCT | 840 |
| TTGACAGTTC CCTTTGGACA GAAACCAAAC ATAGATGTTA CTGATGCAAT GGTGGACCAG | 900 |
| GCCTGGGATG CACAGAGAAT ATTCAAGGAG GCCGAGAAGT TCTTTGTATC TGTTGGTCTT | 960 |
| CCTAATATGA CTCAAGGATT CTGGGAAAAT TCCATGCTAA CGGACCCAGG AAATGTTCAG | 1020 |
| AAAGCAGTCT GCTTTCCCAC AGCTTGGGAC CTGGGGAAGG GCGACTTCAG GATCCTTATG | 1080 |
| TGCACAAAGG TGACAATGGA CGACTTCCTG ACAGCTCATC ATGAGATGGG GCATATCCAG | 1140 |
| TATGATATGG CATATGCTGC ACAACCTTTT CTGCTAAGAA ATGGAGCTAA TGAAGGATTC | 1200 |
| CATGAAGCTG TTGGGGAAAT CATGTCACTT TCTGCAGCCA CACCTAAGCA TTTAAAATCC | 1260 |
| ATTGGTCTTC TGTCACCCGA TTTTCAAGAA GACAATGAAA CAGAAATAAA CTTCCTGCTC | 1320 |
| AAACAAGCAC TCACGATTGT TGGGACTCTG CCATTTACTT ACATGTTAGA GAAGTGGAGG | 1380 |
| TGGATGGTCT TTAAAGGGGA AATTCCCAAA GACCAGTGGA TGAAAAAGTG GTGGGAGATG | 1440 |
| AAGCGAGAGA TAGITGGGGT GGTGGAACCT GTGCCCCATG ATGAAACATA CTGTGACCCC | 1500 |
| GCATCTCTGT TCCATGTTTC TAATGATTAC TCATTCATTC GATATTACAC AAGGACCCTT | 1560 |
| TACCAATTCC AGTTTCAAGA AGCACTTIGT CAAGCAGCTA AACATGAAGG CCCTCTGCAC | 1620 |
| AAATGTGACA TCTCAAACTC TACAGAAGCT GGACAGAAAC TGTTCAATAT GCTGAGGCTT | 1680 |
| GGAAAATCAG AACCCTGGAC CCTAGCATTG GAAAATGTTG TAGGAGCAAA GAACATGAAT | 1740 |
| GTAAGGCCAC TGCTCAACTA CTTTGAGCCC TTATTTACCT GGCTGAAAGA CCAGAACAAG | 1800 |
| AATTCTTTTG TGGGATGGAG TACCGACTGG AGTCCATATG CAGAC | 1845 |

Amino acid sequence of hACE2 WT-short extracellular domain H345F (SEQ ID NO: B18)

| | |
|---|---|
| MSSSSWLLLSLVAVTAAQSTIEEQAKTFLDKFNHEAEDLFYQSSLASWNYNTNITEENVQ | 60 |
| NMNNAGDKWSAFLKEQSTLAQMYPLQEIQNLTVKLQLQALQQNGSSVLSEDKSKRLNTIL | 120 |
| NTMSTTYSTGKVCNPDNPQECLLLEPGLNEIMANSLDYNERLWAWESWRSEVGKQLRPLY | 180 |
| EEYVVLKNEMARANHYEDYGDYWRGDYEVNGVDGYDYSRGQLIEDVEHTFEEIKPLYEHL | 240 |
| HAYVRAKLMNAYPSYISPIGCLPAHLLGDMWGRFWTNLYSLTVPFGQKPNIDVTDAMVDQ | 300 |
| AWDAQRIFKEAEKFFVSVGLPNMTQGFWENSMLTDPGNVQKAVCFPTAWDLGKGDFRILM | 360 |
| CTKVTMDDFLTAHHEMGHIQYDMAYAAQPFLLRNGANEGFHEAVGEIMSLSAATPKHLKS | 420 |
| IGLLSPDFQEDNETEINFLLKQALTIVGTLPFTYMLEKWRWMVFKGEIPKDQWMKKWWEM | 480 |
| KREIVGVVEPVPHDETYCDPASLFHVSNDYSFIRYYTRTLYQFQFQEALCQAAKHEGPLH | 540 |
| KCDISNSTEAGQKLFNMLRLGKSEPWTLALENVVGAKNMNVRPLLNYFEPLFTWLKDQNK | 600 |
| NSFVGWSTDWSPYAD | 615 |

Nucleic acid sequence of hACE2 WT-short extracellular domain H345S (SEQ ID NO: B19)

| | |
|---|---|
| ATGTCAAGCT CTTCCTGGCT CCTTCTCAGC CTTGTTGCTG TAACTGCTGC TCAGTCCACC | 60 |
| ATTGAGGAAC AGGCCAAGAC ATTTTTGGAC AAGTTTAACC ACGAAGCCGA AGACCTGTTC | 120 |
| TATCAAAGTT CACTTGCTTC TTGGAATTAT AACACCAATA TTACTGAAGA GAATGTCCAA | 180 |
| AACATGAATA ATGCTGGGGA CAAATGGTCT GCCTTTTTAA AGGAACAGTC CACACTTGCC | 240 |
| CAAATGTATC CACTACAAGA AATTCAGAAT CTCACAGTCA AGOTTCAGCT GCAGGCTCTT | 300 |
| CAGCAAAATG GGTCTTCAGT GCTCTCAGAA GACAAGAGCA AACGGTTGAA CACAATTCTA | 360 |
| AATACAATGA GCACCATCTA CAGTACTGGA AAAGTTTGTA ACCCAGATAA TCCACAAGAA | 420 |
| TGCTTATTAC TTGAACCAGG TTTGAATGAA ATAATGGCAA ACAGTTTAGA CTACAATGAG | 480 |
| AGGCTCTGGG CTTGGGAAAG CTGGAGATCT GAGGTCGGCA AGCAGCTGAG GCCATTATAT | 540 |
| GAAGAGTATG TGGTCTTGAA AAATGAGATG GCAAGAGCAA ATCATTATGA GGACTATGGG | 600 |
| GATTATTGGA GAGGAGACTA TGAAGTAAAT GGGGTAGATG GCTATGACTA CAGCCGCGGC | 660 |
| CAGTTGATTG AAGATGTGGA ACATACCTTT GAAGAGATTA AACCATTATA TGAACATCTT | 720 |
| CATGCCTATG TGAGGGCAAA GTTGATGAAT GCCTATCCTT CCTATATCAG TCCAATTGGA | 780 |
| TGCCTCCCTG CTCATTTGCT TGGTGATATG TGGGGTAGAT TITGGACAAA TCTGTACTCT | 840 |
| TTGACAGTTC CCTTTGGACA GAAACCAAAC ATAGATGTTA CTGATGCAAT GGTGGACCAG | 900 |
| GCCTGGGATG CACAGAGAAT ATTCAAGGAG GCCGAGAAGT TCTTTGTATC TGTTGGTCTT | 960 |
| CCTAATATGA CTCAAGGATT CTGGGAAAAT TCCATGCTAA CGGACCCAGG AAATGTTCAG | 1020 |
| AAAGCAGTCT GCTCTCCCAC AGCTTGGGAC CTGGGGAAGG GCGACTTCAG GATCCTTATG | 1080 |
| TGCACAAAGG TGACAATGGA CGACTTCCTG ACAGCTCATC ATGAGATGGG GCATATCCAG | 1140 |
| TATGATATGG CATATGCTGC ACAACCTTTT CTGCTAAGAA ATGGAGCTAA TGAAGGATTC | 1200 |
| CATGAAGCTG TTGGGGAAAT CATGTCACTT TCTGCAGCCA CACCTAAGCA TTTAAAATCC | 1260 |
| ATTGGTCTTC TGTCACCCGA TTTTCAAGAA GACAATGAAA CAGAAATAAA CTTCCTGCTC | 1320 |
| AAACAAGCAC TCACGATTGT TGGGACTCTG CCATTTACTT ACATGTTAGA GAAGTGGAGG | 1380 |
| TGGATGGTCT TTAAAGGGGA AATTCCCAAA GACCAGTGGA TGAAAAAGTG GTGGGAGATG | 1440 |
| AAGCGAGAGA TAGITGGGGT GGTGGAACCT GTGCCCCATG ATGAAACATA CTGTGACCCC | 1500 |
| GCATCTCTGT TCCATGTTTC TAATGATTAC TCATTCATTC GATATTACAC AAGGACCCTT | 1560 |
| TACCAATTCC AGTTTCAAGA AGCACTTTGT CAAGCAGCTA AACATGAAGG CCCTCTGCAC | 1620 |
| AAATGTGACA TCTCAAACTC TACAGAAGCT GGACAGAAAC TGTTCAATAT GCTGAGGCTT | 1680 |
| GGAAAATCAG AACCCTGGAC CCTAGCATTG GAAAATGTTG TAGGAGCAAA GAACATGAAT | 1740 |
| GTAAGGCCAC TGCTCAACTA CTTTGAGCCC TTATTTACCT GGCTGAAAGA CCAGAACAAG | 1800 |
| AATTCTTTTG TGGGATGGAG TACCGACTGG AGTCCATATG CAGAC | 1845 |

Amino acid sequence of hACE2 WT-short extracellular domain H345S (SEQ ID NO: B20)

| | |
|---|---|
| MSSSSWLLLSLVAVTAAQSTIEEQAKTFLDKFNHEAEDLFYQSSLASWNYNTNITEENVQ | 60 |
| NMNNAGDKWSAFLKEQSTLAQMYPLQEIQNLTVKLQLQALQQNGSSVLSEDKSKRLNTIL | 120 |
| NTMSTTYSTGKVCNPDNPQECLLLEPGLNEIMANSLDYNERLWAWESWRSEVGKQLRPLY | 180 |
| EEYVVLKNEMARANHYEDYGDYWRGDYEVNGVDGYDYSRGQLIEDVEHTFEEIKPLYEHL | 240 |
| HAYVRAKLMNAYPSYISPIGCLPAHLLGDMWGRFWTNLYSLTVPFGQKPNIDVTDAMVDQ | 300 |
| AWDAQRIFKEAEKFFVSVGLPNMTQGFWENSMLTDPGNVQKAVCSPTAWDLGKGDFRILM | 360 |
| CTKVTMDDFLTAHHEMGHIQYDMAYAAQPFLLRNGANEGFHEAVGEIMSLSAATPKHLKS | 420 |
| IGLLSPDFQEDNETEINFLLKQALTIVGTLPFTYMLEKWRWMVFKGEIPKDQWMKKWWEM | 480 |
| KREIVGVVEPVPHDETYCDPASLFHVSNDYSFIRYYTRTLYQFQFQEALCQAAKHEGPLH | 540 |
| KCDISNSTEAGQKLFNMLRLGKSEPWTLALENVVGAKNMNVRPLLNYFEPLFTWLKDQNK | 660 |
| NSFVGWSTDWSPYAD | 615 |

RATLSCRASQSLYNNFLA (SEQ ID NO: A1)
AASNRAT (SEQ ID NO: A2)
QQYGSSPGYT (SEQ ID NO: A3)
GGTFSSYA (SEQ ID NO: A4)
IIPIFGTA (SEQ ID NO: A5)
ARTLRQYYYYGMDV (SEQ ID NO: A6)
ELVLTQSPGTLSLSPGERATLSCRASQSLYNNFLAWYQQKPGQAPRLLIYAASNRAT
GIPDRFSGSGSGTDFTLTISRLEPEDFAVYYCQQYGSSPGYTFGQGTKLEIKGGSSRSS
QVQLVQSGAEVKKPGSSVKVSCKASGGTFSSYAISWVRQAPGQGLEWMGGIIPIFGT
ANYAQKFQGRVTITADESTSTAYMELSSLRSEDTAVYYCARTLRQYYYYGMDVWG
QGTTVTVSSGSASAPTL (SEQ ID NO: A15)
GGNNIGSKSVH (SEQ ID NO: A16)
DDSDRPS (SEQ ID NO: A17)
QVWDNTLL (SEQ ID NO: A18)
GFTFSSYA (SEQ ID NO: A19)
ISSNGGGT (SEQ ID NO: A20)
VKDDSGSYFSLFDH (SEQ ID NO: A21)
ELMLTQPPSVSVAPGQTARITCGGNNIGSKSVHWYQQKPGQAPVLVVYDDSDRPSGI
PERFSGSNSGNTATLTISRVEAGDEADYYCQVWDNTLLFGGGTELTVLGGGSSRSSG
VQLVESGGGLVQPGGSLRLSCSASGFTFSSYAMHWVRQAPGKGLEYVSGISSNGGG
THYADSVKGRFSISRDNSKNTLYLQMSSLRAEDTAVYYCVKDDSGSYFSLFDHWGQ
GTLVTVSSASTKGPSV (SEQ ID NO: A30)
RASQSISSYLN (SEQ ID NO: A31)
AASSLQS (SEQ ID NO: A32)
QQANSFPFT (SEQ ID NO: A33)
GYTFTSYG (SEQ ID NO: A34)
VNPNGGHT (SEQ ID NO: A35)
ARGKGYYYGMDV (SEQ ID NO: A36)
ELQMTQSPSSLSASVGDRVTITCRASQSISSYLNWYQQKPGKAPKLLIYAASSLQSGV
PSRFSGSGSGTDFTLTISSLQPEDFATYYCQQANSFPFTFGPGTKVDIKGGSSRSSQVQ
LVQSGAEVKKPGASVKVSCKASGYTFTSYGISWVRQAPGQGLEWMGWVNPNGGH
TGYAQKFQGRVTMTRHTATNTVYMELSSLTSDDTAVYYCARGKGYYYGMDVWG
QGTTVLVSSASTKGPSV (SEQ ID NO: A45)
RASQSISSYLN (SEQ ID NO: A46)
AASSLQS (SEQ ID NO: A47)
QQANSFPLT (SEQ ID NO: A48)
GGTLSEN (SEQ ID NO: A49)
IVPMFDIS (SEQ ID NO: A50)
ARDAVATSPGVYQHYGMDV (SEQ ID NO: A51)
ELQMTQSPSSLSASVGDRVTITCRASQSISSYLNWYQQKPGKAPKLLIYAASSLQSGV
PSRFSGSGSGTDFTLTISSLQPEDFATYYCQQANSFPLTFGGGTKVEIKGGSSRSSQVQ
LVQSGAEVKKPGSSVKVSCKASGGTLSENGFSWVRQAPGQGLEWMGRIVPMFDISN
YAERFQDRVTITADGSTNTAYMELRSLRLEDTAVYYCARDAVATSPGVYQHYGMD
VWGQGTTVTVSSASTKGPSV (SEQ ID NO: A60)
RASQSISSYLN (SEQ ID NO: A61)
AASSLQS (SEQ ID NO: A62)
QQSYSTPLT (SEQ ID NO: A63)
GYTFTSYG (SEQ ID NO: A64)
ISAYNGNT (SEQ ID NO: A65)
ARARGLGWGSDY (SEQ ID NO: A66)
ELVMTQSPSSLSASVGDRVTITCRASQSISSYLNWYQQKPGKAPKLLIYAAS
SLQSGVPSRFSGSGSGTDFTLTISSLQPEDFATYYCQQSYSTPLTFGPGTKVDIKGGSS
RSSEVQLVQSGAEVKKPGASVKVSCKASGYTFTSYGISWVRQAPGQGLEWMGWISA
YNGNTNYAQKLQGRVTMTTDTSTSTAYMELRSLRSDDTAVYYCARARGLGWGSD
YWGQGTLVTVSSGSASAPTL (SEQ ID NO: A75)
RASQAIGSWLA (SEQ ID NO: A76)
SASSLQS (SEQ ID NO: A77)
QQLDSYPVT (SEQ ID NO: A78)
GLTVSSNY (SEQ ID NO: A79)
IYSGGST (SEQ ID NO: A80)
ARAIDSGAFDI (SEQ ID NO: A81)
ELVMTQSPSSVSASVGDRVTITCRASQAIGSWLAWYQQKPGKAPKLLISSASSLQSG
VPSRFSGSGSGTDFTLTISSLQPEDFATYYCQQLDSYPVTFGPGTKVDIKGGSSRSSEV
QLLESGGGLIQPGGSLRLSCAASGLTVSSNYMSWVRQAPGKGLEWVSVIYSGGSTY
YADSVKGRFTISRDNSKNTLYLQMNSLRAEDTAVYYCARAIDSGAFDIWGQGTMVT
VSSGSASAPTL (SEQ ID NO: A90)
ELVLTQSPGTLSLSPGERATLSCRASQSLYNNFLAWYQQKPGQAPRLLIYAASNRAT
GIPDRFSGSGSGTDFTLTISRLEPEDFAVYYCQQYGSSPGYTFGQGTKLEIK (SEQ ID
NO: A91)
QVQLVQSGAEVKKPGSSVKVSCKASGGTFSSYAISWVRQAPGQGLEWMGGIIPIFGT
ANYAQKFQGRVTITADESTSTAYMELSSLRSEDTAVYYCARTLRQYYYYGMDVWG
QGTTVTVSSGSASAPTL (SEQ ID NO: A92)
ELMLTQPPSVSVAPGQTARITCGGNNIGSKSVHWYQQKPGQAPVLVVYDDSDRPSGI
PERFSGSNSGNTATLTISRVEAGDEADYYCQVWDNTLLFGGGTELTVLG (SEQ ID
NO: A93)
GVQLVESGGGLVQPGGSLRLSCSASGFTFSSYAMHWVRQAPGKGLEYVSGISSNGG
GTHYADSVKGRFSISRDNSKNTLYLQMSSLRAEDTAVYYCVKDDSGSYFSLFDHWG
QGTLVTVSSASTKGPSV (SEQ ID NO: A94)
ELQMTQSPSSLSASVGDRVTITCRASQSISSYLNWYQQKPGKAPKLLIYAASSLQSGV
PSRFSGSGSGTDFTLTISSLQPEDFATYYCQQANSFPFTFGPGTKVDIK (SEQ ID
NO: A95)
QVQLVQSGAEVKKPGASVKVSCKASGYTFTSYGISWVRQAPGQGLEWMGWVNPN
GGHTGYAQKFQGRVTMTRHTATNTVYMELSSLTSDDTAVYYCARGKGYYYGMDV
WGQGTTVLVSSASTKGPSV (SEQ ID NO: A96)
ELQMTQSPSSLSASVGDRVTITCRASQSISSYLNWYQQKPGKAPKLLIYAASSLQSGV
PSRFSGSGSGTDFTLTISSLQPEDFATYYCQQANSFPLTFGGGTKVEIK (SEQ ID
NO: A97)
QVQLVQSGAEVKKPGSSVKVSCKASGGTLSENGFSWVRQAPGQGLEWMGRIVPMF
DISNYAERFQDRVTITADGSTNTAYMELRSLRLEDTAVYYCARDAVATSPGVYQHY
GMDVWGQGTTVTVSSASTKGPSV (SEQ ID NO: A98)
ELVMTQSPSSLSASVGDRVTITCRASQSISSYLNWYQQKPGKAPKLLIYAASSLQSGV
PSRFSGSGSGTDFTLTISSLQPEDFATYYCQQSYSTPLTFGPGTKVDIK (SEQ ID
NO: A99)
EVQLVQSGAEVKKPGASVKVSCKASGYTFTSYGISWVRQAPGQGLEWMGWISAYN
GNTNYAQKLQGRVTMTTDTSTSTAYMELRSLRSDDTAVYYCARARGLGWGSDYW
GQGTLVTVSSGSASAPTL (SEQ ID NO: A100)
ELVMTQSPSSVSASVGDRVTITCRASQAIGSWLAWYQQKPGKAPKLLISSASSLQSG
VPSRFSGSGSGTDFTLTISSLQPEDFATYYCQQLDSYPVTFGPGTKVDIK (SEQ ID
NO: A101)
EVQLLESGGGLIQPGGSLRLSCAASGLTVSSNYMSWVRQAPGKGLEWVSVIYSGGST
YYADSVKGRFTISRDNSKNTLYLQMNSLRAEDTAVYYCARAIDSGAFDIWGQGTMV
TVSSGSASAPTL (SEQ ID NO: A102)
GAGCTCGTGTTGACACAGTCTCCAGGCACCCTGTCTTTGTCTCCAGGGGAAAGAG
CCACCCTCTCCTGCAGGGCCAGTCAGAGTCTTTACAACAATTTCTTAGCCTGGTA
CCAGCAGAAACCTGGCCAGGCTCCCAGGCTCCTAATCTATGCTGCATCCAACAG
GGCCACTGGCATCCCAGACAGGTTCAGTGGCAGTGGGTCTGGGACAGACTTCAC
TCTCACCATCAGCAGACTGGAGCCTGAAGATTTTGCAGTGTATTACTGTCAGCAG
TATGGTAGCTCACCTGGGTACACTTTTGGCCAGGGGACCAAGCTGGAGATCAAA
GGTGGTTCCTCTAGATCTTCCCAGGTGCAGCTGGTGCAGTCTGGGGCTGAGGTGA
AGAAGCCTGGGTCCTCGGTGAAGGTCTCCTGCAAGGCTTCTGGAGGCACCTTCAG
CAGCTATGCTATCAGCTGGGTGCGACAGGCCCCTGGACAAGGGCTTGAGTGGAT
GGGAGGGATCATCCCTATCTTTGGTACAGCAAACTACGCACAGAAGTTCCAGGG
CAGAGTCACGATTACCGCGGACGAATCCACGAGCACAGCCTACATGGAGCTGAG
CAGCCTGAGATCTGAGGACACGGCCGTGTATTACTGTGCGAGAACCCTTAGACA
ATACTACTACTACGGTATGGACGTCTGGGGCCAAGGGACCACGGTCACCGTCTCC
TCAGGGAGTGCATCCGCCCCAACCCTC (SEQ ID NO: A103)
GAGCTCATGCTGACTCAGCCACCCTCGGTGTCAGTGGCCCCAGGACAGACGGCC
AGGATTACCTGTGGGGGAAACAACATTGGAAGTAAAAGTGTGCACTGGTACCAG
CAGAAGCCAGGCCAGGCCCCTGTGCTGGTCGTCTATGATGATAGCGACCGGCCC
TCAGGGATCCCTGAGCGATTCTCTGGCTCCAACTCTGGGAACACGGCCACCCTGA
CCATCAGCAGGGTCGAAGCCGGGGATGAGGCCGACTATTACTGTCAGGTGTGGG
ATAACACCTTATTATTCGGCGGAGGCACCGAGCTGACCGTCCTCGGTGGTGGTTC
CTCTAGATCTTCCGGGGTGCAGCTGGTGGAGTCTGGGGGAGGCTTGGTCCAGCCT
GGGGGGTCCCTGAGACTCTCCTGTTCAGCCTCTGGATTTACCTTCAGTAGCTATG
CCATGCACTGGGTCCGCCAGGCTCCAGGGAAGGGACTGGAATACGTTTCAGGTA
TTAGTAGTAATGGGGGTGGCACACACTATGCAGACTCCGTGAAGGGCAGATTCA
GCATCTCCAGAGACAATTCCAAGAACACGCTGTATCTTCAAATGAGCAGTCTGA
GAGCTGAGGACACGGCTGTGTATTACTGTGTGAAAGATGATAGTGGGAGCTACT
TCTCCCTATTTGACCACTGGGGCCAGGGAACCCTGGTCACCGTCTCCTCAGCCTC
CACCAAGGGCCCATCGGTC (SEQ ID NO: A104)
GAGCTCCAGATGACCCAGTCTCCATCCTCCCTGTCTGCATCTGTAGGAGACAGAG
TCACCATCACTTGCCGGGCAAGTCAGAGCATTAGCAGCTATTTAAATTGGTATCA
GCAGAAACCAGGGAAAGCCCCTAAGCTCCTGATCTATGCTGCATCCAGTTTGCA
AAGTGGGGTCCCATCAAGGTTCAGTGGCAGTGGATCTGGGACAGATTTCACTCTC
ACCATCAGCAGCCTGCAGCCTGAAGATTTTGCAACTTACTATTGTCAACAGGCTA
ACAGTTTCCCCTTCACTTTCGGCCCTGGGACCAAAGTGGATATCAAAGGTGGTTC
CTCTAGATCTTCCCAGGTGCAGCTGGTGCAGTCTGGGGCTGAGGTGAAGAAGCCT
GGGGCCTCAGTGAAGGTCTCCTGCAAGGCTTCTGGTTACACCTTTACCAGCTATG
GTATCAGCTGGGTGCGACAGGCCCCTGGACAAGGGCTTGAGTGGATGGGATGGG
TGAACCCCAATGGTGGTCACACCGGCTATGCACAGAAGTTCCAGGGCAGGGTCA
CCATGACCCGGCACACCGCCACCAACACAGTCTACATGGAACTGAGTAGCCTGA
CATCTGACGACACGGCCGTCTATTACTGTGCGCGGGGAAAGGGCTACTACTACG
GGATGGACGTCTGGGGCCAAGGGACCACGGTCCTCGTCTCGTCAGCTTCCACCA
AGGGCCCATCGGTC (SEQ ID NO: A105)
GAGCTCCAGATGACCCAGTCTCCATCCTCCCTGTCTGCATCTGTAGGAGACAGAG
TCACCATCACTTGCCGGGCAAGTCAGAGCATTAGCAGCTATTTAAATTGGTATCA
GCAGAAACCAGGGAAAGCCCCTAAGCTCCTGATCTATGCTGCATCCAGTTTGCA
AAGTGGGGTCCCATCAAGGTTCAGTGGCAGTGGATCTGGGACAGATTTCACTCTC
ACCATCAGCAGTCTGCAACCTGAAGATTTTGCAACTTACTATTGTCAACAGGCTA
ACAGTTTCCCTCTCACTTTCGGCGGAGGGACCAAGGTGGAAATCAAAGGTGGTTC
CTCTAGATCTTCCCAGGTGCAGCTGGTGCAGTCTGGGGCTGAGGTGAAGAAGCCT
GGGTCCTCGGTGAAGGTCTCCTGCAAGGCTTCTGGAGGCACCCTCAGTGAAAAT
GGTTTCAGCTGGGTGCGACAGGCCCCTGGACAAGGACTTGAGTGGATGGGCCGA
ATCGTCCCTATGTTTGACATATCAAACTACGCAGAGAGGTTCCAAGACAGAGTCA
CGATTACCGCGGACGGGTCCACGAACACAGCCTACATGGAGCTGCGCAGTTTGA
GATTGGAGGACACGGCCGTATATTACTGTGCGAGAGACGCGGTGGCTACTTCCC
CGGGGGTTTATCAGCACTACGGTATGGACGTCTGGGGCCAAGGGACCACGGTCA
CCGTCTCCTCAGCCTCCACCAAGGGCCCATCGGTC (SEQ ID NO: A106)
GAGCTCGTGATGACTCAGTCTCCATCCTCCCTGTCTGCATCTGTAGGAGACAGAG
TCACCATCACTTGCCGGGCAAGTCAGAGCATTAGCAGCTATTTAAATTGGTATCA
GCAGAAACCAGGGAAAGCCCCTAAGCTCCTGATCTATGCTGCATCCAGTTTGCA
AAGTGGGGTCCCATCAAGGTTCAGTGGCAGTGGATCTGGGACAGATTTCACTCTC
ACCATCAGCAGTCTGCAACCTGAAGATTTTGCAACTTACTACTGTCAACAGAGTT
ACAGTACCCCCCTCACTTTCGGCCCTGGGACCAAAGTGGATATCAAAGGTGGTTC
CTCTAGATCTTCCGAGGTGCAGCTGGTGCAGTCTGGAGCTGAGGTGAAGAAGCC
TGGGGCCTCAGTGAAGGTCTCCTGCAAGGCTTCTGGTTACACCTTTACCAGCTAT
GGTATCAGCTGGGTGCGACAGGCCCCTGGACAAGGGCTTGAGTGGATGGGATGG
ATCAGCGCTTACAATGGTAACACAAACTATGCACAGAAGCTCCAGGGCAGAGTC
ACCATGACCACAGACACATCCACGAGCACAGCCTACATGGAGCTGAGGAGCCTG
AGATCTGACGACACGGCCGTGTATTACTGTGCGAGAGCCAGGGGACTGGGGTGG
GGGAGTGACTACTGGGGCCAGGGAACCCTGGTCACCGTCTCCTCAGGGAGTGCA
TCCGCCCCAACCCTC (SEQ ID NO: A107)
GAGCTCGTGATGACTCAGTCTCCATCTTCCGTGTCTGCATCTGTAGGAGACAGAG
TCACCATCACTTGTCGGGCGAGTCAGGCTATTGGCAGCTGGTTAGCCTGGTATCA
GCAGAAACCAGGGAAAGCCCCCAAGCTCCTCATCTCTTCTGCATCCAGTTTGCAA
AGTGGGGTCCCATCAAGGTTTAGCGGCAGTGGATCTGGGACAGATTTCACTCTCA
CCATCAGCAGCCTGCAGCCTGAAGATTTTGCAACTTATTACTGTCAACAGCTTGA
TAGTTACCCTGTCACTTTCGGCCCTGGGACCAAAGTGGATATCAAAGGTGGTTCC
TCTAGATCTTCCGAGGTGCAGCTGTTGGAGTCTGGAGGAGGCTTGATCCAGCCTG
GGGGGTCCCTGAGACTCTCCTGTGCAGCCTCTGGGTTAACCGTCAGTAGCAACTA
CATGAGTTGGGTCCGCCAGGCTCCAGGGAAGGGGCTGGAGTGGGTCTCAGTTAT
TTATAGCGGTGGTAGCACATACTACGCAGACTCCGTGAAGGGCCGATTCACCATC
TCCAGAGACAATTCCAAGAACACGCTGTATCTTCAAATGAACAGCCTGAGAGCC
GAGGACACGGCCGTGTATTACTGTGCGAGAGCCATTGACTCCGGTGCTTTTGATA
TCTGGGGCCAAGGGACAATGGTCACCGTCTCTTCAGGGAGTGCATCCGCCCCAA
CCCTC (SEQ ID NO: A108)

TABLE 4
Table of identification of SEQ ID NOs.

| Antibody number: | 39 | 37 | 27 | 38 | 24 | 36 |
|---|---|---|---|---|---|---|
| CDR L1 | SEQ ID NO: 1 | SEQ ID NO: 16 | SEQ ID NO: 31 | SEQ ID NO: 46 | SEQ ID NO: 61 | SEQ ID NO: 76 |
| CDR L2 | SEQ ID NO: 2 | SEQ ID NO: 17 | SEQ ID NO: 32 | SEQ ID NO: 47 | SEQ ID NO: 62 | SEQ ID NO: 77 |
| CDR L3 | SEQ ID NO: 3 | SEQ ID NO: 18 | SEQ ID NO: 33 | SEQ ID NO: 48 | SEQ ID NO: 63 | SEQ ID NO: 78 |
| CDR H1 | SEQ ID NO: 4 | SEQ ID NO: 19 | SEQ ID NO: 34 | SEQ ID NO: 49 | SEQ ID NO: 64 | SEQ ID NO: 79 |
| CDR H2 | SEQ ID NO: 5 | SEQ ID NO: 20 | SEQ ID NO: 35 | SEQ ID NO: 50 | SEQ ID NO: 65 | SEQ ID NO: 80 |
| CDR H3 | SEQ ID NO: 6 | SEQ ID NO: 21 | SEQ ID NO: 36 | SEQ ID NO: 51 | SEQ ID NO: 66 | SEQ ID NO: 81 |
| FR L1 | SEQ ID NO: 7 | SEQ ID NO: 22 | SEQ ID NO: 37 | SEQ ID NO: 52 | SEQ ID NO: 67 | SEQ ID NO: 82 |
| FR L2 | SEQ ID NO: 8 | SEQ ID NO: 23 | SEQ ID NO: 38 | SEQ ID NO: 53 | SEQ ID NO: 68 | SEQ ID NO: 83 |
| FR L3 | SEQ ID NO: 9 | SEQ ID NO: 24 | SEQ ID NO: 39 | SEQ ID NO: 54 | SEQ ID NO: 69 | SEQ ID NO: 84 |
| FR L4 | SEQ ID NO: 10 | SEQ ID NO: 25 | SEQ ID NO: 40 | SEQ ID NO: 55 | SEQ ID NO: 70 | SEQ ID NO: 85 |
| FR H1 | SEQ ID NO: 11 | SEQ ID NO: 26 | SEQ ID NO: 41 | SEQ ID NO: 56 | SEQ ID NO: 71 | SEQ ID NO: 86 |
| FR H2 | SEQ ID NO: 12 | SEQ ID NO: 27 | SEQ ID NO: 42 | SEQ ID NO: 57 | SEQ ID NO: 72 | SEQ ID NO: 87 |
| FR H3 | SEQ ID NO: 13 | SEQ ID NO: 28 | SEQ ID NO: 43 | SEQ ID NO: 58 | SEQ ID NO: 73 | SEQ ID NO: 88 |
| FR H4 | SEQ ID NO: 14 | SEQ ID NO: 29 | SEQ ID NO: 44 | SEQ ID NO: 59 | SEQ ID NO: 74 | SEQ ID NO: 89 |
| Full AA sequence | SEQ ID NO: 15 | SEQ ID NO: 30 | SEQ ID NO: 45 | SEQ ID NO: 60 | SEQ ID NO: 75 | SEQ ID NO: 90 |
| VL | SEQ ID NO: 91 | SEQ ID NO: 93 | SEQ ID NO: 95 | SEQ ID NO: 97 | SEQ ID NO: 99 | SEQ ID NO: 101 |
| VH | SEQ ID NO: 92 | SEQ ID NO: 94 | SEQ ID NO: 96 | SEQ ID NO: 98 | SEQ ID NO: 100 | SEQ ID NO: 102 |
| Full DNA seq | SEQ ID NO: 103 | SEQ ID NO: 104 | SEQ ID NO: 105 | SEQ ID NO: 106 | SEQ ID NO: 107 | SEQ ID NO: 108 |

NOTE:
each of the identifiers in the above Table 4 are preceded with an A; i.e., SEQ ID NO: 1 is actually SEQ ID NO: A1 as shown in the above partial informal sequence listing. For example, SEQ ID NO: 2 is SEQ ID NO: A2, SEQ ID NO: 3 is SEQ ID NO: A3, and so on through to SEQ ID NO: 108 is SEQ ID NO: A108.

### OTHER EMBODIMENTS

It is to be understood that while the invention has been described in conjunction with the detailed description thereof, the foregoing description is intended to illustrate and not limit the scope of the invention.

What is claimed is:

1. A nucleic acid molecule comprising a nucleotide sequence encoding a chimeric antigen receptor (CAR), wherein the chimeric antigen receptor comprises: an scFv targeting SAR-CoV2 Spike Protein, a spacer, a transmembrane domain, a co-stimulatory domain, and a CD3ζ signaling domain.

2. The nucleic acid molecule of claim 1, wherein the scFv comprises the amino acid sequence of any of SEQ ID NOs:1, 41-45 or variant thereof having 1-5 amino acid modifications.

3. The nucleic acid molecule of claim 1, wherein the scFv comprises the heavy and light chain CDRs of any amino acid sequence of SEQ ID NOs:1, 41-45.

4. The nucleic acid molecule of claim 1, wherein the transmembrane domain is selected from: a CD4 transmembrane domain or variant thereof having 1-5 amino acid modifications, a CD8 transmembrane domain or variant thereof having 1-5 amino acid modifications, a CD28 transmembrane domain or a variant thereof having 1-5 amino acid modifications.

5. The nucleic acid molecule of claim 1, wherein the transmembrane domain is a CD28 transmembrane domain.

6. The nucleic acid molecule of claim 1, wherein the costimulatory domain is a CD28, 4-1BB, or a 2B4.

7. The nucleic acid molecule of claim 1, wherein the costimulatory comprises the amino acid sequence of any SEQ ID NO:22-25 or a variant thereof having 1-5 amino acid modifications.

8. The nucleic acid molecule of claim 1, wherein the CD3ζ signaling domain comprises the amino acid sequence of SEQ ID NO:21.

9. The nucleic acid molecule of claim 1, wherein a linker of 3 to 15 amino acids is located between the costimulatory domain and the CD3ζ signaling domain or variant thereof.

10. The nucleic acid molecule of claim 1, wherein the spacer comprises any one of SEQ ID NOs:2-12 and 70 or a variant thereof having 1-5 amino acid modifications.

11. A nucleic acid molecule comprising a nucleotide sequence encoding a chimeric antigen receptor (CAR), wherein the CAR comprises: an extracellular domain of human ACE2 or a variant there of, a spacer, a transmembrane domain, a co-stimulatory domain, and a CD3ζ signaling domain.

12. The nucleic acid molecule of claim 11, wherein the variant human ACE2 extracellular domain comprises the amino acid sequence of any of SEQ ID NOs: 29, 30, 31, 32, 33, 38, 39, and 40, or variant thereof having 1-5 amino acid modifications.

13. The nucleic acid molecule of claim 11, wherein the variant human ACE2 consists of the amino acid sequence of any of SEQ ID NOs: 29, 30, 31, 32, 33, 38, 39, and 40; amino acids 18-740 of any of SEQ ID NOs: 29, 31, 32, and 33; and amino acids 18-615 of any of SEQ I NOs: 30, 38, 39 and 40.

14. The nucleic acid molecule of claim 1, wherein the transmembrane domain is selected from: a CD4 transmembrane domain or variant thereof having 1-5 amino acid modifications, a CD8 transmembrane domain or variant thereof having 1-5 amino acid modifications, a CD28 transmembrane domain or a variant thereof having 1-5 amino acid modifications.

15. The nucleic acid molecule of claim 11, wherein the transmembrane domain is a CD28 transmembrane domain.

16. The nucleic acid molecule of claim 11, wherein the costimulatory domain is a CD28, 4-1BB, or a 2B4.

17. The nucleic acid molecule of claim 11, wherein the costimulatory comprises the amino acid sequence of any if SEQ ID NO:22-25 or a variant thereof having 1-5 amino acid modifications.

18. The nucleic acid molecule of claim 11, wherein the CD3ζ signaling domain comprises the amino acid sequence of SEQ ID NO:21.

19. The nucleic acid molecule of claim 11, wherein a linker of 3 to 15 amino acids is located between the costimulatory domain and the CD3ζ signaling domain or variant thereof.

20. The nucleic acid molecule of claim 11, wherein the spacer comprises any one of SEQ ID NOs:2-12 and 70 or a variant thereof having 1-5 amino acid modifications.

21. An expression vector comprising the nucleic acid molecule of any of claims 1-20.

22. The expression vector of claim 21 further comprising a sequence encoding human IL-15 or a soluble fragment thereof.

23. The expression vector of claim 21 or claim 22, wherein the vector is a viral vector.

24. A population of human NK cells transduced by a vector comprising the nucleic acid molecule of any of claims 1-20.

25. A population of human NK cells transduced by the vector of any of claims 21-23.

26. A method of treating a patient infected with SARS-CoV-2 comprising administering a composition comprising the population of human NK cells of claim 24 or claim 25 wherein the cells are autologous or allogenic to the patient.

27. A method of reducing or eliminating SARS-CoV-2 in a subject comprising administering a population of autologous or allogeneic NK cells transduced by a vector comprising the nucleic acid molecule of any of claims 1-20.


SEQUENCE LISTING (162 sequences)

SEQ ID NO: 1 - protein, 248 aa, Artificial Sequence
  source: /note=“Description of Artificial Sequence Synthetic polypeptide”
ELQMTQSPSSLSASVGDRVTITCRASQSISSYLNWYQQKPGKAPKLLIYAASSLQSGVPSRFSGSGSGTDFTLTISSLQPEDFATYYCQQANSFPLTFGGGTKVEIKGGSSRSSQVQLVQSGAEVKKPGSSVKVSCKASGGTLSENGFSWVRQAPGQGLEWMGRIVPMFDISNYAERFQDRVTITADGSTNTAYMELRSLRLEDTAVYYCARDAVATSPGVYQHYGMDVWGQGTTVTVSSASTKGPSV

SEQ ID NO: 2 - protein, 10 aa, Artificial Sequence
  source: /note=“Description of Artificial Sequence Synthetic peptide”
GGGSSGGGSG

SEQ ID NO: 3 - protein, 12 aa, Artificial Sequence
  source: /note=“Description of Artificial Sequence Synthetic peptide”
ESKYGPPCPPCP

SEQ ID NO: 4 - protein, 12 aa, Artificial Sequence
  source: /note=“Description of Artificial Sequence Synthetic peptide”
ESKYGPPCPSCP

SEQ ID NO: 5 - protein, 22 aa, Artificial Sequence
  source: /note=“Description of Artificial Sequence Synthetic peptide”
ESKYGPPCPPCPGGGSSGGGSG

SEQ ID NO: 6 - protein, 39 aa, Artificial Sequence
  source: /note=“Description of Artificial Sequence Synthetic polypeptide”
IEVMYPPPYLDNEKSNGTIIHVKGKHLCPSPLFPGPSKP

SEQ ID NO: 7 - protein, 48 aa, Artificial Sequence
  source: /note=“Description of Artificial Sequence Synthetic polypeptide”
AKPTTTPAPRPPTPAPTIASQPLSLRPEACRPAAGGAVHTRGLDFACD

SEQ ID NO: 8 - protein, 45 aa, Artificial Sequence
  source: /note=“Description of Artificial Sequence Synthetic polypeptide”
TTTPAPRPPTPAPTIASQPLSLRPEACRPAAGGAVHTRGLDFACD

SEQ ID NO: 9 - protein, 129 aa, Artificial Sequence
  source: /note=“Description of Artificial Sequence Synthetic polypeptide”
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 229 aa, Artificial Sequence
  source: /note=“Description of Artificial Sequence Synthetic polypeptide”
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, Artificial Sequence
  source: /note=“Description of Artificial Sequence Synthetic polypeptide”
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, Artificial Sequence
  source: /note=“Description of Artificial Sequence Synthetic polypeptide”
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 21 aa, Homo sapiens
LCYLLDGILFIYGVILTALFL

SEQ ID NO: 14 - protein, 27 aa, Homo sapiens
FWVLVVVGGVLACYSLLVTVAFIIFWV

SEQ ID NO: 15 - protein, 28 aa, Homo sapiens
MFWVLVVVGGVLACYSLLVTVAFIIFWV

SEQ ID NO: 16 - protein, 22 aa, Homo sapiens
MALIVLGGVAGLLLFIGLGIFF

SEQ ID NO: 17 - protein, 21 aa, Homo sapiens
IYIWAPLAGTCGVLLLSLVIT

SEQ ID NO: 18 - protein, 23 aa, Homo sapiens
IYIWAPLAGTCGVLLLSLVITLY

SEQ ID NO: 19 - protein, 24 aa, Homo sapiens
IYIWAPLAGTCGVLLLSLVITLYC

SEQ ID NO: 20 - protein, 27 aa, Homo sapiens
IISFFLALTSTALLFLLFFLTLRFSVV

SEQ ID NO: 21 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 41 aa, Homo sapiens
RSKRSRLLHSDYMNMTPRRPGPTRKHYQPYAPPRDFAAYRS

SEQ ID NO: 23 - protein, 41 aa, Homo sapiens
RSKRSRGGHSDYMNMTPRRPGPTRKHYQPYAPPRDFAAYRS

SEQ ID NO: 24 - protein, 42 aa, Homo sapiens
KRGRKKLLYIFKQPFMRPVQTTQEEDGCSCRFPEEEEGGCEL

SEQ ID NO: 25 - protein, 42 aa, Unknown
  source: /note=“Description of Unknown OX40 sequence”
ALYLLRRDQRLPPDAHKPPGGGSFRTPIQEEQADAHSTLAKI

SEQ ID NO: 26 - protein, 323 aa, Artificial Sequence
  source: /note=“Description of Artificial Sequence Synthetic polypeptide”
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 27 - protein, 24 aa, Artificial Sequence
  source: /note=“Description of Artificial Sequence Synthetic peptide”
LEGGGEGRGSLLTCGDVEENPGPR

SEQ ID NO: 28 - protein, 354 aa, Artificial Sequence
  source: /note=“Description of Artificial Sequence Synthetic polypeptide”
LVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 29 - protein, 740 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 30 - protein, 615 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 31 - protein, 740 aa, Artificial Sequence
  source: /note=“Description of Artificial Sequence Synthetic polypeptide”
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGSFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 32 - protein, 740 aa, Artificial Sequence
  source: /note=“Description of Artificial Sequence Synthetic polypeptide”
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCFPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 33 - protein, 740 aa, Artificial Sequence
  source: /note=“Description of Artificial Sequence Synthetic polypeptide”
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCSPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 34 - protein, 4 aa, Artificial Sequence
  source: /note=“Description of Artificial Sequence Synthetic peptide”
GGGS

SEQ ID NO: 35 - protein, 5 aa, Artificial Sequence
  source: /note=“Description of Artificial Sequence Synthetic peptide”
GGGGS

SEQ ID NO: 36 - protein, 22 aa, Homo sapiens
MLLLVTSLLLCELPHPAFLLIP

SEQ ID NO: 37 - protein, 7 aa, Artificial Sequence
  source: /note=“Description of Artificial Sequence Synthetic peptide”
GGSSRSS

SEQ ID NO: 38 - protein, 615 aa, Artificial Sequence
  source: /note=“Description of Artificial Sequence Synthetic polypeptide”
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGSFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 39 - protein, 615 aa, Artificial Sequence
  source: /note=“Description of Artificial Sequence Synthetic polypeptide”
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCFPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 40 - protein, 615 aa, Artificial Sequence
  source: /note=“Description of Artificial Sequence Synthetic polypeptide”
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCSPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 41 - protein, 241 aa, Artificial Sequence
  source: /note=“Description of Artificial Sequence Synthetic polypeptide”
ELVMTQSPSSLSASVGDRVTITCRASQSISSYLNWYQQKPGKAPKLLIYAASSLQSGVPSRFSGSGSGTDFTLTISSLQPEDFATYYCQQSYSTPLTFGPGTKVDIKGGSSRSSEVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARARGLGWGSDYWGQGTLVTVSSGSASAPTL

SEQ ID NO: 42 - protein, 239 aa, Artificial Sequence
  source: /note=“Description of Artificial Sequence Synthetic polypeptide”
ELVMTQSPSSVSASVGDRVTITCRASQAIGSWLAWYQQKPGKAPKLLISSASSLQSGVPSRFSGSGSGTDFTLTISSLQPEDFATYYCQQLDSYPVTFGPGTKVDIKGGSSRSSEVQLLESGGGLIQPGGSLRLSCAASGLTVSSNYMSWVRQAPGKGLEWVSVIYSGGSTYYADSVKGRFTISRDNSKNTLYLQMNSLRAEDTAVYYCARAIDSGAFDIWGQGTMVTVSSGSASAPTL

SEQ ID NO: 43 - protein, 245 aa, Artificial Sequence
  source: /note=“Description of Artificial Sequence Synthetic polypeptide”
ELVLTQSPGTLSLSPGERATLSCRASQSLYNNFLAWYQQKPGQAPRLLIYAASNRATGIPDRFSGSGSGTDFTLTISRLEPEDFAVYYCQQYGSSPGYTFGQGTKLEIKGGSSRSSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTLRQYYYYGMDVWGQGTTVTVSSGSASAPTL

SEQ ID NO: 44 - protein, 242 aa, Artificial Sequence
  source: /note=“Description of Artificial Sequence Synthetic polypeptide”
ELMLTQPPSVSVAPGQTARITCGGNNIGSKSVHWYQQKPGQAPVLVVYDDSDRPSGIPERFSGSNSGNTATLTISRVEAGDEADYYCQVWDNTLLFGGGTELTVLGGGSSRSSGVQLVESGGGLVQPGGSLRLSCSASGFTFSSYAMHWVRQAPGKGLEYVSGISSNGGGTHYADSVKGRFSISRDNSKNTLYLQMSSLRAEDTAVYYCVKDDSGSYFSLFDHWGQGTLVTVSSASTKGPSV

SEQ ID NO: 45 - protein, 241 aa, Artificial Sequence
  source: /note=“Description of Artificial Sequence Synthetic polypeptide”
ELQMTQSPSSLSASVGDRVTITCRASQSISSYLNWYQQKPGKAPKLLIYAASSLQSGVPSRFSGSGSGTDFTLTISSLQPEDFATYYCQQANSFPFTFGPGTKVDIKGGSSRSSQVQLVQSGAEVKKPGASVKVSCKASGYTFTSYGISWVRQAPGQGLEWMGWVNPNGGHTGYAQKFQGRVTMTRHTATNTVYMELSSLTSDDTAVYYCARGKGYYYGMDVWGQGTTVLVSSASTKGPSV

SEQ ID NO: 46 - protein, 20 aa, Homo sapiens
MYRMQLLSCIALSLALVTNS

SEQ ID NO: 47 - protein, 131 aa, Homo sapiens
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIN

SEQ ID NO: 48 - protein, 282 aa, Homo sapiens
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNK

SEQ ID NO: 49 - protein, 936 aa, Artificial Sequence
  source: /note=“Description of Artificial Sequence Synthetic polypeptide”
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 811 aa, Artificial Sequence
  source: /note=“Description of Artificial Sequence Synthetic polypeptide”
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 936 aa, Artificial Sequence
  source: /note=“Description of Artificial Sequence Synthetic polypeptide”
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGSFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 811 aa, Artificial Sequence
  source: /note=“Description of Artificial Sequence Synthetic polypeptide”
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGSFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 936 aa, Artificial Sequence
  source: /note=“Description of Artificial Sequence Synthetic polypeptide”
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCFPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 811 aa, Artificial Sequence
  source: /note=“Description of Artificial Sequence Synthetic polypeptide”
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCFPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 936 aa, Artificial Sequence
  source: /note=“Description of Artificial Sequence Synthetic polypeptide”
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCSPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 811 aa, Artificial Sequence
  source: /note=“Description of Artificial Sequence Synthetic polypeptide”
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCSPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 919 aa, Artificial Sequence
  source: /note=“Description of Artificial Sequence Synthetic polypeptide”
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 794 aa, Artificial Sequence
  source: /note=“Description of Artificial Sequence Synthetic polypeptide”
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 919 aa, Artificial Sequence
  source: /note=“Description of Artificial Sequence Synthetic polypeptide”
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGSFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 794 aa, Artificial Sequence
  source: /note=“Description of Artificial Sequence Synthetic polypeptide”
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGSFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 919 aa, Artificial Sequence
  source: /note=“Description of Artificial Sequence Synthetic polypeptide”
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCFPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 794 aa, Artificial Sequence
  source: /note=“Description of Artificial Sequence Synthetic polypeptide”
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCFPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 919 aa, Artificial Sequence
  source: /note=“Description of Artificial Sequence Synthetic polypeptide”
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCSPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 794 aa, Artificial Sequence
  source: /note=“Description of Artificial Sequence Synthetic polypeptide”
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCSPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 444 aa, Artificial Sequence
  source: /note=“Description of Artificial Sequence Synthetic polypeptide”
ELQMTQSPSSLSASVGDRVTITCRASQSISSYLNWYQQKPGKAPKLLIYAASSLQSGVPSRFSGSGSGTDFTLTISSLQPEDFATYYCQQANSFPLTFGGGTKVEIKGGSSRSSQVQLVQSGAEVKKPGSSVKVSCKASGGTLSENGFSWVRQAPGQGLEWMGRIVPMFDISNYAERFQDRVTITADGSTNTAYMELRSLRLEDTAVYYCARDAVATSPGVYQHYGMDVWGQGTTVTVSSASTKGPSVLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 437 aa, Artificial Sequence
  source: /note=“Description of Artificial Sequence Synthetic polypeptide”
ELVMTQSPSSLSASVGDRVTITCRASQSISSYLNWYQQKPGKAPKLLIYAASSLQSGVPSRFSGSGSGTDFTLTISSLQPEDFATYYCQQSYSTPLTFGPGTKVDIKGGSSRSSEVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARARGLGWGSDYWGQGTLVTVSSGSASAPTLLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 435 aa, Artificial Sequence
  source: /note=“Description of Artificial Sequence Synthetic polypeptide”
ELVMTQSPSSVSASVGDRVTITCRASQAIGSWLAWYQQKPGKAPKLLISSASSLQSGVPSRFSGSGSGTDFTLTISSLQPEDFATYYCQQLDSYPVTFGPGTKVDIKGGSSRSSEVQLLESGGGLIQPGGSLRLSCAASGLTVSSNYMSWVRQAPGKGLEWVSVIYSGGSTYYADSVKGRFTISRDNSKNTLYLQMNSLRAEDTAVYYCARAIDSGAFDIWGQGTMVTVSSGSASAPTLLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 441 aa, Artificial Sequence
  source: /note=“Description of Artificial Sequence Synthetic polypeptide”
ELVLTQSPGTLSLSPGERATLSCRASQSLYNNFLAWYQQKPGQAPRLLIYAASNRATGIPDRFSGSGSGTDFTLTISRLEPEDFAVYYCQQYGSSPGYTFGQGTKLEIKGGSSRSSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTLRQYYYYGMDVWGQGTTVTVSSGSASAPTLLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 438 aa, Artificial Sequence
  source: /note=“Description of Artificial Sequence Synthetic polypeptide”
ELMLTQPPSVSVAPGQTARITCGGNNIGSKSVHWYQQKPGQAPVLVVYDDSDRPSGIPERFSGSNSGNTATLTISRVEAGDEADYYCQVWDNTLLFGGGTELTVLGGGSSRSSGVQLVESGGGLVQPGGSLRLSCSASGFTFSSYAMHWVRQAPGKGLEYVSGISSNGGGTHYADSVKGRFSISRDNSKNTLYLQMSSLRAEDTAVYYCVKDDSGSYFSLFDHWGQGTLVTVSSASTKGPSVLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 437 aa, Artificial Sequence
  source: /note=“Description of Artificial Sequence Synthetic polypeptide”
ELQMTQSPSSLSASVGDRVTITCRASQSISSYLNWYQQKPGKAPKLLIYAASSLQSGVPSRFSGSGSGTDFTLTISSLQPEDFATYYCQQANSFPFTFGPGTKVDIKGGSSRSSQVQLVQSGAEVKKPGASVKVSCKASGYTFTSYGISWVRQAPGQGLEWMGWVNPNGGHTGYAQKFQGRVTMTRHTATNTVYMELSSLTSDDTAVYYCARGKGYYYGMDVWGQGTTVLVSSASTKGPSVLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 16 aa, Artificial Sequence
  source: /note=“Description of Artificial Sequence Synthetic peptide”
LEPKSCDKTHTCPPCP

SEQ ID NO: 72 - DNA, 20 bp, Artificial Sequence
  source: /note=“Description of Artificial Sequence Synthetic primer”
gctgaacatgtcaacaactc

SEQ ID NO: 73 - DNA, 20 bp, Artificial Sequence
  source: /note=“Description of Artificial Sequence Synthetic primer”
gcaatgatggattgactagc

SEQ ID NO: 74 - protein, 6 aa, Artificial Sequence
  source: /note=“Description of Artificial Sequence Synthetic 6xHis tag”
HHHHHH

SEQ ID NO: 75 - DNA, 18 bp, Artificial Sequence
  source: /note=“Description of Artificial Sequence Synthetic oligonucleotide”
catcaccatcaccatcac

SEQ ID NO: 76 - DNA, 2220 bp, Homo sapiens
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttcc

SEQ ID NO: 77 - DNA, 1845 bp, Homo sapiens
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagac

SEQ ID NO: 78 - DNA, 2220 bp, Artificial Sequence
  source: /note=“Description of Artificial Sequence Synthetic polynucleotide”
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagcttttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttcc

SEQ ID NO: 79 - DNA, 2220 bp, Artificial Sequence
  source: /note=“Description of Artificial Sequence Synthetic polynucleotide”
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgctttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttcc

SEQ ID NO: 80 - DNA, 2220 bp, Artificial Sequence
  source: /note=“Description of Artificial Sequence Synthetic polynucleotide”
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgctctcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttcc

SEQ ID NO: 81 - DNA, 693 bp, Artificial Sequence
  source: /note=“Description of Artificial Sequence Synthetic polynucleotide”
gagtccaaatatggtcccccatgcccaccctgcccagcacctgagttcgaagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaagctagc

SEQ ID NO: 82 - protein, 231 aa, Artificial Sequence
  source: /note=“Description of Artificial Sequence Synthetic polypeptide”
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 83 - DNA, 687 bp, Artificial Sequence
  source: /note=“Description of Artificial Sequence Synthetic polynucleotide”
tcttccgacaaaactcacacaagcccaccgtcaccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 84 - protein, 229 aa, Artificial Sequence
  source: /note=“Description of Artificial Sequence Synthetic polypeptide”
SSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - DNA, 1845 bp, Artificial Sequence
  source: /note=“Description of Artificial Sequence Synthetic polynucleotide”
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagcttttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagac

SEQ ID NO: 86 - DNA, 1845 bp, Artificial Sequence
  source: /note=“Description of Artificial Sequence Synthetic polynucleotide”
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgctttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagac

SEQ ID NO: 87 - DNA, 1845 bp, Artificial Sequence
  source: /note=“Description of Artificial Sequence Synthetic polynucleotide”
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgctctcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagac

SEQ ID NO: 88 - protein, 18 aa, Artificial Sequence
  source: /note=“Description of Artificial Sequence Synthetic peptide”
RATLSCRASQSLYNNFLA

SEQ ID NO: 89 - protein, 7 aa, Artificial Sequence
  source: /note=“Description of Artificial Sequence Synthetic peptide”
AASNRAT

SEQ ID NO: 90 - protein, 10 aa, Artificial Sequence
  source: /note=“Description of Artificial Sequence Synthetic peptide”
QQYGSSPGYT

SEQ ID NO: 91 - protein, 8 aa, Artificial Sequence
  source: /note=“Description of Artificial Sequence Synthetic peptide”
GGTFSSYA

SEQ ID NO: 92 - protein, 8 aa, Artificial Sequence
  source: /note=“Description of Artificial Sequence Synthetic peptide”
IIPIFGTA

SEQ ID NO: 93 - protein, 14 aa, Artificial Sequence
  source: /note=“Description of Artificial Sequence Synthetic peptide”
ARTLRQYYYYGMDV

SEQ ID NO: 94 - protein, 11 aa, Artificial Sequence
  source: /note=“Description of Artificial Sequence Synthetic peptide”
GGNNIGSKSVH

SEQ ID NO: 95 - protein, 7 aa, Artificial Sequence
  source: /note=“Description of Artificial Sequence Synthetic peptide”
DDSDRPS

SEQ ID NO: 96 - protein, 8 aa, Artificial Sequence
  source: /note=“Description of Artificial Sequence Synthetic peptide”
QVWDNTLL

SEQ ID NO: 97 - protein, 8 aa, Artificial Sequence
  source: /note=“Description of Artificial Sequence Synthetic peptide”
GFTFSSYA

SEQ ID NO: 98 - protein, 8 aa, Artificial Sequence
  source: /note=“Description of Artificial Sequence Synthetic peptide”
ISSNGGGT

SEQ ID NO: 99 - protein, 14 aa, Artificial Sequence
  source: /note=“Description of Artificial Sequence Synthetic peptide”
VKDDSGSYFSLFDH

SEQ ID NO: 100 - protein, 11 aa, Artificial Sequence
  source: /note=“Description of Artificial Sequence Synthetic peptide”
RASQSISSYLN

SEQ ID NO: 101 - protein, 7 aa, Artificial Sequence
  source: /note=“Description of Artificial Sequence Synthetic peptide”
AASSLQS

SEQ ID NO: 102 - protein, 9 aa, Artificial Sequence
  source: /note=“Description of Artificial Sequence Synthetic peptide”
QQANSFPFT

SEQ ID NO: 103 - protein, 8 aa, Artificial Sequence
  source: /note=“Description of Artificial Sequence Synthetic peptide”
GYTFTSYG

SEQ ID NO: 104 - protein, 8 aa, Artificial Sequence
  source: /note=“Description of Artificial Sequence Synthetic peptide”
VNPNGGHT

SEQ ID NO: 105 - protein, 12 aa, Artificial Sequence
  source: /note=“Description of Artificial Sequence Synthetic peptide”
ARGKGYYYGMDV

SEQ ID NO: 106 - protein, 9 aa, Artificial Sequence
  source: /note=“Description of Artificial Sequence Synthetic peptide”
QQANSFPLT

SEQ ID NO: 107 - protein, 7 aa, Artificial Sequence
  source: /note=“Description of Artificial Sequence Synthetic peptide”
GGTLSEN

SEQ ID NO: 108 - protein, 8 aa, Artificial Sequence
  source: /note=“Description of Artificial Sequence Synthetic peptide”
IVPMFDIS

SEQ ID NO: 109 - protein, 19 aa, Artificial Sequence
  source: /note=“Description of Artificial Sequence Synthetic peptide”
ARDAVATSPGVYQHYGMDV

SEQ ID NO: 110 - protein, 9 aa, Artificial Sequence
  source: /note=“Description of Artificial Sequence Synthetic peptide”
QQSYSTPLT

SEQ ID NO: 111 - protein, 8 aa, Artificial Sequence
  source: /note=“Description of Artificial Sequence Synthetic peptide”
ISAYNGNT

SEQ ID NO: 112 - protein, 12 aa, Artificial Sequence
  source: /note=“Description of Artificial Sequence Synthetic peptide”
ARARGLGWGSDY

SEQ ID NO: 113 - protein, 11 aa, Artificial Sequence
  source: /note=“Description of Artificial Sequence Synthetic peptide”
RASQAIGSWLA

SEQ ID NO: 114 - protein, 7 aa, Artificial Sequence
  source: /note=“Description of Artificial Sequence Synthetic peptide”
SASSLQS

SEQ ID NO: 115 - protein, 9 aa, Artificial Sequence
  source: /note=“Description of Artificial Sequence Synthetic peptide”
QQLDSYPVT

SEQ ID NO: 116 - protein, 8 aa, Artificial Sequence
  source: /note=“Description of Artificial Sequence Synthetic peptide”
GLTVSSNY

SEQ ID NO: 117 - protein, 7 aa, Artificial Sequence
  source: /note=“Description of Artificial Sequence Synthetic peptide”
IYSGGST

SEQ ID NO: 118 - protein, 11 aa, Artificial Sequence
  source: /note=“Description of Artificial Sequence Synthetic peptide”
ARAIDSGAFDI

SEQ ID NO: 119 - protein, 109 aa, Artificial Sequence
  source: /note=“Description of Artificial Sequence Synthetic polypeptide”
ELVLTQSPGTLSLSPGERATLSCRASQSLYNNFLAWYQQKPGQAPRLLIYAASNRATGIPDRFSGSGSGTDFTLTISRLEPEDFAVYYCQQYGSSPGYTFGQGTKLEIK

SEQ ID NO: 120 - protein, 129 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTLRQYYYYGMDVWGQGTTVTVSSGSASAPTL

SEQ ID NO: 121 - protein, 106 aa, Artificial Sequence
  source: /note=“Description of Artificial Sequence Synthetic polypeptide”
ELMLTQPPSVSVAPGQTARITCGGNNIGSKSVHWYQQKPGQAPVLVVYDDSDRPSGIPERFSGSNSGNTATLTISRVEAGDEADYYCQVWDNTLLFGGGTELTVLG

SEQ ID NO: 122 - protein, 129 aa, Artificial Sequence
  source: /note=“Description of Artificial Sequence Synthetic polypeptide”
GVQLVESGGGLVQPGGSLRLSCSASGFTFSSYAMHWVRQAPGKGLEYVSGISSNGGGTHYADSVKGRFSISRDNSKNTLYLQMSSLRAEDTAVYYCVKDDSGSYFSLFDHWGQGTLVTVSSASTKGPSV

SEQ ID NO: 123 - protein, 107 aa, Artificial Sequence
  source: /note=“Description of Artificial Sequence Synthetic polypeptide”
ELQMTQSPSSLSASVGDRVTITCRASQSISSYLNWYQQKPGKAPKLLIYAASSLQSGVPSRFSGSGSGTDFTLTISSLQPEDFATYYCQQANSFPFTFGPGTKVDIK

SEQ ID NO: 124 - protein, 127 aa, Artificial Sequence
  source: /note=“Description of Artificial Sequence Synthetic polypeptide”
QVQLVQSGAEVKKPGASVKVSCKASGYTFTSYGISWVRQAPGQGLEWMGWVNPNGGHTGYAQKFQGRVTMTRHTATNTVYMELSSLTSDDTAVYYCARGKGYYYGMDVWGQGTTVLVSSASTKGPSV

SEQ ID NO: 125 - protein, 107 aa, Artificial Sequence
  source: /note=“Description of Artificial Sequence Synthetic polypeptide”
ELQMTQSPSSLSASVGDRVTITCRASQSISSYLNWYQQKPGKAPKLLIYAASSLQSGVPSRFSGSGSGTDFTLTISSLQPEDFATYYCQQANSFPLTFGGGTKVEIK

SEQ ID NO: 126 - protein, 134 aa, Artificial Sequence
  source: /note=“Description of Artificial Sequence Synthetic polypeptide”
QVQLVQSGAEVKKPGSSVKVSCKASGGTLSENGFSWVRQAPGQGLEWMGRIVPMFDISNYAERFQDRVTITADGSTNTAYMELRSLRLEDTAVYYCARDAVATSPGVYQHYGMDVWGQGTTVTVSSASTKGPSV

SEQ ID NO: 127 - protein, 107 aa, Artificial Sequence
  source: /note=“Description of Artificial Sequence Synthetic polypeptide”
ELVMTQSPSSLSASVGDRVTITCRASQSISSYLNWYQQKPGKAPKLLIYAASSLQSGVPSRFSGSGSGTDFTLTISSLQPEDFATYYCQQSYSTPLTFGPGTKVDIK

SEQ ID NO: 128 - protein, 127 aa, Artificial Sequence
  source: /note=“Description of Artificial Sequence Synthetic polypeptide”
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARARGLGWGSDYWGQGTLVTVSSGSASAPTL

SEQ ID NO: 129 - protein, 107 aa, Artificial Sequence
  source: /note=“Description of Artificial Sequence Synthetic polypeptide”
ELVMTQSPSSVSASVGDRVTITCRASQAIGSWLAWYQQKPGKAPKLLISSASSLQSGVPSRFSGSGSGTDFTLTISSLQPEDFATYYCQQLDSYPVTFGPGTKVDIK

SEQ ID NO: 130 - protein, 125 aa, Artificial Sequence
  source: /note=“Description of Artificial Sequence Synthetic polypeptide”
EVQLLESGGGLIQPGGSLRLSCAASGLTVSSNYMSWVRQAPGKGLEWVSVIYSGGSTYYADSVKGRFTISRDNSKNTLYLQMNSLRAEDTAVYYCARAIDSGAFDIWGQGTMVTVSSGSASAPTL

SEQ ID NO: 131 - DNA, 735 bp, Artificial Sequence
  source: /note=“Description of Artificial Sequence Synthetic polynucleotide”
gagctcgtgttgacacagtctccaggcaccctgtctttgtctccaggggaaagagccaccctctcctgcagggccagtcagagtctttacaacaatttcttagcctggtaccagcagaaacctggccaggctcccaggctcctaatctatgctgcatccaacagggccactggcatcccagacaggttcagtggcagtgggtctgggacagacttcactctcaccatcagcagactggagcctgaagattttgcagtgtattactgtcagcagtatggtagctcacctgggtacacttttggccaggggaccaagctggagatcaaaggtggttcctctagatcttcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagaacccttagacaatactactactacggtatggacgtctggggccaagggaccacggtcaccgtctcctcagggagtgcatccgccccaaccctc

SEQ ID NO: 132 - DNA, 726 bp, Artificial Sequence
  source: /note=“Description of Artificial Sequence Synthetic polynucleotide”
gagctcatgctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggataacaccttattattcggcggaggcaccgagctgaccgtcctcggtggtggttcctctagatcttccggggtgcagctggtggagtctgggggaggcttggtccagcctggggggtccctgagactctcctgttcagcctctggatttaccttcagtagctatgccatgcactgggtccgccaggctccagggaagggactggaatacgtttcaggtattagtagtaatgggggtggcacacactatgcagactccgtgaagggcagattcagcatctccagagacaattccaagaacacgctgtatcttcaaatgagcagtctgagagctgaggacacggctgtgtattactgtgtgaaagatgatagtgggagctacttctccctatttgaccactggggccagggaaccctggtcaccgtctcctcagcctccaccaagggcccatcggtc

SEQ ID NO: 133 - DNA, 723 bp, Artificial Sequence
  source: /note=“Description of Artificial Sequence Synthetic polynucleotide”
gagctccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagcctgcagcctgaagattttgcaacttactattgtcaacaggctaacagtttccccttcactttcggccctgggaccaaagtggatatcaaaggtggttcctctagatcttcccaggtgcagctggtgcagtctggggctgaggtgaagaagcctggggcctcagtgaaggtctcctgcaaggcttctggttacacctttaccagctatggtatcagctgggtgcgacaggcccctggacaagggcttgagtggatgggatgggtgaaccccaatggtggtcacaccggctatgcacagaagttccagggcagggtcaccatgacccggcacaccgccaccaacacagtctacatggaactgagtagcctgacatctgacgacacggccgtctattactgtgcgcggggaaagggctactactacgggatggacgtctggggccaagggaccacggtcctcgtctcgtcagcttccaccaagggcccatcggtc

SEQ ID NO: 134 - DNA, 744 bp, Artificial Sequence
  source: /note=“Description of Artificial Sequence Synthetic polynucleotide”
gagctccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactattgtcaacaggctaacagtttccctctcactttcggcggagggaccaaggtggaaatcaaaggtggttcctctagatcttcccaggtgcagctggtgcagtctggggctgaggtgaagaagcctgggtcctcggtgaaggtctcctgcaaggcttctggaggcaccctcagtgaaaatggtttcagctgggtgcgacaggcccctggacaaggacttgagtggatgggccgaatcgtccctatgtttgacatatcaaactacgcagagaggttccaagacagagtcacgattaccgcggacgggtccacgaacacagcctacatggagctgcgcagtttgagattggaggacacggccgtatattactgtgcgagagacgcggtggctacttccccgggggtttatcagcactacggtatggacgtctggggccaagggaccacggtcaccgtctcctcagcctccaccaagggcccatcggtc

SEQ ID NO: 135 - DNA, 723 bp, Artificial Sequence
  source: /note=“Description of Artificial Sequence Synthetic polynucleotide”
gagctcgtgatgactcagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccccctcactttcggccctgggaccaaagtggatatcaaaggtggttcctctagatcttccgaggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagccaggggactggggtgggggagtgactactggggccagggaaccctggtcaccgtctcctcagggagtgcatccgccccaaccctc

SEQ ID NO: 136 - DNA, 717 bp, Artificial Sequence
  source: /note=“Description of Artificial Sequence Synthetic polynucleotide”
gagctcgtgatgactcagtctccatcttccgtgtctgcatctgtaggagacagagtcaccatcacttgtcgggcgagtcaggctattggcagctggttagcctggtatcagcagaaaccagggaaagcccccaagctcctcatctcttctgcatccagtttgcaaagtggggtcccatcaaggtttagcggcagtggatctgggacagatttcactctcaccatcagcagcctgcagcctgaagattttgcaacttattactgtcaacagcttgatagttaccctgtcactttcggccctgggaccaaagtggatatcaaaggtggttcctctagatcttccgaggtgcagctgttggagtctggaggaggcttgatccagcctggggggtccctgagactctcctgtgcagcctctgggttaaccgtcagtagcaactacatgagttgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgagagccattgactccggtgcttttgatatctggggccaagggacaatggtcaccgtctcttcagggagtgcatccgccccaaccctc

SEQ ID NO: 137 - protein, 11 aa, Homo sapiens
GNMWAQTWSNI

SEQ ID NO: 138 - protein, 11 aa, Homo sapiens
GDMWGRFWTNL

SEQ ID NO: 139 - protein, 11 aa, Homo sapiens
REVVCHASAWD

SEQ ID NO: 140 - protein, 11 aa, Homo sapiens
QKAVCHPTAWD

SEQ ID NO: 141 - protein, 11 aa, Equus przewalskii
GDMWGRFWTNL

SEQ ID NO: 142 - protein, 11 aa, Camelus ferus
GDMWGRFWTNL

SEQ ID NO: 143 - protein, 11 aa, Puma concolor
GDMWGRFWTNL

SEQ ID NO: 144 - protein, 11 aa, Ursus maritimus
GDMWGRFWTNL

SEQ ID NO: 145 - protein, 11 aa, Gorilla gorilla
GDMWGRFWTNL

SEQ ID NO: 146 - protein, 11 aa, Pan troglodytes
GDMWGRFWTNL

SEQ ID NO: 147 - protein, 11 aa, Macaca mulatta
GDMWGRFWTNL

SEQ ID NO: 148 - protein, 11 aa, Chinchilla lanigera
GDMWGRFWTNL

SEQ ID NO: 149 - protein, 11 aa, Marmota marmota
GDMWGRFWTNL

SEQ ID NO: 150 - protein, 11 aa, Oryctolagus cuniculus
GDMWGRFWTNL

SEQ ID NO: 151 - protein, 11 aa, Myotis lucifugus
GDMWGRFWTNL

SEQ ID NO: 152 - protein, 11 aa, Equus przewalskii
RKVVCHPTAWD

SEQ ID NO: 153 - protein, 11 aa, Camelus ferus
RKVVCHPTAWD

SEQ ID NO: 154 - protein, 11 aa, Puma concolor
QKVVCHPTAWD

SEQ ID NO: 155 - protein, 11 aa, Ursus maritimus
QKVVCHPTAWD

SEQ ID NO: 156 - protein, 11 aa, Gorilla gorilla
QKAVCHPTAWD

SEQ ID NO: 157 - protein, 11 aa, Pan troglodytes
QKAVCHPTAWD

SEQ ID NO: 158 - protein, 11 aa, Macaca mulatta
QKVVCHPTAWD

SEQ ID NO: 159 - protein, 11 aa, Chinchilla lanigera
RKVVCHPTAWD

SEQ ID NO: 160 - protein, 11 aa, Marmota marmota
RKVVCHPTAWD

SEQ ID NO: 161 - protein, 11 aa, Oryctolagus cuniculus
RKVVCHPTAWD

SEQ ID NO: 162 - protein, 11 aa, Myotis lucifugus
RKVVCHPTAWD